(19) United States
(12) Patent Application Publication
(10) Pub. No.: US 2026/0116880 A1
(43) Pub. Date: Apr. 30, 2026

(54) LIPIDATED TLR7/8 MODULATORS AS ADJUVANTS AND USES THEREOF

(71) Applicant: Pfizer Inc., New York, NY (US)

(72) Inventors: Derek William Bartlett, Chapel Hill, NC (US); Bo Chen, Ridgewood, NJ (US); Matthew Scott Dowling, Old Lyme, CT (US); David Christopher Ebner, Newton, MA (US); KariAnn Sweeney Efferen, Lagrangeville, NY (US); Mark Edward Flanagan, Gales Ferry, CT (US); David Hepworth, Concord, MA (US); Ksenia Krylova, New Windsor, NY (US); Seyedehmahsa Moghimi, Paterson, NJ (US); Shibu Simon Philip, New Milford, NJ (US); Shuai Shi, Whippany, NJ (US); Raphael Simon, Upper Saddle River, NJ (US); Yujie Su, Nanuet, NY (US); Joseph Scott Warmus, Ledyard, CT (US); Sabine Susanne Wellnitz, Nanuet, NY (US)

(21) Appl. No.: 19/366,670

(22) Filed: Oct. 23, 2025

Related U.S. Application Data
(60) Provisional application No. 63/711,458, filed on Oct. 24, 2024.
(60) Provisional application No. 63/886,202, filed on Sep. 23, 2025.

(51) Int. Cl. C07D 471/04; A61K 9/1277; A61K 31/4745; A61K 31/496; A61K 39/00; A61K 39/09; A61P 37/04

(52) U.S. Cl. CPC C07D 471/04; A61K 9/1277; A61K 31/4745; A61K 31/496; A61K 39/092; A61P 37/04; A61K 2039/55555; A61K 2039/6037

(57) ABSTRACT

The disclosure relates to compounds of Formula (I)

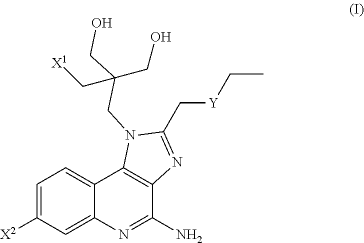

and pharmaceutically acceptable salts thereof, wherein X¹, X², and Y are as defined in the description; to their use in medicine; to compositions containing them; to processes for their preparation; and to intermediates used in such processes. The compounds of Formula (I) may modulate the activity of antigens of interest and may be useful in inducing or enhancing an immune response against diseases, disorders and conditions mediated by antigens of interest. In a particular embodiment, the compounds of Formula (I) may be useful as a component of a liposomal adjuvant formulation.

### RELATED APPLICATIONS

This application claims priority to U.S. Provisional Application No. 63/711,458 filed Oct. 24, 2024 and U.S. Provisional Application No. 63/886,202 filed Sep. 23, 2025. The entire content of each of the foregoing applications is herein incorporated by reference in its entirety.

### BACKGROUND

The present disclosure relates to novel lipidated Toll-likelin receptor 7 (TLR7), Toll-like receptor 8 (TLR8), and Toll-like receptor 7/8 (TLR7/8) modulating adjuvant compounds. The disclosure also relates to the preparation of the compounds and intermediates used in the preparation, compositions containing the compounds, and uses of the compounds, including as adjuvants for antigens of interest.

Studies and research regarding adjuvant use as a vaccine component have significantly increased nowadays. Adjuvants are compounds that enhance immune system activation and recognition of a vaccine's active component, concerning subunit-based vaccines, as well as RNA-based vaccines.

Adjuvants act by enhancing the innate immune system's response magnitude, breadth, and durability. The potency of adjuvants is closely related to their ability to be recognized as pathogens/foreign bodies through pattern recognition receptors (PRRs). It is expected that a vaccine's active component will become recognized, thereby triggering a specific and long-lasting immune response to be mounted through the adaptive immune system (Excler et al., Nat. Med., 27 (2021), pp. 591-600).

Toll-like receptors (TLRs) are a family of transmembrane proteins that recognize structurally conserved molecules that are derived from and unique to pathogens, referred to as pathogen-associated molecular patterns (PAMPs), a sub-class of PRR. As such, TLRs function in the mammalian immune system as front-line sensors of pathogen-associated molecular patterns, detecting the presence of invading pathogens (Takeuchi and Akira 2010 Cell 140:805-820). TLR engagement in sentinel immune cells causes biosynthesis of selected cytokines (e.g., type I interferons), induction of costimulatory molecules, and increased antigen presentation capacity. These are important molecular mechanisms that activate innate and adaptive immune responses. Accordingly, agonists and antagonists of TLRs find use in modulating immune responses. TLR agonists are typically employed to stimulate immune responses, whereas TLR antagonists are typically employed to inhibit immune responses (Gosu etal 2012. Molecules 17:13503-13529).

The human genome contains 10 known functional TLRs, of these TLR3, TLR7, TLR8, and TLR9 sense nucleic acids and their degradation products. The distribution of TLR7, TLR8, and TLR9 is restricted to the endosomal compartments of cells and they are preferentially expressed in cells of the immune system. In the activated, dimeric receptor configuration TLR7 and TLR8 recognize single strand RNA at one ligand binding site and the ribonucleoside degradation products guanosine and uridine, respectively, (as well as small molecule ligands with related structural motifs) at a second ligand binding site (Zhang et al 2016 Immunity 45 (4); 737-748: Tanji et al 2015 Nat Struct Mol Biol 22:109-115). Engagement of TLR7 in plasmacytoid dendritic cells leads to the induction of Type I interferon, which plays essential functions in the control of the adaptive immune response (Bao and Liu 2013 Protein Cell 4:40-5). Engagement of TLR8 in myeloid dendritic cells, monocytes and monocyte-derived dendritic cells induces a prominent pro-inflammatory cytokine profile, characterized by increased production of tumor necrosis factor alpha, interleukin-12, and IL-18 (Eigenbrod et al J Immunol, 2015, 195, 1092-1099). Thus, virtually all major types of monocytic and dendritic cells can be activated by agonists of TLR7 and TLR8 to become highly effective antigen-presenting cells, thereby promoting an effective innate and adaptive immune response. Most antigen presenting cell types express only one of these two receptors, accordingly small molecules with potent agonist activity against both TLR7 and TLR8 receptors are potentially more effective immune adjuvants than agonists specific for only one of these TLRs. Thus, a TLR7/TLR8 (TLR7/8) small molecule agonist with dual bioactivity would cause innate immune responses in a wider range of antigen presenting cells and other key immune cell types, including plasmacytoid and myeloid dendritic cells, monocytes, and B cells (van Haren et al 2016 J Immunol 197:4413-4424; Ganapathi et al 2015 Plos One 10 (8).e0134640).

Albumin has been extensively studied as a natural vector for lymph node targeted drug delivery (Adv. Drug Delivery Rev. 2018, 130, 73-89). First, albumin (about 7 nm in size) is the most abundant protein in the blood, reaching a concentration of 40 mg/mL, but maintaining a relatively lower concentration, about 14 mg/mL, in the interstitial fluid. This concentration difference tends to drive albumin to the lymphatics instead of blood capillaries. Second, albumin has an extraordinarily long half-life and is continuously synthesized in the liver for circulation. Third, natural ligands, such as long aliphatic fatty acids and hydrophobic molecules, have been discovered to bind albumin with their complexed crystal structures also being resolved. The albumin hitchhiking lymph node targeting was first demonstrated with TLR9 ligands conjugated to lipids and natural hydrophobic molecules such as cholesterol (Nature 507, 519-522, 2014).

What is needed in the field is a TLR7/8 agonist targeted to accumulate in the lymph node to (a) activate the immune cells in the lymph node to adjuvant the vaccine antigen, while (b) minimizing systemic exposure to the TLR7/8 agonist in order to circumvent systemic inflammation.

Accordingly, there remains a need for improved adjuvants targeted to accumulate in the lymph node. The current disclosure relates to the use of potent dual TLR7/8 agonists conjugated to a lipid.

### SUMMARY OF THE DISCLOSURE

The present disclosure provides, in part, compounds of Formula (I) and pharmaceutically acceptable salts thereof. Such compounds may agonize or modulate the activity of TLR7 and/or TLR8 and may be useful as vaccine adjuvants. Also provided are pharmaceutical compositions comprising the compounds or salts of the disclosure, alone or in combination with additional therapeutic agents. The present disclosure also provides, in part, methods for preparing such compounds, pharmaceutically acceptable salts and compositions of the disclosure, and methods of using the foregoing. This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

According to an embodiment of the disclosure there is provided a compound of Formula (I)

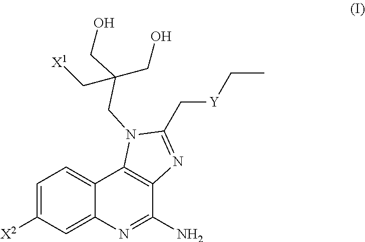

or a pharmaceutically acceptable salt thereof, wherein:
Y is —O— or —CH₂—; and
one of X¹ and X² is H, and the other of X¹ and X² has a formula selected from the group consisting of formula (a), formula (b), and formula (c):

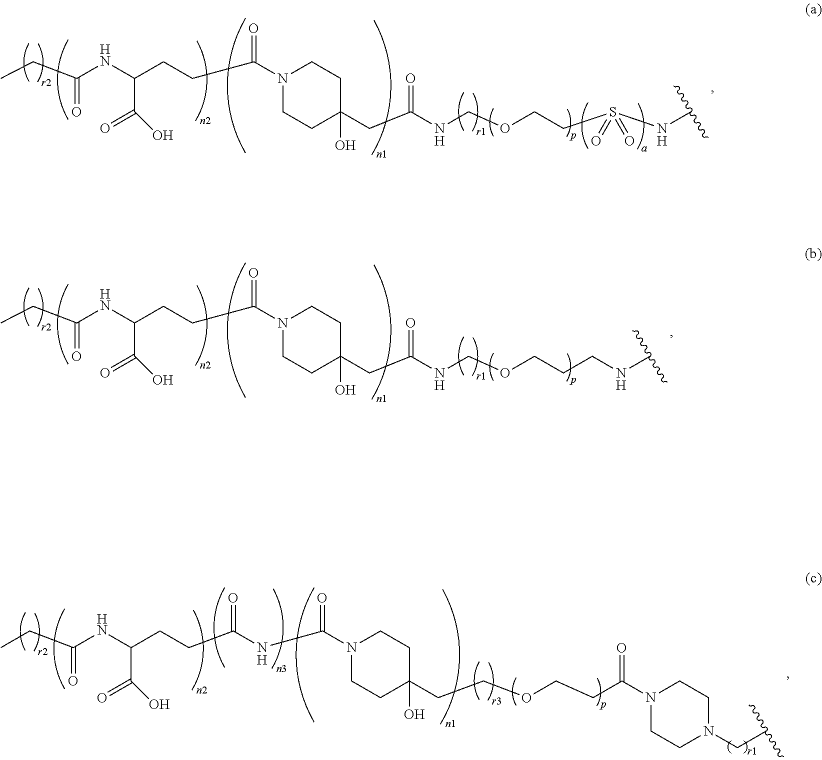

wherein:
a is 0 or 1;
r1 is an integer from 2 to 6;
r2 is an integer from 10 to 20;
r3 is an integer from 0 to 6;
n1 is 0 or 1 and n2 is 0 or 1, wherein at least one of n1 and n2 is 1;
n3 is 0 or 1; and
p is an integer from 0 to 6.

Described below are embodiments of the disclosure, where for convenience Embodiment 1 (E1) is identical to the embodiment of Formula (I) provided above.

It is to be understood that both the foregoing general description and the following detailed description are exemplary and explanatory only and are not restrictive of the disclosure, as claimed.

### DETAILED DESCRIPTION OF THE DISCLOSURE

The lipidated compounds, combinations, and methods of the present disclosure are believed to have one or more advantages, such as delivering the TLR7/8 modulating adjuvant to the lymph node via albumin trafficking. Without being bound to a particular mechanism or theory, the addition of a lipid moiety to the TLR7/8 modulating molecule may enhance binding of said molecule to interstitial albumin at the injection site. Binding to interstitial albumin may in turn, enhance trafficking of the TLR7/8 modulating molecule to the draining lymph node through afferent lymphatic vessels. Retention of the adjuvant in the lymph node via albumin trafficking may provide sustained exposure and accompanied stimulation of the immune system to allow for optimal immune response to the vaccine antigen.

Furthermore, the lipidated compounds of the present disclosure provide advantages versus non-lipidated compounds in facilitating the integration of said compounds into a liposomal adjuvant formulation. For example, when the compounds of the disclosure are combined with lipid components (i.e., phospholipids, cholesterols, etc.), the hydrophobic moieties within the TLR 7/8 modulating compounds of the present disclosure may facilitate the formation of a liposome comprising said compounds. Without being bound to a particular mechanism or theory, via integration into a liposome, the lipidated compounds of the present disclosure may have an enhanced ability to move throughout the lymphatic system which results in increased adjuvant activity.

The present disclosure may be understood more readily by reference to the following detailed description of the embodiments of the disclosure and the Examples included herein. It is to be understood that this disclosure is not limited to specific synthetic methods of making that may of course vary. It is to be also understood that the terminology used herein is for the purpose of describing specific embodiments only and is not intended to be limiting.

E1 A compound of Formula (I) or a pharmaceutically acceptable salt thereof, as defined above.

E2 The compound of embodiment E1, wherein X¹ has formula a.

E3 The compound of embodiment E2, wherein a is 1, n1 is 0, and n2 is 1.

E4 The compound of any one of embodiments E1-E3, wherein r1 is 4.

E5 The compound of any one of embodiments E1-E4, wherein p is 0.

E6 The compound of embodiment E2, wherein a is 1, n1 is 0, and n2 is 1.

E7 The compound of embodiment E6, wherein r1 is 2 and p is 3.

E8 The compound of embodiment E1, wherein X¹ has formula b.

E9 The compound of embodiment E8, wherein n1 is 0 and n2 is 1.

E10 The compound of embodiment E9, wherein r1 is 2 and p is 3.

E11 The compound of embodiment E8, wherein n1 is 1 and n2 is 1.

E12 The compound of embodiment E11, wherein r1 is 3 and p is 0.

E13 The compound of embodiment E1, wherein X² has formula c.

E14 The compound of embodiment E13, wherein n1 is 1, n2 is 0, and n3 is 0.

E15 The compound of embodiment E14, wherein r1 is 3, r3 is 0, and p is 0.

E16 The compound of embodiment E13, wherein n1 is 0, n2 is 1, and n3 is 1.

E17 The compound of embodiment E16, wherein r1 is 3, r3 is 2, and p is 3.

E18 The compound of any one of embodiments E1-E17, or a pharmaceutically acceptable salt thereof, wherein r2 is 13, 14, or 15.

E19 The compound of any one of embodiments E1-E18, or a pharmaceutically acceptable salt thereof, wherein r2 is 14.

E20 A compound, which is N5-(4-(N-(3-(4-Amino-2-butyl-1H-imidazo[4,5-c]quinolin-1-yl)-2,2-bis(hydroxymethyl)propyl)sulfamoyl)butyl)-N2-palmitoyl-L-glutamine, or a pharmaceutically acceptable salt thereof.

E21 A compound, which is(S)-1-(N-(3-(4-Amino-2-butyl-1H-imidazo[4,5-c]quinolin-1-yl)-2,2-bis(hydroxymethyl)propyl)sulfamoyl)-13-oxo-16-palmitamido-3,6,9-trioxa-12-azaheptadecan-17-oic acid, or a pharmaceutically acceptable salt thereof.

E22 A compound, which is(S)-1-(4-Amino-2-butyl-1H-imidazo[4,5-c]quinolin-1-yl)-2,2-bis(hydroxymethyl)-18-oxo-21-palmitamido-8, 11, 14-trioxa-4, 17-diazadocosan-22-oic acid, or a pharmaceutically acceptable salt thereof.

E23 A compound, which is(S)-5-(4-(2-((4-((3-(4-Amino-2-butyl-1H-imidazo[4,5-c]quinolin-1-yl)-2,2-bis(hydroxymethyl)propyl)amino)butyl) amino)-2-oxoethyl)-4-hydroxypiperidin-1-yl)-5-oxo-2-palmitamidopentanoic acid, or a pharmaceutically acceptable salt thereof.

E24 A compound, which is 1-(4-(2-(4-(3-(4-Amino-2-butyl-1-(3-hydroxy-2-(hydroxymethyl)-2-methylpropyl)-1H-imidazo[4,5-c]quinolin-7-yl)propyl) piperazin-1-yl)-2-oxoethyl)-4-hydroxypiperidin-1-yl) hexadecan-1-one, or a pharmaceutically acceptable salt thereof.

E25 A compound, which is(S)-1-(4-(3-(4-Amino-2-(ethoxymethyl)-1-(3-hydroxy-2-(hydroxymethyl)-2-methylpropyl)-1H-imidazo[4,5-c]quinolin-7-yl)propyl) piperazin-1-yl)-1, 14-dioxo-17-palmitamido-4,7,10-trioxa-13-azaoctadecan-18-oic acid, or a pharmaceutically acceptable salt thereof.

E26 A pharmaceutical composition comprising the compound according to any one of embodiments E1-E25, or a pharmaceutically acceptable salt thereof, and at least one pharmaceutically acceptable excipient.

E27 A crystalline form of the compound according to any one of embodiments E1-E25 or a pharmaceutically acceptable salt thereof.

E28 A method of inducing an immune response to an antigen of interest in a subject, comprising administering to the subject the pharmaceutical composition of embodiment E26, wherein the composition further comprises the antigen of interest.

E29 A method for immunizing a subject against a disease or disorder caused by or associated with an antigen of interest, comprising administering to the subject the pharmaceutical composition of embodiment E26, wherein the composition further comprises the antigen of interest.

E30 A method for preventing a disease or disorder caused by or associated with an antigen of interest in a subject, comprising administering to the subject the pharmaceutical composition of embodiment E26, wherein the composition further comprises the antigen of interest.

E31 A method for treating a disease or disorder caused by or associated with an antigen of interest in a subject, comprising administering to the subject the pharmaceutical composition of embodiment E26, wherein the composition further comprises the antigen of interest.

E32 A method for increasing an immune response to an antigen of interest in a subject, comprising administering to the subject the pharmaceutical composition of embodiment E26, wherein the composition further comprises the antigen of interest.

E33 The method of any one of embodiments E28 to E32, wherein the antigen of interest is an infectious disease antigen.

E34 The method of any one of embodiments E28 to E33, wherein the antigen of interest is a viral antigen, a bacterial antigen, a fungal antigen, or a parasitic antigen.

E35 The method of any one of embodiments E28 to E32, wherein the antigen of interest is a cancer antigen.

E36 The method of any one of embodiments E28-E35, wherein the method induces an immune response in the subject to the antigen of interest and the immune response is at least 10%, at least 20%, at least 30%, at least 40%, at least 50%, at least 60%, at least 70%, at least 80%, at least 90%, or at least 100%, higher than the immune response of the subject induced by a composition comprising the antigen of interest without the compound according to any one of embodiments E1 to E25.

E37 The method of embodiment E36, wherein the immune response is measured by opsonophagocytic activity (OPA) geometric mean antibody titers.

E38 The method of embodiment E36, wherein the immune response is measured by neutralization geometric mean antibody titers.

E39 The method of any one of embodiments E28 to E38, wherein the subject is human.

E40 A compound according to any one of embodiments E1 to E25 for use as a medicament.

E41 A compound according to any one of embodiments E1 to E25 for use in inducing an immune response to an antigen of interest in a subject.

E42 A compound according to any one of embodiments E1 to E25 for use in immunizing a subject against a disease or disorder caused by or associated with an antigen of interest in a subject.

E43 A compound according to any one of embodiments E1 to E25 for use in preventing a disease or disorder caused by or associated with an antigen of interest in a subject.

E44 A compound according to any one of embodiments E1 to E25 for use in treating a disease or disorder caused by or associated with an antigen of interest in a subject.

E45 A compound according to any one of embodiments E1 to E25 for use in increasing an immune response to an antigen of interest in a subject.

E46 The compound according to any one of embodiments E40-E45, wherein the subject is a human.

E47 Use of a compound according to any one of embodiments E1 to E25 for the manufacture of a medicament for inducing an immune response to an antigen of interest in a subject.

E48 Use of a compound according to any one of embodiments E1 to E25 for the manufacture of a medicament for immunizing a subject against a disease or disorder caused by or associated with an antigen of interest.

E49 Use of a compound according to any one of embodiments E1 to E25 for the manufacture of a medicament for use in preventing a disease or disorder caused by or associated with an antigen of interest in the subject.

E50 Use of a compound according to any one of embodiments E1 to E25 for the manufacture of a medicament for treating a disease or disorder caused by or associated with an antigen of interest in the subject.

E51 Use of a compound according to any one of embodiments E1 to E25 for the manufacture of a medicament for increasing an immune response to an antigen of interest in a subject.

E52 The use according to any one of embodiments E47 to E51, wherein the subject is a human.

E53 A liposomal formulation, comprising a plurality of liposomes, wherein the liposomes comprise a compound of Formula (I):

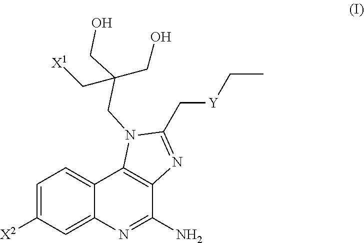

or a pharmaceutically acceptable salt thereof, wherein:
Y is —O— or —CH₂—; and
one of X¹ and X² is H, and the other of X¹ and X² has a formula selected from the group consisting of formula (a), formula (b), and formula (c):

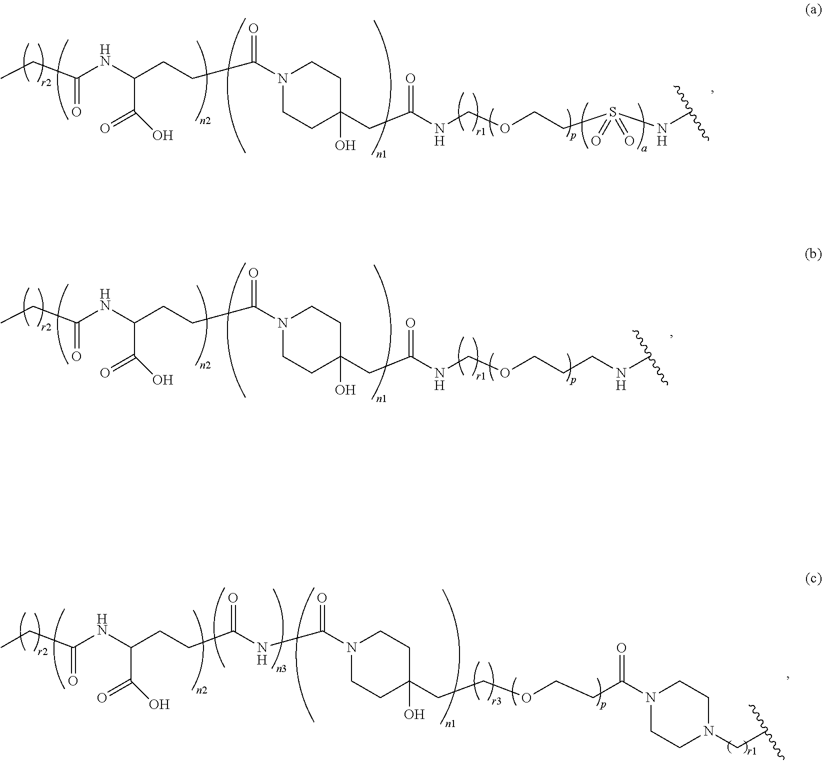

wherein:
a is 0 or 1;
r1 is an integer from 2 to 6;
r2 is an integer from 10 to 20;
r3 is an integer from 0 to 6;
n1 is 0 or 1 and n2 is 0 or 1, wherein at least one of n1 and n2 is 1;
n3 is 0 or 1; and
p is an integer from 0 to 6.

E54 The liposomal formulation of E53, wherein X¹ has formula a.

E55 The liposomal formulation of E54, wherein a is 1, n1 is 0, and n2 is 1.

E56 The liposomal formulation of any one of E53-E55, wherein r1 is 4.

E57 The liposomal formulation of any one of E53-E56, wherein p is 0.

E58 The liposomal formulation of E54, wherein a is 1, n1 is 0, and n2 is 1.

E59 The liposomal formulation of E58, wherein r1 is 2 and p is 3.

E60 The liposomal formulation of E53, wherein X¹ has formula b.

E61 The liposomal formulation of E60, wherein n1 is 0 and n2 is 1.

E62 The liposomal formulation of E61, wherein r1 is 2 and p is 3.

E63 The liposomal formulation of E60, wherein n1 is 1 and n2 is 1.

E64 The liposomal formulation of E63, wherein r1 is 3 and p is 0.

E65 The liposomal formulation of E53, wherein X² has formula c.

E66 The liposomal formulation of E65, wherein n1 is 1, n2 is 0, and n3 is 0.

E67 The liposomal formulation of E66, wherein r1 is 3, r3 is 0, and p is 0.

E68 The liposomal formulation of E65, wherein n1 is 0, n2 is 1, and n3 is 1.

E69 The liposomal formulation of E68, wherein r1 is 3, r3 is 2, and p is 3.

E70 The liposomal formulation of any one of E53-E69, wherein r2 is 13, 14, or 15.

E71 The liposomal formulation of any one of E53-E70, wherein r2 is 14.

E72 A liposomal formulation, comprising a plurality of liposomes, wherein the liposomes comprise a compound, which is N⁵-(4-(N-(3-(4-Amino-2-butyl-1H-imidazo[4,5-c]quinolin-1-yl)-2,2-bis(hydroxymethyl)propyl)sulfamoyl)butyl)-N²-palmitoyl-L-glutamine, or a pharmaceutically acceptable salt thereof.

E73 A liposomal formulation, comprising a plurality of liposomes, wherein the liposomes comprise a compound, which is(S)-1-(N-(3-(4-Amino-2-butyl-1H-imidazo[4,5-c]quinolin-1-yl)-2,2-bis(hydroxymethyl)propyl)sulfamoyl)-13-oxo-16-palmitamido-3,6,9-trioxa-12-azaheptadecan-17-oic acid, or a pharmaceutically acceptable salt thereof.

E74 A liposomal formulation, comprising a plurality of liposomes, wherein the liposomes comprise a compound, which is(S)-1-(4-Amino-2-butyl-1H-imidazo[4,5-c]quinolin-1-yl)-2,2-bis(hydroxymethyl)-18-oxo-21-palmitamido-8, 11, 14-trioxa-4, 17-diazadocosan-22-oic acid, or a pharmaceutically acceptable salt thereof.

E75 A liposomal formulation, comprising a plurality of liposomes, wherein the liposomes comprise a compound, which is(S)-5-(4-(2-((4-((3-(4-Amino-2-butyl-1H-imidazo[4,5-c]quinolin-1-yl)-2,2-bis(hydroxymethyl)propyl)amino)butyl) amino)-2-oxoethyl)-4-hydroxypiperidin-1-yl)-5-oxo-2-palmitamidopentanoic acid, or a pharmaceutically acceptable salt thereof.

E76 A liposomal formulation, comprising a plurality of liposomes, wherein the liposomes comprise a compound, which is 1-(4-(2-(4-(3-(4-Amino-2-butyl-1-(3-hydroxy-2-(hydroxymethyl)-2-methylpropyl)-1H-imidazo[4,5-c]quinolin-7-yl)propyl) piperazin-1-yl)-2-oxoethyl)-4-hydroxypiperidin-1-yl) hexadecan-1-one, or a pharmaceutically acceptable salt thereof.

E77 A liposomal formulation, comprising a plurality of liposomes, wherein the liposomes comprise a compound, which is(S)-1-(4-(3-(4-Amino-2-(ethoxymethyl)-1-(3-hydroxy-2-(hydroxymethyl)-2-methylpropyl)-1H-imidazo[4,5-c]quinolin-7-yl)propyl) piperazin-1-yl)-1, 14-dioxo-17-palmitamido-4,7,10-trioxa-13-azaoctadecan-18-oic acid, or a pharmaceutically acceptable salt thereof.

E78 The liposomal formulation of any one of E53-E77, wherein the liposomes further comprise:
a) a phospholipid; and
b) cholesterol.

E79 The liposomal formulation of E78, wherein the phospholipid is selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), and distearyl phosphatidylglycerol (DSPG).

E80 The liposomal formulation of E78 or E79, wherein the liposomes comprise two phospholipids selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), and distearyl phosphatidylglycerol (DSPG).

E81 The liposomal formulation of any one of E78-E80, wherein the liposomes comprise DMPC.

E82 The liposomal formulation of any one of E78-E81, wherein the liposomes comprise DMPG.

E83 The liposomal formulation of any one of E78-E82, wherein the liposomes comprise DMPC and DMPG.

E84 The liposomal formulation of any one of E81-E83, wherein the concentration of DMPC in the liposomal formulation is between about 1 mg/mL and about 100 mg/mL.

E85 The liposomal formulation of any one of E81-E84, wherein the concentration of DMPC in the liposomal formulation is about 2.8 mg/mL, about 14 mg/mL, about 28 mg/mL, or about 56 mg/mL.

E86 The liposomal formulation of any one of E82-E85, wherein the concentration of DMPG in the liposomal formulation is between about 1 mg/ml and about 10 mg/mL.

E87 The liposomal formulation of any one of E82-E86, wherein the concentration of DMPG in the liposomal formulation is about 0.32 mg/mL, about 1.6 mg/mL, about 3.2 mg/mL, or about 6.4 mg/mL.

E88 The liposomal formulation of any one of E78-E87, wherein the liposomes comprise cholesterol.

E89 The liposomal formulation of E88, wherein the concentration of the cholesterol is between about 1 mg/mL and about 100 mg/mL.

E90 The liposomal formulation of E88 or E89, wherein the concentration of the cholesterol is about 2.16 mg/mL, about 10.8 mg/mL, about 21.6 mg/mL, or about 43.2 mg/mL.

E91 The liposomal formulation of any one of E78-E90, wherein the concentration of the compound in the liposomal formulation is between about 0.001 mg/ml and about 0.1 mg/mL.

E92 The liposomal formulation of any one of E78-E91, wherein the concentration of the compound in the liposomal formulation is between about 0.003 mg/ml and about 0.07 mg/mL.

E93 The liposomal formulation of any one of E78-E92, wherein the concentration of the compound in the liposomal formulation is about 0.0688 mg/mL, about 0.0344, about 0.0172 mg/mL, or about 0.0034 mg/mL.

E94 The liposomal formulation of any one of E78-E93, wherein the liposomal formulation comprises DMPC, DMPG, and cholesterol, and wherein the concentration of the compound in the liposomal formulation is about 0.0034 mg/mL, the concentration of the DMPC is about 2.8 mg/mL, the concentration of the DMPG is about 0.32 mg/mL, and the concentration of the cholesterol is about 2.16 mg/mL.

E95 The liposomal formulation of any one of E78-E93, wherein the liposomal formulation comprises DMPC, DMPG, and cholesterol, and wherein the concentration of the compound in the liposomal formulation is about 0.0172 mg/mL, the concentration of the DMPC is about 14 mg/mL, the concentration of the DMPG is about 1.6 mg/mL, and the concentration of the cholesterol is about 10.8 mg/mL.

E96 The liposomal formulation of any one of E78-E93, wherein the liposomal formulation comprises DMPC, DMPG, and cholesterol, and wherein the concentration of the compound in the liposomal formulation is about 0.0344 mg/mL, the concentration of the DMPC is about 28 mg/mL, the concentration of the DMPG is about 3.2 mg/mL, and the concentration of the cholesterol is about 21.6 mg/mL.

E97 The liposomal formulation of any one of E78-E93, wherein the liposomal formulation comprises DMPC, DMPG, and cholesterol, and wherein the concentration of the compound in the liposomal formulation is about 0.0688 mg/mL, the concentration of the DMPC is about 56 mg/mL, the concentration of the DMPG is about 6.4 mg/mL, and the concentration of the cholesterol is about 43.2 mg/mL.

E98 The liposomal formulation of any one of E78-E97, wherein the liposomes further comprise a saponin.

E99 The liposomal formulation of E98, wherein the saponin is QS-21.

E100 The liposomal formulation of E99, wherein the concentration of the QS-21 in the liposomal formulation is between about 0.05 mg/mL and about 1 mg/mL.

E101 The liposomal formulation of E99 or E100, wherein the concentration of the QS-21 in the liposomal formulation is selected from the group consisting of about 0.1 mg/mL, about 0.2 mg/mL, about 0.3 mg/mL, and about 0.4 mg/mL.

E102 The liposomal formulation of any one of E78-E101, wherein the liposomes have a mean diameter size less than about 200 nanometers (nm).

E103 The liposomal formulation of any one of E78-E102, wherein the liposomes have a mean diameter size between about 100 nm and about 160 nm.

E104 The liposomal formulation of E102 or E103, wherein the average diameter size of the liposomes is measured at time zero (T0), wherein TO is the time when the formation of the liposomes in the liposomal formulation is completed.

E105 The liposomal formulation of E104, wherein the liposomes maintain a mean diameter size that deviates by no more than about 20% of the value of their average size at time zero (T0) for at least about 3 months after TO.

E106 The liposomal formulation of any one of E78-E105, wherein the liposomes have a polydispersity index (PDI) of less than about 0.5.

E107 The liposomal formulation of any one of E78-E106, wherein the liposomes have a PDI between about 0.1 and about 0.3.

E108 The liposomal formulation of E106 or E107, wherein the PDI of the liposomes is measured at time zero (T0), wherein TO is the time when the formation of the liposomes in the liposomal formulation is completed.

E109 The liposomal formulation of E108, wherein the liposomes maintain a PDI that deviates by no more than about 40% of the value of their PDI at time zero (T0) for at least about 3 months after TO.

E110 An immunogenic composition comprising the liposomal formulation of any one of E78-E109 and an immunogen.

E111 The immunogenic composition of E110, wherein the immunogen is derived from Streptococcus pneumoniae.

E112 The immunogenic composition of E111, wherein the immunogen is a Streptococcus pneumoniae serotype 3 polysaccharide.

E113 The immunogenic composition of E112, wherein the polysaccharide is conjugated to a C5a peptidase from Streptococcus (SCP) or a CRM197 carrier.

E114 The immunogenic composition of any one of E110-E113, wherein the composition comprises a phosphate and sodium chloride buffer.

E115 The immunogenic composition of E114, wherein the buffer comprises 10 mM phosphate and 150 mM sodium chloride and has a pH of about 6.2.

E116 A method of inducing an immune response in a subject against the immunogen, comprising administering to the subject the immunogenic composition of any one of E110-E115.

E117 A method for immunizing a subject against a disease or disorder caused by or associated with the immunogen, comprising administering to the subject the immunogenic composition of any one of E110-E115.

E118 A method for preventing a disease or disorder caused by or associated with the immunogen in a subject, comprising administering to the subject the immunogenic composition of any one of E110-E115.

E119 A method for treating a disease or disorder caused by or associated with the immunogen in a subject, comprising administering to the subject the immunogenic composition of any one of E110-E115.

E120 A method for increasing an immune response to the immunogen in a subject, comprising administering to the subject the immunogenic composition of any one of E110-E115.

E121 The method of any one of E116-E120, wherein the method induces an immune response in the subject to the immunogen and the immune response is at least about 10%, at least about 20%, at least about 30%, at least about 40%, at least about 50%, at least about 60%, at least about 70%, at least about 80%, at least about 90%, or at least about 100% higher than the immune response of the subject induced by a composition comprising the immunogen without the liposomal formulation of any one of E78-E109.

E122 The method of any one of E116-E120, wherein the method induces an immune response in the subject to the immunogen of interest and the immune response is at least about 150%, at least about 200%, at least about 250%, at least about 300%, at least about 350%, at least about 400%, at least about 450%, or at least about 500% higher than the immune response of the subject induced by a composition comprising the immunogen without the liposomal formulation of any one of E78-E109.

E123 The method of any one of E121 or E122, wherein the immune response is measured by opsonophagocytic activity (OPA) geometric mean antibody titers.

E124 The method of any one of E121 or E122, wherein the immune response is measured by neutralization geometric mean antibody titers.

E125 The method of any one of E116-E124, wherein the subject is human.

E126 The immunogenic composition of any one of E110-E115, for use as a medicament.

E127 The immunogenic composition of any one of E110-E115, for use in inducing an immune response to the immunogen in a subject.

E128 The immunogenic composition of any one of E110-E115, for use in immunizing a subject against a disease or disorder caused by or associated with the immunogen.

E129 The immunogenic composition of any one of E110-E115, for use in preventing a disease or disorder caused by or associated with the immunogen in a subject.

E130 The immunogenic composition of any one of E110-E115, for use in treating a disease or disorder caused by or associated with the immunogen in a subject.

E131 The immunogenic composition of any one of E110-E115, for use in increasing an immune response to the immunogen in a subject.

E132 Use of the immunogenic composition of any one of E110-E115, for the manufacture of a medicament for inducing an immune response to the immunogen in a subject.

E133 Use of the immunogenic composition of any one of E110-E115, for the manufacture of a medicament for immunizing a subject against a disease or disorder caused by or associated with the immunogen.

E134 Use of the immunogenic composition of any one of E110-E115, for the manufacture of a medicament for use in preventing a disease or disorder caused by or associated with the immunogen in a subject.

E135 Use of the immunogenic composition of any one of E110-E115, for the manufacture of a medicament for treating a disease or disorder caused by or associated with the immunogen in a subject.

E136 Use of the immunogenic composition of any one of E110-E115, for the manufacture of a medicament for increasing an immune response to the immunogen in a subject.

E137 A method for producing a liposomal formulation, comprising a plurality of liposomes, wherein the liposomes comprise (a) the compound of any one of E1-E25, (b) a saponin, (c) a phospholipid or phospholipids selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), and distearyl phosphatidylglycerol (DSPG), and (d) cholesterol, wherein the method comprises the following steps:
(i) dissolving the phospholipid(s), cholesterol, and the compound of any one of E1-E25 in an organic solvent to form an organic phase;
(ii) mixing the organic phase of step (i) into an aqueous phase, wherein the aqueous phase comprises a buffer or water, in a microfluidic device to form an intermediate liposome;
(iii) removing the organic solvent of the intermediate liposome of step (ii);
(iv) concentrating the intermediate liposome of step (iii);
(v) filtering the intermediate liposome of step (iv); and
(vi) aseptically mixing the intermediate liposome of step (v) and the saponin.

E138 The method of E137, wherein the phospholipid(s) of step (i) comprise DMPC and DMPG.

E139 The method of E137 or E138, wherein the aqueous phase of step (ii) comprises phosphate buffer and sodium chloride.

E140 The method of any one of E137-E139, wherein the microfluidic device of step (ii) comprises a Y-junction, T-junction, or coaxial mixer.

E141 The method of any one of E137-E140, wherein step (iii) and step (iv) comprise the use of tangential flow filtration (TFF).

E142 The method of any one of E137-E141, wherein step (v) comprises the use of bioburden reduction filtration (BBR), sterile filtration, or both.

E143 The method of any one of E137-E142, wherein the saponin of step (vi) is QS-21.

Furthermore, each of the embodiments described herein envisions within its scope pharmaceutically acceptable salts, tautomers, or stereoisomers thereof of the compounds described herein.

### Definitions

Unless otherwise defined herein, scientific and technical terms used in connection with the present disclosure have the meanings that are commonly understood by those of ordinary skill in the art.

The disclosure described herein suitably may be practiced in the absence of any element(s) not specifically disclosed herein.

“Compounds of the disclosure” include compounds of Formula (I) and the novel intermediates used in the preparation thereof. One of ordinary skill in the art will appreciate that compounds of the disclosure include conformational isomers (e.g., cis and trans isomers) and all optical isomers (e.g., enantiomers and diastereomers), racemic, diastereomeric and other mixtures of such isomers, tautomers thereof, where they may exist. One of ordinary skill in the art will also appreciate that compounds of the disclosure include solvates, hydrates, isomorphs, polymorphs, esters, salt forms, prodrugs, derivatives and isotopically labeled versions thereof, where they may be formed.

As used herein, the singular form “a”, “an”, and “the” include plural references unless indicated otherwise. For example, “a” substituent includes one or more substituents.

As used herein, the term “about” when used to modify a numerically defined parameter (e.g., the dose of 5 mg) means that the parameter may vary by as much as 10% below or above the stated numerical value for that parameter. For example, a dose of about 5 mg means 5 mg±10%, i.e., it may vary between 4.5 mg and 5.5 mg.

As used herein, the term “TLR 7/8 modulating” when used in reference to a compound indicates a compound that effects the activity of TLR 7 and TLR 8. For example, a compound that effects the activity of TLR 7 and TLR 8 can be an agonist or an antagonist. In some embodiments, a TLR 7/8 modulating compound disclosed herein is an agonist of TLR 7 and TLR 8.

If substituents are described as being “independently selected” from a group, each substituent is selected independent of the other. Each substituent therefore may be identical to or different from the other substituent(s).

“Optional” or “optionally” means that the subsequently described event or circumstance may, but need not occur, and the description includes instances where the event or circumstance occurs and instances in which it does not.

The terms “optionally substituted” and “substituted or unsubstituted” are used interchangeably to indicate that the particular group being described may have no non-hydrogen substituents (i.e., unsubstituted), or the group may have one or more non-hydrogen substituents (i.e., substituted). If not otherwise specified, the total number of substituents that may be present is equal to the number of H atoms present on the unsubstituted form of the group being described. Where an optional substituent is attached via a double bond, such as an oxo (═O) substituent, the group occupies two available valences, so the total number of other substituents that are included is reduced by two. In the case where optional substituents are selected independently from a list of alternatives, the selected groups may be the same or different. Throughout the disclosure, it will be understood that the number and nature of optional substituent groups will be limited to the extent that such substitutions make chemical sense to one of ordinary skill in the art.

“Halogen” or “halo” refers to fluoro, chloro, bromo and iodo (F, Cl, Br, I).

“Cyano” refers to a substituent having a carbon atom joined to a nitrogen atom by a triple bond, i.e., —C≡N.

“Hydroxy” refers to an —OH group.

“Oxo” refers to a double bonded oxygen (═O).

“Alkyl” refers to a saturated, monovalent aliphatic hydrocarbon radical that has a specified number of carbon atoms, including straight chain or branched chain groups. Alkyl groups may contain, but are not limited to, 1 to 12 carbon atoms (“C₁-C₁₂ alkyl”), 1 to 8 carbon atoms (“C₁-C₈alkyl”), 1 to 6 carbon atoms (“C₁-C₆ alkyl”), 1 to 5 carbon atoms (“C₁-C₅ alkyl”), 1 to 4 carbon atoms (“C₁-C₄ alkyl”), 1 to 3 carbon atoms (“C₁-C₃ alkyl”), or 1 to 2 carbon atoms (“C₁-C₂ alkyl”). Examples include, but are not limited to, methyl, ethyl, n-propyl, isopropyl, n-butyl, sec-butyl, isobutyl, tert-butyl, n-pentyl, isopentyl, neopentyl, n-hexyl, n-heptyl, n-octyl, and the like. Alkyl groups may be optionally substituted, unsubstituted or substituted, as further defined herein. In some instances, substituted alkyl groups are specifically named by reference to the substituent group. For example, “haloalkyl” refers to an alkyl group having the specified number of carbon atoms that is substituted by one or more halo substituents, up to the available valence number.

“Alkoxy” refers to an alkyl group, as defined herein, that is single bonded to an oxygen atom. The attachment point of an alkoxy radical to a molecule is through the oxygen atom. An alkoxy radical may be depicted as alkyl-O—. Alkoxy groups may contain, but are not limited to, 1 to 8 carbon atoms (“C₁-C₈ alkoxy”), 1 to 6 carbon atoms (“C₁-C₆ alkoxy”), 1 to 4 carbon atoms (“C₁-C₄ alkoxy”), or 1 to 3 carbon atoms (“C₁-C₃ alkoxy”). Alkoxy groups include, but are not limited to, methoxy, ethoxy, n-propoxy, isobutoxy, and the like.

“Alkenyl” refers to an alkyl group, as defined herein, consisting of at least two carbon atoms and at least one carbon-carbon double bond. For example, as used herein, the term “C₂-C₆ alkenyl” means straight or branched chain unsaturated radicals of 2 to 6 carbon atoms, including, but not limited to, ethenyl, 1-propenyl, 2-propenyl, 1-, 2-, or 3-butenyl, and the like. “Amino” refers to a group —NH₂, which is unsubstituted. Where the amino is described as substituted or optionally substituted, the term includes groups of the form-NRxRy, where each of Rx and Ry is defined as further described herein. For example, “alkylamino” refers to a group -NRxRy, wherein one of Rx and Ry is an alkyl moiety and the other is H, and “dialkylamino” refers to -NRxRy wherein both of Rx and Ry are alkyl moieties, where the alkyl moieties have the specified number of carbon atoms (e.g., —NH(C₁-C₄ alkyl) or —N(C₁-C₄ alkyl)₂).

“Aryl” or “aromatic” refers to monocyclic, bicyclic (e.g., biaryl, fused) or polycyclic ring systems that contain the specified number of ring atoms, in which all carbon atoms in the ring are of sp² hybridization and in which the pi electrons are in conjugation. Aryl groups may contain, but are not limited to, 6 to 20 carbon atoms (“C₆-C₂₀ aryl”), 6 to 14 carbon atoms (“C₆-C₁₄ aryl”), 6 to 12 carbon atoms (“C₆-C₁₂ aryl”), or 6 to 10 carbon atoms (“C₆-C₁₀ aryl”). Fused aryl groups may include an aryl ring (e.g., a phenyl ring) fused to another aryl ring. Examples include, but are not limited to, phenyl, biphenyl, naphthyl, anthracenyl, phenanthrenyl, indanyl, and indenyl. Aryl groups may be optionally substituted, unsubstituted or substituted, as further defined herein.

The term “pharmaceutically acceptable” means the substance (e.g., the compounds described herein) and any salt thereof, or composition containing the substance or salt of the disclosure is suitable for administration to a subject or patient.

The compounds of the disclosure have asymmetric carbon atoms. The carbon-carbon bonds of the compounds of the disclosure may be depicted herein using a solid line (—), a solid wedge ( [symbol] ) or a dotted wedge ( [symbol] ) The use of a solid line to depict bonds to asymmetric carbon atoms is meant to indicate that all possible stereoisomers (e.g., specific enantiomers, racemic mixtures, etc.) at that carbon atom are included. The use of either a solid or dotted wedge to depict bonds to asymmetric carbon atoms is meant to indicate that only the stereoisomer shown is meant to be included. It is possible that compounds of Formula (I) may contain more than one asymmetric carbon atom. In those compounds, the use of a solid line to depict bonds to asymmetric carbon atoms is meant to indicate that all possible stereoisomers are meant to be included. For example, unless stated otherwise, it is intended that the compounds of Formula (I) can exist as enantiomers and diastereomers or as racemates and mixtures thereof. The use of a solid line to depict bonds to one or more asymmetric carbon atoms in a compound of Formula (I) and the use of a solid or dotted wedge to depict bonds to other asymmetric carbon atoms in the same compound is meant to indicate that a mixture of diastereomers is present.

### Salts

Salts encompassed within the term “pharmaceutically acceptable salts” refer to the compounds of this disclosure which are generally prepared by reacting the free base or free acid with a suitable organic or inorganic acid, or a suitable organic or inorganic base, respectively, to provide a salt of the compound of the disclosure that is suitable for administration to a subject or patient.

In addition, the compounds of Formula I may also include other salts of such compounds which are not necessarily pharmaceutically acceptable salts, which may be useful as intermediates for one or more of the following: 1) preparing compounds of Formula I; 2) purifying compounds of Formula I; 3) separating enantiomers of compounds of Formula I; or 4) separating diastereomers of compounds of Formula I.

Suitable acid addition salts are formed from acids which form non-toxic salts. Examples include, but are not limited to, acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, 1,5-naphathalenedisulfonic acid and xinafoate salts.

Suitable base salts are formed from bases which form non-toxic salts. Examples include, but are not limited to aluminum, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulfate and hemicalcium salts.

For a review on suitable salts, see Paulekun, G. S. et al., Trends in Active Pharmaceutical Ingredient Salt Selection Based on Analysis of the Orange Book Database, J. Med. Chem. 2007; 50 (26), 6665-6672.

Pharmaceutically acceptable salts of compounds of the disclosure may be prepared by methods well known to one skilled in the art, including but not limited to the following procedures
(i) by reacting a compound of the disclosure with the desired acid or base;
(ii) by removing an acid- or base-labile protecting group from a suitable precursor of a compound of the disclosure or by ring-opening a suitable cyclic precursor, for example, a lactone or lactam, using the desired acid or base; or
(iii) by converting one salt of a compound of the disclosure to another. This may be accomplished by reaction with an appropriate acid or base or by means of a suitable ion exchange procedure.

These procedures are typically carried out in solution. The resulting salt may precipitate out and be collected by filtration or may be recovered by evaporation of the solvent.

### Solvates

The compounds of the disclosure, and pharmaceutically acceptable salts thereof, may exist in unsolvated and solvated forms. The term ‘solvate’ is used herein to describe a molecular complex comprising the compound of the disclosure, or a pharmaceutically acceptable salt thereof, and one or more pharmaceutically acceptable solvent molecules, for example, ethanol. The term ‘hydrate’ is employed when said solvent is water.

In addition, the compounds of Formula I may also include other solvates of such compounds which are not necessarily pharmaceutically acceptable solvates, which may be useful as intermediates for one or more of the following: 1) preparing compounds of Formula I; 2) purifying compounds of Formula I; 3) separating enantiomers of compounds of Formula I; or 4) separating diastereomers of compounds of Formula I.

A currently accepted classification system for organic hydrates is one that defines isolated site, channel, or metal-ion coordinated hydrates-see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex may have a well-defined stoichiometry independent of humidity. When, however, the solvent or water is weakly bound, as in channel solvates and hygroscopic compounds, the water/solvent content may be dependent on humidity and drying conditions. In such cases, non-stoichiometry will be the norm.

### Complexes

Also included within the scope of the disclosure are multi-component complexes (other than salts and solvates) wherein the drug and at least one other component are present in stoichiometric or non-stoichiometric amounts. In one embodiment, the compounds of the disclosure are in a complex with aluminum. In another embodiment, the compounds of the disclosure are in a complex with aluminum hydroxide. In another embodiment, the compounds of the disclosure are in a complex with aluminum phosphate. Complexes of this type include clathrates (drug-host inclusion complexes) and co-crystals. The latter are typically defined as crystalline complexes of neutral molecular constituents which are bound together through non-covalent interactions, for example, hydrogen bonded complex (cocrystal) may be formed with either a neutral molecule or with a salt. Co-crystals may be prepared by melt crystallization, by recrystallization from solvents, or by physically grinding the components together-see Chem Commun, 17; 1889-1896, by O. Almarsson and M. J. Zaworotko (2004). For a general review of multi-component complexes, see J Pharm Sci, 64 (8), 1269-1288, by Haleblian (August 1975).

### Solid Form

The compounds of the disclosure may exist in a continuum of solid states ranging from amorphous to crystalline. The term ‘amorphous’ refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

The compounds of the disclosure may also exist in a mesomorphic state (mesophase or liquid crystal) when subjected to suitable conditions. The mesomorphic state is intermediate between the true crystalline state and the true liquid state (either melt or solution) and consists of two dimensional order on the molecular level. Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4th Edition (Edward Arnold, 1970).

### Stereoisomers

Compounds of the disclosure may exist as two or more stereoisomers. Stereoisomers of the compounds may include cis and trans isomers (geometric isomers), optical isomers such as R and S enantiomers, diastereomers, rotational isomers, atropisomers, and conformational isomers. For example, compounds of the disclosure containing one or more asymmetric carbon atoms may exist as two or more stereoisomers. Where a compound of the disclosure contains an alkenyl or alkenylene group, geometric cis/trans (or Z/E) isomers are possible. Cis/trans isomers may also exist for saturated rings.

The pharmaceutically acceptable salts of compounds of the disclosure may also contain a counterion which is optically active (e.g., d-lactate or l-lysine) or racemic (e.g., dl-tartrate or dl-arginine).

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Alternatively, the racemate (or a racemic precursor) may be reacted with a suitable optically active compound, for example, an alcohol, or, in the case where a compound of the disclosure contains an acidic or basic moiety, a base or acid such as 1-phenylethylamine or tartaric acid. The resulting diastereomeric mixture may be separated by chromatography, fractional crystallization, or by using both of said techniques, and one or both of the diastereoisomers converted to the corresponding pure enantiomer(s) by means well known to a skilled person. Chiral compounds of the disclosure (and chiral precursors thereof) may be obtained in enantiomerically-enriched form using chromatography, typically HPLC Concentration of the eluate affords the enriched mixture. Chiral chromatography using sub- and supercritical fluids may be employed. Methods for chiral chromatography useful in some embodiments of the present disclosure are known in the art (see, for example, Smith, Roger M., Loughborough University, Loughborough, UK; Chromatographic Science Series (1998), 75 (Supercritical Fluid Chromatography with Packed Columns), pp. 223-249 and references cited therein).

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two crystal forms are produced in equimolar amounts each comprising a single enantiomer. While both of the crystal forms present in a racemic mixture have identical physical properties, they may have different physical properties compared to the true racemate. Racemic mixtures may be separated by conventional techniques known to those skilled in the art-see, for example, Stereochemistry of Organic Compounds by E. L. Eliel and S. H. Wilen (Wiley, 1994).

### Tautomerism

Where structural isomers are interconvertible via a low energy barrier, tautomeric isomerism (‘tautomerism’) may occur. This may take the form of proton tautomerism in compounds of the disclosure containing, for example, an imino/amino, keto/enol, or oxime/nitroso group, lactam/lactim or so-called valence tautomerism in compounds which contain an aromatic moiety. It follows that a single compound may exhibit more than one type of isomerism.

It must be emphasized that while, for conciseness, the compounds of the disclosure have been drawn herein in a single tautomeric form, all possible tautomeric forms are included within the scope of the disclosure.

### Isotopes

The present disclosure includes all pharmaceutically acceptable isotopically-labeled compounds of the disclosure wherein one or more atoms are replaced by atoms having the same atomic number, but an atomic mass or mass number different from the atomic mass or mass number which predominates in nature.

Examples of isotopes suitable for inclusion in the compounds of the disclosure may include isotopes of hydrogen, such as ²H (D, deuterium) and ³H (T, tritium), carbon, such as ¹¹C, ¹³C and ¹⁴C, chlorine, such as ³⁶Cl, fluorine, such as ₁₈F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S.

Certain isotopically-labelled compounds of the disclosure, for example those incorporating a radioactive isotope, are useful in one or both of drug or substrate tissue distribution studies. The radioactive isotopes, such as, tritium and ¹⁴C are particularly useful for this purpose in view of their ease of incorporation and ready means of detection. Substitution with positron emitting isotopes, such as, ¹¹C, ¹⁸F, ¹⁵O and ¹³N, may be useful in Positron Emission Topography (PET) studies for examining substrate receptor occupancy. Substitution with deuterium may afford certain therapeutic advantages resulting from greater metabolic stability, for example increased in vivo half-life, reduced dosage requirements, reduced CYP450 inhibition (competitive or time dependent), or an improvement in therapeutic index or tolerability.

In some embodiments, the disclosure provides deuterium-labeled (or deuterated) compounds and salts, where the formula and variables of such compounds and salts are each and independently as described herein. “Deuterated” means that at least one of the atoms in the compound is deuterium in an abundance that is greater than the natural abundance of deuterium (typically approximately 0.015%). A skilled artisan recognized that in chemical compounds with a hydrogen atom, the hydrogen atom actually represents a mixture of H and D, with about 0.015% being D. The concentration of the deuterium incorporated into the deuterium-labeled compounds and salt of the disclosure may be defined by the deuterium enrichment factor. It is understood that one or more deuterium may exchange with hydrogen under physiological conditions.

In some embodiments, one or more hydrogen atoms on certain metabolic sites on the compounds of the disclosure are deuterated.

Isotopically-labeled compounds of the disclosure may generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the disclosure include those wherein the solvent of crystallization may be isotopically substituted, e.g., D₂O, d₆-acetone, d₆-DMSO.

### Metabolites

Also included within the scope of the disclosure are active metabolites of compounds of the disclosure, that is, compounds formed in vivo upon administration of the drug, often by oxidation or dealkylation. Some examples of metabolites in accordance with the disclosure include, but are not limited to,
(i) where the compound of the disclosure contains an alkyl group, a hydroxyalkyl derivative thereof (—CH→—COH):
(ii) where the compound of the disclosure contains an alkoxy group, a hydroxy derivative thereof (—OR→—OH);
(iii) where the compound of the disclosure contains a tertiary amino group, a secondary amino derivative thereof (—NRR′→—NHR or —NHR′);
(iv) where the compound of the disclosure contains a secondary amino group, a primary derivative thereof (—NHR→—NH₂);
(v) where the compound of the disclosure contains a phenyl moiety, a phenol derivative thereof (—Ph→—PhOH);
(vi) where the compound of the disclosure contains an amide group, a carboxylic acid derivative thereof (—CONH₂→COOH); and
(vii) where the compound contains a hydroxy or carboxylic acid group, the compound may be metabolized by conjugation, for example with glucuronic acid to form a glucuronide. Other routes of conjugative metabolism exist. These pathways are frequently known as Phase 2 metabolism and include, for example, sulfation or acetylation. Other functional groups, such as NH groups, may also be subject to conjugation.

### Adjuvants

The compounds of the present disclosure can be used as adjuvants, for example within an immunogenic composition (i.e., vaccine). An adjuvant is a substance that enhances the immune response when administered together with an immunogen or antigen. Adjuvants may act primarily as a delivery system, primarily as an immune modulator or have strong features of both. Suitable adjuvants include those suitable for use in mammals, including humans. For example, adjuvants augment the intrinsic immune response to an immunogen without causing conformational changes in the immunogen that may affect the qualitative form of the immune response. In some embodiments, the adjuvant is a TLR 7/8 modulating molecule described herein.

An effective amount of an adjuvant, such as those described herein, refers to the amount necessary or sufficient to realize a desired biologic effect. For example, an effective amount of an adjuvant administered with an antigen for inducing an antigen-specific immune response is that amount necessary to induce an immune response in response to an antigen upon exposure to the antigen. Combined with the teachings provided herein, by choosing among the various adjuvants and weighing factors such as potency, relative bioavailability, subject body weight, severity of adverse side-effects and preferred mode of administration, an effective prophylactic or therapeutic treatment regimen can be planned which does not cause substantial toxicity and yet is effective to treat the particular subject. The effective amount for any particular application can vary depending on such factors as the disease or condition being treated, the particular adjuvant being administered, the size of the subject, or the severity of the disease or condition.

In some embodiments, a compound of the present disclosure can be used as an adjuvant in combination with one or more additional adjuvants. For example, in some embodiments, a compound of the present disclosure can be used as an adjuvant in combination with 1, 2, 3, 4, or more additional adjuvants. In some embodiments, a compound of the present disclosure can be used as an adjuvant in combination with an additional TLR modulating compound. For example, a compound of the present disclosure can be used as an adjuvant in combination with an additional TLR 7/8 modulating compound. In other embodiments, a compound of the present disclosure can be used as an adjuvant in combination with a TLR 4 modulating compound. In other embodiments, a compound of the present disclosure can be used as an adjuvant in combination with a TLR 5 modulating compound. In other embodiments, a compound of the present disclosure can be used as an adjuvant in combination with a TLR 9 modulating compound. In still other embodiments, a compound of the present disclosure can be used as an adjuvant in combination with a cytokine. In some embodiments, a compound of the present disclosure can be used as an adjuvant in combination with a saponin. In some embodiments, a compound of the present disclosure can be used as an adjuvant in combination with a compound that modulates the stimulator of interferon genes (STING) pathway. In other embodiments, a compound of the present disclosure can be used as an adjuvant in combination with a retinoic acid-inducible gene I (RIG-1) modulating compound. In yet other embodiments, a compound of the present disclosure can be used as an adjuvant in combination with aluminum. In one embodiment, a compound of the present disclosure can be used as an adjuvant in combination with aluminum phosphate. In another embodiment, a compound of the present disclosure can be used as an adjuvant in combination with aluminum hydroxide.

### Liposomal Adjuvants

In some embodiments, a TLR 7/8 modulating molecule described herein is combined with other components to form a liposomal formulation. As used herein, a liposomal formulation comprises liposomes. “Liposomes” as used herein refer to closed bilayer membranes containing an entrapped aqueous volume. Liposomes may also be uni-lamellar vesicles possessing a single membrane bilayer or multi-lamellar vesicles with multiple membrane bilayers, each separated from the next by an aqueous layer. The structure of the resulting membrane bilayer is such that the hydrophobic (non-polar) tails of the lipid are oriented toward the center of the bilayer while the hydrophilic (polar) heads orient towards the aqueous phase. Suitable hydrophilic polymers for surrounding the liposomes include, without limitation, PEG, polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxethylacrylate, hydroxymethylcellulose, hydroxyethylcellulose, polyethyleneglycol, polyaspartamide and hydrophilic peptide sequences as described in U.S. Pat. Nos. 6,316,024; 6, 126,966; 6,056,973; and 6,043,094. Liposomes can be made without hydrophilic polymers. Therefore, liposomal formulations may or may not contain hydrophilic polymers. Liposomes may be comprised of any lipid or lipid combination known in the art. For example, the vesicle-forming lipids may be naturally-occurring or synthetic lipids, including phospholipids, such as phosphatidylcholine, phosphatidylethanolamine, phosphatidic acid, phosphatidylserine, phosphatidylglycerol, phosphatidylinositol, and sphingomyelin as disclosed in U.S. Pat. Nos. 6,056,973 and 5,874,104.

When a liposomal adjuvant is used in a vaccine formulation, water-soluble antigens, such as proteins, peptides, nucleic acids, or carbohydrates, are encapsulated in the internal aqueous volume of the liposomes (See Tretiakova et al. Liposomes as Adjuvants and Vaccine Delivery Systems. Biochem (Mosc) Suppl Ser A Membr Cell Biol. 2022; 16 (1): 1-20). Alternatively, when a liposomal adjuvant is combined with lipophilic/amphiphilic substances, such as lipopeptides and glycolipids, these agents are embedded in the lipid bilayer (Id.) Depending on the type of molecule that is combined with the liposomal adjuvant, additional interactions can include associating with the surface of liposomes by adsorption or covalent binding (Id.) Accordingly, in some embodiments, a liposomal adjuvant comprises water-soluble antigens and the antigens are encapsulated in the internal aqueous volume of the liposomes. In some embodiments, water-soluble antigens are proteins, peptides, nucleic acids, or carbohydrates. In some embodiments, a liposomal adjuvant is combined with lipophilic or amphiphilic molecules and these molecules are embedded in the lipid bilayer. In some embodiments, the lipophilic or amphiphilic molecules embedded in the lipid bilayer of the liposome comprise cholesterol, fatty acids, or lipids. In some embodiments, the lipophilic or amphiphilic molecules embedded in the lipid bilayer are lipidated. In a particular embodiment, lipidated TLR 7/8 modulating molecules disclosed herein are embedded in the lipid bilayer of liposomes within a liposomal formulation to form a liposomal adjuvant.

In some embodiments, a TLR 7/8 modulating molecule described herein is combined with other components to form a liposomal adjuvant formulation. In some embodiments, a TLR 7/8 modulating molecule described herein is combined with a saponin to form a liposomal adjuvant formulation. In one embodiment, a TLR 7/8 modulating molecule described herein is combined with QS-21 to form a liposomal adjuvant formulation. In some embodiments, a TLR 7/8 modulating molecule described herein is combined with a phospholipid to form a liposomal adjuvant formulation. In some embodiments, a TLR 7/8 modulating molecule described herein is combined with at least one phospholipid selected from the group consisting of phosphatidylcholine (PC) and phosphatidylglycerol (PG) to form a liposomal adjuvant formulation. In some embodiments, a TLR 7/8 modulating molecule described herein is combined with at least one phospholipid selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), and distearyl phosphatidylglycerol (DSPG) to form a liposomal adjuvant formulation. In one embodiment, a TLR 7/8 modulating molecule described herein is combined with a sterol to form a liposomal adjuvant formulation. In one embodiment, a TLR 7/8 modulating molecule described herein is combined with cholesterol to form a liposomal adjuvant formulation. In some embodiments, a TLR 7/8 modulating molecule described herein is combined with a phospholipid, a saponin, and cholesterol to form a liposomal adjuvant formulation. In a particular embodiment, a TLR 7/8 modulating molecule described herein is combined with QS-21, cholesterol, and a phospholipid selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), and distearyl phosphatidylglycerol (DSPG) to form a liposomal adjuvant formulation. In another particular embodiment, a TLR 7/8 modulating molecule described herein is combined with QS-21, cholesterol, DMPC, and DMPG to form a liposomal adjuvant formulation.

In some embodiments, the liposomal formulations disclosed herein comprise a saponin. For the present embodiments, a suitable saponin is Quil A, its derivatives thereof, or any purified component thereof (for example, QS-7, QS-18, QS-21, or a mixture thereof). Quil A is a saponin preparation isolated from the South American tree Quillaja Saponaria Molina and was first found to have adjuvant activity. (See Dalsgaard et al., 1974, Archiv. für die gesanite Virusforschung, 44:243-254). Purified fragments of Quil A have been isolated by HPLC (See EP U.S. Pat. No. 362,279), including, for example, QS-7 and QS-21 (also known as QA7 and QA21, respectively). QS-21 is the 21st fraction purified from the sap of Quillaja Saponaria tree (See Qi et al. A Two-Step Orthogonal Chromatographic Process for Purifying the Molecular Adjuvant QS-21 with High Purity and Yield. J Chromatogr A. 2021 Jan. 4; 1635:461705). QS-21 has been shown to induce CD8+ cytotoxic T cells (CTLs), Th1 cells, and a predominant lgG2a antibody response (See Wong et al.; TCR Vaccines Against T Cell Lymphoma: QS-21 and IL-12 Adjuvants Induce a Protective CD8+ T Cell Response1. J Immunol 15 Feb. 1999; 162 (4): 2251-2258). In a preferred embodiment, a liposomal formulation comprises a TLR 7/8 modulating molecule described herein and QS-21.

In some embodiments, a liposomal formulation comprises a TLR 7/8 modulating molecule described herein and a saponin, wherein the concentration of the saponin is between about 0.01 mg/mL and about 10 mg/mL. In some embodiments, a liposomal formulation comprises a TLR 7/8 modulating molecule described herein and QS-21, wherein the concentration of the QS-21 is between about 0.01 mg/mL and about 10 mg/mL. In some embodiments, a liposomal formulation comprises a TLR 7/8 modulating molecule described herein and a saponin, wherein the concentration of the saponin is between about 0.05 m/mL and about 1 mg/mL. In some embodiments, a liposomal formulation comprises a TLR 7/8 modulating molecule described herein and QS-21, wherein the concentration of the QS-21 is between about 0.05 mg/mL and about 1 mg/mL. In some embodiments, a liposomal formulation comprises a TLR 7/8 modulating molecule described herein and a saponin, wherein the concentration of the saponin is between about 0.05 mg/mL and about 0.5 mg/mL. In some embodiments, a liposomal formulation comprises a TLR 7/8 modulating molecule described herein and QS-21, wherein the concentration of the QS-21 is between about 0.05 mg/mL and about 0.5 mg/mL. In some embodiments, a liposomal formulation comprises a TLR 7/8 modulating molecule described herein and a saponin, wherein the concentration of the saponin is about 0.05 mg/mL, about 0.1 mg/mL, about 0.15 mg/mL, about 0.2 mg/mL, about 0.25 mg/mL, about 0.3 mg/mL, about 0.35 mg/mL, about 0.4 mg/mL, about 0.45 mg/mL, or about 0.5 mg/mL. In some embodiments, a liposomal formulation comprises a TLR 7/8 modulating molecule described herein and QS-21, wherein the concentration of the QS-21 is about 0.05 mg/mL, about 0.1 mg/mL, about 0.15 mg/mL, about 0.2 mg/mL, about 0.25 mg/mL, about 0.3 mg/mL, about 0.35 mg/mL, about 0.4 mg/mL, about 0.45 mg/mL, or about 0.5 mg/mL. In a particular embodiment, a liposomal formulation comprises a TLR 7/8 modulating molecule described herein and a saponin, wherein the concentration of the saponin is about 0.1 mg/mL, about 0.2 mg/mL, about 0.3 mg/mL, or about 0.4 mg/mL. In another particular embodiment, a liposomal formulation comprises a TLR 7/8 modulating molecule described herein and QS-21, wherein the concentration of the QS-21 is about 0.1 mg/mL, about 0.2 mg/mL, about 0.3 mg/mL, or about 0.4 mg/mL. In another particular embodiment, a liposomal formulation comprises a TLR 7/8 modulating molecule described herein, QS-21, DMPC, DMPG, and cholesterol, wherein the concentration of the QS-21 is about 0.1 mg/mL, about 0.2 mg/mL, about 0.3 mg/mL, or about 0.4 mg/mL.

In some embodiments, a liposomal formulation comprises a TLR 7/8 modulating molecule described herein, wherein the concentration of the TLR 7/8 modulating molecule is between about 0.001 mg/mL and about 1 mg/mL. In some embodiments, a liposomal formulation comprises a TLR 7/8 modulating molecule described herein, wherein the concentration of the TLR 7/8 modulating molecule is between about 0.001 mg/ml and about 0.5 mg/mL. In some embodiments, a liposomal formulation comprises a TLR 7/8 modulating molecule described herein, wherein the concentration of the TLR 7/8 modulating molecule is between about 0.001 mg/mL and about 0.1 mg/mL. In some embodiments, a liposomal formulation comprises a TLR 7/8 modulating molecule described herein, wherein the concentration of the TLR 7/8 modulating molecule is between about 0.003 mg/mL and about 0.07 mg/mL. In some embodiments, a liposomal formulation comprises a TLR 7/8 modulating molecule described herein, wherein the concentration of the TLR 7/8 modulating molecule is about 0.001 mg/mL, about 0.002 mg/mL, about 0.003 mg/mL, about 0.004 mg/mL, about 0.005 mg/mL, about 0.006 mg/mL, about 0.007 mg/mL, about 0.008 mg/mL, about 0.009 mg/mL, about 0.01 mg/mL, about 0.02 mg/mL, about 0.03 mg/mL, about 0.04 mg/mL, about 0.05 mg/mL, about 0.06 mg/mL, about 0.07 mg/mL, about 0.08 mg/mL, about 0.09 mg/mL, or about 0.1 mg/mL.

In a particular embodiment, a liposomal formulation comprises a TLR 7/8 modulating molecule described herein, wherein the concentration of the TLR 7/8 modulating molecule is between about 0.06 mg/mL and about 0.07 mg/mL. In a particular embodiment, a liposomal formulation comprises a TLR 7/8 modulating molecule described herein, wherein the concentration of the TLR 7/8 modulating molecule is between about 0.068 mg/mL and about 0.069 mg/mL. In a particular embodiment, a liposomal formulation comprises a TLR 7/8 modulating molecule described herein, wherein the concentration of the TLR 7/8 modulating molecule is about 0.0688 mg/mL. In another particular embodiment, a liposomal formulation comprises a TLR 7/8 modulating molecule described herein, QS-21, DMPC, DMPG, and cholesterol, wherein the concentration of the TLR 7/8 modulating molecule is between about 0.06 mg/mL and about 0.07 mg/mL.

In a particular embodiment, a liposomal formulation comprises a TLR 7/8 modulating molecule described herein, wherein the concentration of the TLR 7/8 modulating molecule is between about 0.03 mg/mL and about 0.04 mg/mL. In a particular embodiment, a liposomal formulation comprises a TLR 7/8 modulating molecule described herein, wherein the concentration of the TLR 7/8 modulating molecule is between about 0.034 mg/mL and about 0.035 mg/mL. In a particular embodiment, a liposomal formulation comprises a TLR 7/8 modulating molecule described herein, wherein the concentration of the TLR 7/8 modulating molecule is about 0.0344. In another particular embodiment, a liposomal formulation comprises a TLR 7/8 modulating molecule described herein, QS-21, DMPC, DMPG, and cholesterol, wherein the concentration of the TLR 7/8 modulating molecule is between about 0.034 mg/ml and about 0.035 mg/mL.

In a particular embodiment, a liposomal formulation comprises a TLR 7/8 modulating molecule described herein, wherein the concentration of the TLR 7/8 modulating molecule is between about 0.01 mg/mL and about 0.02 mg/mL. In a particular embodiment, a liposomal formulation comprises a TLR 7/8 modulating molecule described herein, wherein the concentration of the TLR 7/8 modulating molecule is between about 0.017 mg/mL and about 0.018 mg/mL. In a particular embodiment, a liposomal formulation comprises a TLR 7/8 modulating molecule described herein, wherein the concentration of the TLR 7/8 modulating molecule is about 0.0172 mg/mL. In another particular embodiment, a liposomal formulation comprises a TLR 7/8 modulating molecule described herein, QS-21, DMPC, DMPG, and cholesterol, wherein the concentration of the TLR 7/8 modulating molecule is between about 0.017 mg/mL and about 0.018 mg/mL.

In a particular embodiment, a liposomal formulation comprises a TLR 7/8 modulating molecule described herein, wherein the concentration of the TLR 7/8 modulating molecule is between about 0.001 mg/mL and about 0.01 mg/mL. In a particular embodiment, a liposomal formulation comprises a TLR 7/8 modulating molecule described herein, wherein the concentration of the TLR 7/8 modulating molecule is between about 0.003 mg/mL and about 0.004 mg/mL. In a particular embodiment, a liposomal formulation comprises a TLR 7/8 modulating molecule described herein, wherein the concentration of the TLR 7/8 modulating molecule is about 0.0034 mg/mL. In another particular embodiment, a liposomal formulation comprises a TLR 7/8 modulating molecule described herein, QS-21, DMPC, DMPG, and cholesterol, wherein the concentration of the TLR 7/8 modulating molecule is between about 0.003 mg/ml and about 0.004 mg/mL.

In some embodiments, a liposomal formulation comprises a TLR 7/8 modulating molecule described herein and a lipid. In some embodiments, a liposomal formulation comprises a TLR 7/8 modulating molecule described herein and a lipid, wherein the lipid has only hydrophobic properties, for example, a triglyceride or a prenol lipid (i.e., squalene). In some embodiments, a liposomal formulation comprises a TLR 7/8 modulating molecule described herein and a lipid, wherein the lipid has amphipathic properties, for example, a phospholipid, cholesterol, a glycolipid, or a fatty acid.

In some embodiments, a liposomal formulation comprises a TLR 7/8 modulating molecule described herein and a phospholipid. In some embodiments, a liposomal formulation comprises a TLR 7/8 modulating molecule described herein and two phospholipids. In some embodiments, a liposomal formulation comprises a TLR 7/8 modulating molecule described herein and a phosphatidylcholine phospholipid (PC). In some embodiments, a liposomal formulation comprises a TLR 7/8 modulating molecule described herein and a phosphatidylethanolamine phospholipid (PE). In some embodiments, a liposomal formulation comprises a TLR 7/8 modulating molecule described herein and a phosphatidylserine phospholipid (PS). In some embodiments, a liposomal formulation comprises a TLR 7/8 modulating molecule described herein and a phosphatidylglycerol phospholipid (PG). In some embodiments, a liposomal formulation comprises a TLR 7/8 modulating molecule described herein and a sphingomyelin phospholipid. In some embodiments, a liposomal formulation comprises a TLR 7/8 modulating molecule described herein and a PC phospholipid, wherein the PC phospholipid is selected from the group consisting of dimyristoyl phosphatidylcholine (DMPC), dipalmitoyl phosphatidylcholine (DPPC), distearyl phosphatidylcholine (DSPC), and dioleoyl phosphatidylcholine (DOPC). In some embodiments, a liposomal formulation comprises a TLR 7/8 modulating molecule described herein and a PG phospholipid, wherein the PG phospholipid is selected from the group consisting of dimyristoyl phosphatidylglycerol (DMPG), dipalmitoyl phosphatidylglycerol (DPPG), and distearyl phosphatidylglycerol (DSPG). In some embodiments, a liposomal formulation comprises (i) TLR 7/8 modulating molecule described herein, (ii) a phosphatidylcholine phospholipid (PC) selected from the group consisting of dimyristoyl phosphatidylcholine (DMPC), dipalmitoyl phosphatidylcholine (DPPC), and distearyl phosphatidylcholine (DSPC), and (iii) a phosphatidylglycerol phospholipid (PG) selected from the group consisting of dimyristoyl phosphatidylglycerol (DMPG), dipalmitoyl phosphatidylglycerol (DPPG), and distearyl phosphatidylglycerol (DSPG). In a particular embodiment, a liposomal formulation comprises a TLR 7/8 modulating molecule described herein, DMPC, and DMPG.

In some embodiments, a liposomal formulation comprises a TLR 7/8 modulating molecule described herein and phospholipids, wherein the hydrocarbon chains of said phospholipids have a melting temperature in water of ≥23° C. In some embodiments, a liposomal formulation comprises a TLR 7/8 modulating molecule described herein and phospholipids DMPC and DMPG, wherein the hydrocarbon chains of said phospholipids have a melting temperature in water of ≥23° C. In some embodiments, a liposomal formulation comprises a TLR 7/8 modulating molecule described herein, phospholipids DMPC and DMPG, cholesterol, and QS-21, wherein the hydrocarbon chains of said phospholipids have a melting temperature in water of ≥23° C.

In some embodiments, a liposomal formulation comprises a TLR 7/8 modulating molecule described herein, a PC phospholipid, and a PG phospholipid, wherein the ratio of the PC phospholipid to the PG phospholipid (mol/mol) is about 0.5:1, about 1:1, about 2:1, about 3:1, about 4:1, about 5:1, about 6:1, about 7:1, about 8:1, about 9:1, about 10:1, about 11:1, about 12:1, about 13:1, about 14:1, or about 15:1. In some embodiments, a liposomal formulation comprises a TLR 7/8 modulating molecule described herein, DMPC, and DMPG, wherein the ratio of the DMPC to the DMPG (mol/mol) is about 0.5:1, about 1:1, about 2:1, about 3:1, about 4:1, about 5:1, about 6:1, about 7:1, about 8:1, about 9:1, about 10:1, about 11:1, about 12:1, about 13:1, about 14:1, or about 15:1. In some embodiments, a liposomal formulation comprises a TLR 7/8 modulating molecule described herein, a PC phospholipid, and a PG phospholipid, wherein the ratio of the PC phospholipid to the PG phospholipid (mol/mol) is between about 8:1 and about 10:1. In some embodiments, a liposomal formulation comprises a TLR 7/8 modulating molecule described herein, DMPC, and DMPG, wherein the ratio of the DMPC to the DMPG (mol/mol) is between about 8:1 and about 10:1. In some embodiments, a liposomal formulation comprises a TLR 7/8 modulating molecule described herein, a PC phospholipid, and a PG phospholipid, wherein the ratio of the PC phospholipid to the PG phospholipid (mol/mol) is about 9:1. In some embodiments, a liposomal formulation comprises a TLR 7/8 modulating molecule described herein, DMPC, and DMPG, wherein the ratio of the DMPC to the DMPG (mol/mol) is about 9:1. In some embodiments, a liposomal formulation comprises a TLR 7/8 modulating molecule described herein, DMPC, DMPG, cholesterol, and QS-21, wherein the ratio of the DMPC to the DMPG (mol/mol) is about 9:1.

In some embodiments, a liposomal formulation comprises a TLR 7/8 modulating molecule described herein and a PC phospholipid, wherein the concentration of the PC phospholipid is between about 1 mg/mL and about 100 mg/mL. In some embodiments, a liposomal formulation comprises a TLR 7/8 modulating molecule described herein and DMPC, wherein the concentration of the DMPC is between about 1 mg/ml and about 100 mg/mL. In some embodiments, a liposomal formulation comprises a TLR 7/8 modulating molecule described herein and a PC phospholipid, wherein the concentration of the PC phospholipid is between about 10 mg/mL and about 50 mg/mL. In some embodiments, a liposomal formulation comprises a TLR 7/8 modulating molecule described herein and DMPC, wherein the concentration of the DMPC is between about 10 mg/mL and about 50 mg/mL. In some embodiments, a liposomal formulation comprises a TLR 7/8 modulating molecule described herein and a PC phospholipid, wherein the concentration of the PC phospholipid is between about 20 mg/mL and about 30 mg/mL. In some embodiments, a liposomal formulation comprises a TLR 7/8 modulating molecule described herein and DMPC, wherein the concentration of the DMPC is between about 20 mg/mL and about 30 mg/mL. In some embodiments, a liposomal formulation comprises a TLR 7/8 modulating molecule described herein and a PC phospholipid, wherein the concentration of the PC phospholipid is about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, or about 35 mg/mL. In some embodiments, a liposomal formulation comprises a TLR 7/8 modulating molecule described herein and DMPC, wherein the concentration of the DMPC is about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, or about 35 mg/mL. In some embodiments, a liposomal formulation comprises a TLR 7/8 modulating molecule described herein and a PC phospholipid, wherein the concentration of the PC phospholipid is about 28 mg/mL. In a preferred embodiment, a liposomal formulation comprises a TLR 7/8 modulating molecule described herein and DMPC, wherein the concentration of the DMPC is about 28 mg/mL. In another preferred embodiment, a liposomal formulation comprises a TLR 7/8 modulating molecule described herein, DMPC, DMPG, cholesterol, and QS-21, wherein the concentration of the DMPC is about 28 mg/mL.

In some embodiments, a liposomal formulation comprises a TLR 7/8 modulating molecule described herein and a PC phospholipid, wherein the concentration of the PC phospholipid is between about 1 mg/mL and about 5 mg/mL. In some embodiments, a liposomal formulation comprises a TLR 7/8 modulating molecule described herein and DMPC, wherein the concentration of the DMPC is between about 1 mg/mL and about 5 mg/ml. In some embodiments, a liposomal formulation comprises a TLR 7/8 modulating molecule described herein and a PC phospholipid, wherein the concentration of the PC phospholipid is between about 2 mg/mL and about 3 mg/mL. In some embodiments, a liposomal formulation comprises a TLR 7/8 modulating molecule described herein and DMPC, wherein the concentration of the DMPC is between about 2 mg/mL and about 3 mg/mL. In some embodiments, a liposomal formulation comprises a TLR 7/8 modulating molecule described herein and a PC phospholipid, wherein the concentration of the PC phospholipid is about 2 mg/ml, about 2.1 mg/ml, about 2.2 mg/mL, about 2.3 mg/mL, about 2.4 mg/mL, about 2.5 mg/mL, about 2.6 mg/mL, about 2.7 mg/mL, about 2.8 mg/mL, about 2.9 mg/mL, about 3.0 mg/mL, about 3.1 mg/mL, about 3.2 mg/mL, about 3.3 mg/mL, about 3.4 mg/mL, or about 3.5 mg/mL. In some embodiments, a liposomal formulation comprises a TLR 7/8 modulating molecule described herein and DMPC, wherein the concentration of the DMPC is about 2.0 mg/mL, about 2.1 mg/mL, about 2.2 mg/ml, about 2.3 mg/mL, about 2.4 mg/mL, about 2.5 mg/mL, about 2.6 mg/mL, about 2.7 mg/mL, about 2.8 mg/mL, about 2.9 mg/mL, about 3.0 mg/mL, about 3.1 mg/mL, about 3.2 mg/ml, about 3.3 mg/mL, about 3.4 mg/mL, or about 3.5 mg/mL. In some embodiments, a liposomal formulation comprises a TLR 7/8 modulating molecule described herein and a PC phospholipid, wherein the concentration of the PC phospholipid is about 2.8 mg/mL. In a preferred embodiment, a liposomal formulation comprises a TLR 7/8 modulating molecule described herein and DMPC, wherein the concentration of the DMPC is about 2.8 mg/mL. In another preferred embodiment, a liposomal formulation comprises a TLR 7/8 modulating molecule described herein, DMPC, DMPG, cholesterol, and QS-21, wherein the concentration of the DMPC is about 2.8 mg/mL.

In some embodiments, a liposomal formulation comprises a TLR 7/8 modulating molecule described herein and a PC phospholipid, wherein the concentration of the PC phospholipid is between about 5 mg/mL and about 25 mg/mL. In some embodiments, a liposomal formulation comprises a TLR 7/8 modulating molecule described herein and DMPC, wherein the concentration of the DMPC is between about 5 mg/ml and about 25 mg/mL. In some embodiments, a liposomal formulation comprises a TLR 7/8 modulating molecule described herein and a PC phospholipid, wherein the concentration of the PC phospholipid is between about 10 mg/mL and about 15 mg/mL. In some embodiments, a liposomal formulation comprises a TLR 7/8 modulating molecule described herein and DMPC, wherein the concentration of the DMPC is between about 10 mg/mL and about 15 mg/mL. In some embodiments, a liposomal formulation comprises a TLR 7/8 modulating molecule described herein and a PC phospholipid, wherein the concentration of the PC phospholipid is about 10 mg/ml, about 10.5 mg/mL, about 11 mg/mL, about 11.5 mg/mL, about 12 mg/mL, about 12.5 mg/mL, about 13 mg/mL, about 13.5 mg/mL, about 14 mg/mL, about 14.5 mg/mL, about 15 mg/mL, about 15.5 mg/mL, about 16 mg/mL, about 16.5 mg/mL, about 17 mg/mL, or about 17.5 mg/mL. In some embodiments, a liposomal formulation comprises a TLR 7/8 modulating molecule described herein and DMPC, wherein the concentration of the DMPC is about 10 mg/mL, about 10.5 mg/mL, about 11 mg/mL, about 11.5 mg/mL, about 12 mg/mL, about 12.5 mg/mL, about 13 mg/mL, about 13.5 mg/mL, about 14 mg/mL, about 14.5 mg/mL, about 15 mg/mL, about 15.5 mg/mL, about 16 mg/mL, about 16.5 mg/mL, about 17 mg/mL, or about 17.5 mg/mL. In some embodiments, a liposomal formulation comprises a TLR 7/8 modulating molecule described herein and a PC phospholipid, wherein the concentration of the PC phospholipid is about 14 mg/mL. In a preferred embodiment, a liposomal formulation comprises a TLR 7/8 modulating molecule described herein and DMPC, wherein the concentration of the DMPC is about 14 mg/mL. In another preferred embodiment, a liposomal formulation comprises a TLR 7/8 modulating molecule described herein, DMPC, DMPG, cholesterol, and QS-21, wherein the concentration of the DMPC is about 14 mg/mL.

In some embodiments, a liposomal formulation comprises a TLR 7/8 modulating molecule described herein and a PC phospholipid, wherein the concentration of the PC phospholipid is between about 20 mg/mL and about 100 mg/mL. In some embodiments, a liposomal formulation comprises a TLR 7/8 modulating molecule described herein and DMPC, wherein the concentration of the DMPC is between about 20 mg/mL and about 100 mg/ml. In some embodiments, a liposomal formulation comprises a TLR 7/8 modulating molecule described herein and a PC phospholipid, wherein the concentration of the PC phospholipid is between about 40 mg/mL and about 60 mg/mL. In some embodiments, a liposomal formulation comprises a TLR 7/8 modulating molecule described herein and DMPC, wherein the concentration of the DMPC is between about 40 mg/mL and about 60 mg/mL. In some embodiments, a liposomal formulation comprises a TLR 7/8 modulating molecule described herein and a PC phospholipid, wherein the concentration of the PC phospholipid is about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, about 50 mg/mL, about 51 mg/mL, about 52 mg/mL, about 53 mg/mL, about 54 mg/mL, about 55 mg/mL, about 56 mg/mL, about 57 mg/mL, about 58 mg/mL, about 59 mg/mL or about 60 mg/mL. In some embodiments, a liposomal formulation comprises a TLR 7/8 modulating molecule described herein and DMPC, wherein the concentration of the DMPC is about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, about 50 mg/mL, about 51 mg/mL, about 52 mg/mL, about 53 mg/mL, about 54 mg/mL, about 55 mg/mL, about 56 mg/mL, about 57 mg/mL, about 58 mg/mL, about 59 mg/ml or about 60 mg/mL. In some embodiments, a liposomal formulation comprises a TLR 7/8 modulating molecule described herein and a PC phospholipid, wherein the concentration of the PC phospholipid is about 56 mg/mL. In a preferred embodiment, a liposomal formulation comprises a TLR 7/8 modulating molecule described herein and DMPC, wherein the concentration of the DMPC is about 56 mg/mL. In another preferred embodiment, a liposomal formulation comprises a TLR 7/8 modulating molecule described herein, DMPC, DMPG, cholesterol, and QS-21, wherein the concentration of the DMPC is about 56 mg/mL.

In some embodiments, a liposomal formulation comprises a TLR 7/8 modulating molecule described herein and a PG phospholipid, wherein the concentration of the PG phospholipid is between about 0.1 mg/mL and about 50 mg/mL. In some embodiments, a liposomal formulation comprises a TLR 7/8 modulating molecule described herein and DMPG, wherein the concentration of the DMPG is between about 0.1 mg/ml and about 50 mg/ml. In some embodiments, a liposomal formulation comprises a TLR 7/8 modulating molecule described herein and a PG phospholipid, wherein the concentration of the PG phospholipid is between about 0.1 mg/mL and about 10 mg/mL. In some embodiments, a liposomal formulation comprises a TLR 7/8 modulating molecule described herein and DMPG, wherein the concentration of the DMPG is between about 0.1 mg/mL and about 10 mg/mL. In some embodiments, a liposomal formulation comprises a TLR 7/8 modulating molecule described herein and a PG phospholipid, wherein the concentration of the PG phospholipid is about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, or about 10 mg/mL. In some embodiments, a liposomal formulation comprises a TLR 7/8 modulating molecule described herein and DMPG, wherein the concentration of the DMPG is about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, or about 10 mg/mL. In some embodiments, a liposomal formulation comprises a TLR 7/8 modulating molecule described herein and a PG phospholipid, wherein the concentration of the PG phospholipid is between about 3 mg/mL and about 4 mg/mL. In some embodiments, a liposomal formulation comprises a TLR 7/8 modulating molecule described herein and DMPG, wherein the concentration of the DMPG is between about 3 mg/ml and about 4 mg/mL. In some embodiments, a liposomal formulation comprises a TLR 7/8 modulating molecule described herein and a PG phospholipid, wherein the concentration of the PG phospholipid is about 3 mg/mL, about 3.1 mg/mL, about 3.2 mg/mL, about 3.3 mg/mL, about 3.4 mg/mL, about 3.5 mg/mL, about 3.6 mg/mL, about 3.7 mg/mL, about 3.8 mg/mL, about 3.9 mg/mL, or about 4 mg/mL. In some embodiments, a liposomal formulation comprises a TLR 7/8 modulating molecule described herein and DMPG, wherein the concentration of the DMPG is about 3 mg/ml, about 3.1 mg/mL, about 3.2 mg/mL, about 3.3 mg/mL, about 3.4 mg/mL, about 3.5 mg/mL, about 3.6 mg/mL, about 3.7 mg/mL, about 3.8 mg/mL, about 3.9 mg/mL, or about 4 mg/mL. In some embodiments, a liposomal formulation comprises a TLR 7/8 modulating molecule described herein and a PG phospholipid, wherein the concentration of the PG is about 3.2 mg/mL. In a particular embodiment, a liposomal formulation comprises a TLR 7/8 modulating molecule described herein and DMPG, wherein the concentration of the DMPG is about 3.2 mg/mL. In another particular embodiment, a liposomal formulation comprises a TLR 7/8 modulating molecule described herein, DMPG, DMPC, cholesterol, and QS-21, wherein the concentration of the DMPG is about 3.2 mg/mL.

In some embodiments, a liposomal formulation comprises a TLR 7/8 modulating molecule described herein and a PG phospholipid, wherein the concentration of the PG phospholipid is about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, or about 1.0 mg/mL. In some embodiments, a liposomal formulation comprises a TLR 7/8 modulating molecule described herein and DMPG, wherein the concentration of the DMPG is about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, or about 1.0 mg/mL. In some embodiments, a liposomal formulation comprises a TLR 7/8 modulating molecule described herein and a PG phospholipid, wherein the concentration of the PG phospholipid is between about 0.3 mg/mL and about 0.4 mg/mL. In some embodiments, a liposomal formulation comprises a TLR 7/8 modulating molecule described herein and DMPG, wherein the concentration of the DMPG is between about 0.3 mg/ml and about 0.4 mg/mL. In some embodiments, a liposomal formulation comprises a TLR 7/8 modulating molecule described herein and a PG phospholipid, wherein the concentration of the PG phospholipid is about 0.3 mg/mL, about 0.31 mg/mL, about 0.32 mg/mL, about 0.33 mg/mL, about 0.34 mg/mL, about 0.35 mg/mL, about 0.36 mg/mL, about 0.37 mg/mL, about 0.38 mg/mL, about 0.39 mg/mL, or about 0.4 mg/mL. In some embodiments, a liposomal formulation comprises a TLR 7/8 modulating molecule described herein and DMPG, wherein the concentration of the DMPG is about 0.3 mg/mL, about 0.31 mg/mL, about 0.32 mg/mL, about 0.33 mg/mL, about 0.34 mg/mL, about 0.35 mg/mL, about 0.36 mg/mL, about 0.37 mg/mL, about 0.38 mg/mL, about 0.39 mg/mL, or about 0.4 mg/mL. In some embodiments, a liposomal formulation comprises a TLR 7/8 modulating molecule described herein and a PG phospholipid, wherein the concentration of the PG is about 0.32 mg/mL. In a particular embodiment, a liposomal formulation comprises a TLR 7/8 modulating molecule described herein and DMPG, wherein the concentration of the DMPG is about 0.32 mg/mL. In another particular embodiment, a liposomal formulation comprises a TLR 7/8 modulating molecule described herein, DMPG, DMPC, cholesterol, and QS-21, wherein the concentration of the DMPG is about 0.32 mg/mL.

In some embodiments, a liposomal formulation comprises a TLR 7/8 modulating molecule described herein and a PG phospholipid, wherein the concentration of the PG phospholipid is about 0.5 mg/mL, about 0.6 mg/mL, about 0.7 mg/mL, about 0.8 mg/mL, about 0.9 mg/mL, about 1 mg/mL, about 1.1 mg/mL, about 1.2 mg/mL, about 1.3 mg/mL, about 1.4 mg/mL, about 1.5 mg/mL, about 1.6 mg/mL, about 1.7 mg/mL, about 1.8 mg/mL, about 1.9 mg/mL, or about 2 mg/mL. In some embodiments, a liposomal formulation comprises a TLR 7/8 modulating molecule described herein and DMPG, wherein the concentration of the DMPG is about 0.5 mg/mL, about 0.6 mg/mL, about 0.7 mg/mL, about 0.8 mg/mL, about 0.9 mg/mL, about 1 mg/mL, about 1.1 mg/mL, about 1.2 mg/mL, about 1.3 mg/mL, about 1.4 mg/mL, about 1.5 mg/mL, about 1.6 mg/mL, about 1.7 mg/mL, about 1.8 mg/mL, about 1.9 mg/mL, or about 2 mg/mL. In some embodiments, a liposomal formulation comprises a TLR 7/8 modulating molecule described herein and a PG phospholipid, wherein the concentration of the PG phospholipid is between about 1 mg/mL and about 2 mg/mL. In some embodiments, a liposomal formulation comprises a TLR 7/8 modulating molecule described herein and DMPG, wherein the concentration of the DMPG is between about 1 mg/mL and about 2 mg/mL. In some embodiments, a liposomal formulation comprises a TLR 7/8 modulating molecule described herein and a PG phospholipid, wherein the concentration of the PG is about 1.6 mg/mL. In a particular embodiment, a liposomal formulation comprises a TLR 7/8 modulating molecule described herein and DMPG, wherein the concentration of the DMPG is about 1.6 mg/mL. In another particular embodiment, a liposomal formulation comprises a TLR 7/8 modulating molecule described herein, DMPG, DMPC, cholesterol, and QS-21, wherein the concentration of the DMPG is about 1.6 mg/mL.

In some embodiments, a liposomal formulation comprises a TLR 7/8 modulating molecule described herein and a PG phospholipid, wherein the concentration of the PG phospholipid is about 6 mg/mL, about 6.1 mg/mL, about 6.2 mg/mL, about 6.3 mg/mL, about 6.4 mg/mL, about 6.5 mg/mL, about 6.6 mg/mL, about 6.7 mg/mL, about 6.8 mg/mL, about 6.9 mg/mL, or about 7 mg/mL. In some embodiments, a liposomal formulation comprises a TLR 7/8 modulating molecule described herein and DMPG, wherein the concentration of the DMPG is about 6 mg/mL, about 6.1 mg/mL, about 6.2 mg/mL, about 6.3 mg/mL, about 6.4 mg/mL, about 6.5 mg/mL, about 6.6 mg/mL, about 6.7 mg/mL, about 6.8 mg/mL, about 6.9 mg/mL, or about 7 mg/mL. In some embodiments, a liposomal formulation comprises a TLR 7/8 modulating molecule described herein and a PG phospholipid, wherein the concentration of the PG phospholipid is between about 6 mg/mL and about 7 mg/mL. In some embodiments, a liposomal formulation comprises a TLR 7/8 modulating molecule described herein and DMPG, wherein the concentration of the DMPG is between about 6 mg/mL and about 7 mg/mL. In some embodiments, a liposomal formulation comprises a TLR 7/8 modulating molecule described herein and a PG phospholipid, wherein the concentration of the PG is about 6.4 mg/mL. In a particular embodiment, a liposomal formulation comprises a TLR 7/8 modulating molecule described herein and DMPG, wherein the concentration of the DMPG is about 6.4 mg/mL. In another particular embodiment, a liposomal formulation comprises a TLR 7/8 modulating molecule described herein, DMPG, DMPC, cholesterol, and QS-21, wherein the concentration of the DMPG is about 6.4 mg/mL.

In some embodiments, a liposomal formulation comprises a TLR 7/8 modulating molecule described herein and cholesterol. In some embodiments, a liposomal formulation comprises a TLR 7/8 modulating molecule described herein and a cholesterol analog. In some embodiments, a liposomal formulation comprises a TLR 7/8 modulating molecule described herein, cholesterol, and a cholesterol analog. In some embodiments, a liposomal formulation comprises a TLR 7/8 modulating molecule described herein and a cholesterol analog selected from the group consisting of sitosterol, stigmasterol, campesterol, sitostanol, campestanol, brassicasterol, fucosterol, β-sitosterol, stigmastanol, β-sitostanol, ergosterol, fecosterol, lupeol, cycloartenol, Δ5-avenasterol, Δ7-avenasterol, Δ7-stigmasterol, tomatidine, ursolic acid, and alpha-tocopherol, including analogs, salts or esters thereof. In some embodiments, a liposomal formulation comprises a TLR 7/8 modulating molecule described herein, cholesterol, and a cholesterol analog selected from the group consisting of sitosterol, stigmasterol, campesterol, sitostanol, campestanol, brassicasterol, fucosterol, β-sitosterol, stigmastanol, β-sitostanol, ergosterol, fecosterol, lupeol, cycloartenol, Δ5-avenasterol, Δ7-avenasterol, Δ7-stigmasterol, tomatidine, ursolic acid, and alpha-tocopherol, including analogs, salts or esters thereof. In some embodiments, a liposomal formulation comprises a TLR 7/8 modulating molecule described herein, cholesterol, and β-sitosterol.

In some embodiments, a liposomal formulation comprises a TLR 7/8 modulating molecule described herein and cholesterol, wherein the concentration of cholesterol is between about 1 mg/mL and about 100 mg/mL. In some embodiments, a liposomal formulation comprises a TLR 7/8 modulating molecule described herein and cholesterol, wherein the concentration of cholesterol is between about 5 mg/mL and about 50 mg/mL. In some embodiments, a liposomal formulation comprises a TLR 7/8 modulating molecule described herein and cholesterol, wherein the concentration of cholesterol is between about 15 mg/ml and about 30 mg/ml. In some embodiments, a liposomal formulation comprises a TLR 7/8 modulating molecule described herein and cholesterol, wherein the concentration of cholesterol is about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, or about 30 mg/mL. In some embodiments, a liposomal formulation comprises a TLR 7/8 modulating molecule described herein and cholesterol, wherein the concentration of cholesterol is between about 21 mg/mL and about 22 mg/mL. In some embodiments, a liposomal formulation comprises a TLR 7/8 modulating molecule described herein and cholesterol, wherein the concentration of cholesterol is about 21 mg/mL, about 21.1 mg/mL, about 21.2 mg/mL, about 21.3 mg/mL, about 21.4 mg/mL, about 21.5 mg/mL, about 21.6 mg/mL, about 21.7 mg/mL, about 21.8 mg/mL, about 21.9 mg/mL, or about 22 mg/mL. In a preferred embodiment, a liposomal formulation comprises a TLR 7/8 modulating molecule described herein and cholesterol, wherein the concentration of cholesterol is about 21.6 mg/mL. In another preferred embodiment, a liposomal formulation comprises a TLR 7/8 modulating molecule described herein, cholesterol, DMPC, DMPG, and QS-21, wherein the concentration of cholesterol is about 21.6 mg/mL.

In some embodiments, a liposomal formulation comprises a TLR 7/8 modulating molecule described herein and cholesterol, wherein the concentration of cholesterol is between about 1 mg/mL and about 5 mg/mL. In some embodiments, a liposomal formulation comprises a TLR 7/8 modulating molecule described herein and cholesterol, wherein the concentration of cholesterol is about 1.5 mg/mL, about 1.6 mg/mL, about 1.7 mg/mL, about 1.8 mg/mL, about 1.9 mg/mL, about 2.0 mg/mL, about 2.1 mg/mL, about 2.2 mg/mL, about 2.3 mg/ml, about 2.4 mg/mL, about 2.5 mg/mL, about 2.6 mg/mL, about 2.7 mg/mL, about 2.8 mg/mL, about 2.9 mg/mL, or about 3.0 mg/mL. In some embodiments, a liposomal formulation comprises a TLR 7/8 modulating molecule described herein and cholesterol, wherein the concentration of cholesterol is between about 2.1 mg/mL and about 2.2 mg/mL. In some embodiments, a liposomal formulation comprises a TLR 7/8 modulating molecule described herein and cholesterol, wherein the concentration of cholesterol is about 2.1 mg/mL, about 2.11 mg/mL, about 2.12 mg/mL, about 2.13 mg/mL, about 2.14 mg/mL, about 2.15 mg/mL, about 2.16 mg/mL, about 2.17 mg/mL, about 2.18 mg/mL, about 2.19 mg/mL, or about 2.2 mg/mL. In a preferred embodiment, a liposomal formulation comprises a TLR 7/8 modulating molecule described herein and cholesterol, wherein the concentration of cholesterol is about 2.16 mg/mL. In another preferred embodiment, a liposomal formulation comprises a TLR 7/8 modulating molecule described herein, cholesterol, DMPC, DMPG, and QS-21, wherein the concentration of cholesterol is about 2.16 mg/mL.

In some embodiments, a liposomal formulation comprises a TLR 7/8 modulating molecule described herein and cholesterol, wherein the concentration of cholesterol is between about 5 mg/mL and about 20 mg/mL. In some embodiments, a liposomal formulation comprises a TLR 7/8 modulating molecule described herein and cholesterol, wherein the concentration of cholesterol is about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL. In some embodiments, a liposomal formulation comprises a TLR 7/8 modulating molecule described herein and cholesterol, wherein the concentration of cholesterol is between about 10 mg/mL and about 11 mg/mL. In some embodiments, a liposomal formulation comprises a TLR 7/8 modulating molecule described herein and cholesterol, wherein the concentration of cholesterol is about 10 mg/mL, about 10.1 mg/mL, about 10.2 mg/mL, about 10.3 mg/mL, about 10.4 mg/mL, about 10.5 mg/mL, about 10.6 mg/mL, about 10.7 mg/mL, about 10.8 mg/mL, about 10.9 mg/mL, or about 11 mg/mL. In a preferred embodiment, a liposomal formulation comprises a TLR 7/8 modulating molecule described herein and cholesterol, wherein the concentration of cholesterol is about 10.8 mg/mL. In another preferred embodiment, a liposomal formulation comprises a TLR 7/8 modulating molecule described herein, cholesterol, DMPC, DMPG, and QS-21, wherein the concentration of cholesterol is about 10.8 mg/mL.

In some embodiments, a liposomal formulation comprises a TLR 7/8 modulating molecule described herein and cholesterol, wherein the concentration of cholesterol is between about 40 mg/mL and about 50 mg/mL. In some embodiments, a liposomal formulation comprises a TLR 7/8 modulating molecule described herein and cholesterol, wherein the concentration of cholesterol is about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, or about 50 mg/mL. In some embodiments, a liposomal formulation comprises a TLR 7/8 modulating molecule described herein and cholesterol, wherein the concentration of cholesterol is between about 43 mg/mL and about 44 mg/mL. In some embodiments, a liposomal formulation comprises a TLR 7/8 modulating molecule described herein and cholesterol, wherein the concentration of cholesterol is about 43 mg/mL, about 43.1 mg/mL, about 43.2 mg/mL, about 43.3 mg/mL, about 43.4 mg/mL, about 43.5 mg/mL, about 43.6 mg/mL, about 43.7 mg/mL, about 43.8 mg/mL, about 43.9 mg/mL, or about 44 mg/mL. In a preferred embodiment, a liposomal formulation comprises a TLR 7/8 modulating molecule described herein and cholesterol, wherein the concentration of cholesterol is about 43.2 mg/mL. In another preferred embodiment, a liposomal formulation comprises a TLR 7/8 modulating molecule described herein, cholesterol, DMPC, DMPG, and QS-21, wherein the concentration of cholesterol is about 43.2 mg/mL.

In a preferred embodiment, a liposomal formulation comprises a TLR 7/8 modulating molecule described herein, DMPC, DMPG, and cholesterol, wherein the concentration of the compound in the liposomal formulation is about 0.0034 mg/mL, the concentration of the DMPC is about 2.8 mg/mL, the concentration of the DMPG is about 0.32 mg/mL, and the concentration of the cholesterol is about 2.16 mg/mL.

In another preferred embodiment, a liposomal formulation comprises a TLR 7/8 modulating molecule described herein, DMPC, DMPG, and cholesterol, wherein the concentration of the compound in the liposomal formulation is about 0.0172 mg/mL, the concentration of the DMPC is about 14 mg/mL, the concentration of the DMPG is about 1.6 mg/mL, and the concentration of the cholesterol is about 10.8 mg/mL.

In another preferred embodiment, a liposomal formulation comprises a TLR 7/8 modulating molecule described herein, DMPC, DMPG, and cholesterol, wherein the concentration of the compound in the liposomal formulation is about 0.0344 mg/mL, the concentration of the DMPC is about 28 mg/mL, the concentration of the DMPG is about 3.2 mg/mL, and the concentration of the cholesterol is about 21.6 mg/mL.

In another preferred embodiment, a liposomal formulation comprises a TLR 7/8 modulating molecule described herein, DMPC, DMPG, and cholesterol, wherein the concentration of the compound in the liposomal formulation is about 0.0688 mg/mL, the concentration of the DMPC is about 56 mg/mL, the concentration of the DMPG is about 6.4 mg/mL, and the concentration of the cholesterol is about 43.2 mg/mL.

In some embodiments, a liposomal formulation comprises a TLR 7/8 modulating molecule described herein, cholesterol, and phospholipids, wherein the mole ratio of cholesterol to phospholipids is between about 0.5:1 and about 5:1. In some embodiments, a liposomal formulation comprises a TLR 7/8 modulating molecule described herein, cholesterol, and phospholipids, wherein the mole ratio of cholesterol to phospholipids is between about 0.5:1 and about 5:1, and wherein the phospholipids are DMPC and DMPG. In some embodiments, a liposomal formulation comprises a TLR 7/8 modulating molecule described herein, cholesterol, and phospholipids, wherein the mole ratio of cholesterol to phospholipids is between about 1:1 and about 2:1. In some embodiments, a liposomal formulation comprises a TLR 7/8 modulating molecule described herein, cholesterol, and phospholipids, wherein the mole ratio of cholesterol to phospholipids is between about 1:1 and about 2:1, and wherein the phospholipids are DMPC and DMPG. In some embodiments, a liposomal formulation comprises a TLR 7/8 modulating molecule described herein, cholesterol, and phospholipids, wherein the mole ratio of cholesterol to phospholipids is about 55:30, about 55:35, about 55:40, about 55:45, about 55:50, about 1:1, about 55:60, about 55:65, or about 55:70. In some embodiments, a liposomal formulation comprises a TLR 7/8 modulating molecule described herein, cholesterol, and phospholipids, wherein the mole ratio of cholesterol to phospholipids is about 55:30, about 55:35, about 55:40, about 55:45, about 55:50, about 1:1, about 55:60, about 55:65, or about 55:70, and wherein the phospholipids are DMPC and DMPG. In some embodiments, a liposomal formulation comprises a TLR 7/8 modulating molecule described herein, cholesterol, and phospholipids, wherein the mole ratio of cholesterol to phospholipids is about 55:45. In some embodiments, a liposomal formulation comprises a TLR 7/8 modulating molecule described herein, cholesterol, and phospholipids, wherein the mole ratio of cholesterol to the combination of DMPC and DMPG is about 55:45. In some embodiments, a liposomal formulation comprises a TLR 7/8 modulating molecule described herein, cholesterol, DMPC, DMPG, and QS-21, wherein the mole ratio of cholesterol to the combination of DMPC and DMPG is about 55:45.

In one embodiment, the liposomal adjuvant formulation is Adjuvant 1 (A1). In one embodiment, a 1X concentration of A1 comprises a TLR 7/8 modulating molecule described herein, cholesterol, DMPC, DMPG, and QS-21, wherein the concentration of QS-21 is about 0.1 mg/mL, the concentration of the TLR 7/8 modulating molecule is between about 0.06 mg/ml and about 0.07 mg/mL, the concentration of DMPC is about 28 mg/mL, the concentration of DMPG is between about 3.1 mg/mL and about 3.3 mg/mL, and the concentration of cholesterol is between about 21 mg/mL and about 22 mg/mL. In one embodiment, a 1X concentration of A1 comprises a TLR 7/8 modulating molecule described herein, cholesterol, DMPC, DMPG, and QS-21, wherein the concentration of QS-21 is about 0.2 mg/mL, the concentration of the TLR 7/8 modulating molecule is between about 0.06 mg/mL and about 0.07 mg/mL, the concentration of DMPC is about 28 mg/mL, the concentration of DMPG is between about 3.1 mg/ml and about 3.3 mg/mL, and the concentration of cholesterol is between about 21 mg/mL and about 22 mg/mL. In one embodiment, a 1X concentration of A1 comprises a TLR 7/8 modulating molecule described herein, cholesterol, DMPC, DMPG, and QS-21, wherein the concentration of QS-21 is about 0.3 mg/mL, the concentration of the TLR 7/8 modulating molecule is between about 0.06 mg/mL and about 0.07 mg/mL, the concentration of DMPC is about 28 mg/mL, the concentration of DMPG is between about 3.1 mg/mL and about 3.3 mg/mL, and the concentration of cholesterol is between about 21 mg/mL and about 22 mg/mL. In one embodiment, a 1X concentration of A1 comprises a TLR 7/8 modulating molecule described herein, cholesterol, DMPC, DMPG, and QS-21, wherein the concentration of QS-21 is about 0.4 mg/mL, the concentration of the TLR 7/8 modulating molecule is between about 0.06 mg/mL and about 0.07 mg/mL, the concentration of DMPC is about 28 mg/mL, the concentration of DMPG is between about 3.1 mg/ml and about 3.3 mg/mL, and the concentration of cholesterol is between about 21 mg/mL and about 22 mg/mL. In some embodiments, the A1 formulation is A1 at a 0.0625X concentration (0.0625X A1)(i.e., wherein each of DMPC, DMPG, cholesterol, QS-21, and the TLR 7/8 modulating molecule have a concentration 16 times lower than in 1X A1). In some embodiments, the A1 formulation is A1 at a 0. 125X concentration (0.125X A1). In some embodiments, the A1 formulation is A1 at a 0.25X concentration (0.25X A1). In some embodiments, the A1 formulation is A1 at a 0.5X concentration (0.5X A1). In some embodiments, the A1 formulation is A1 at a 2X concentration (2X A1). In some embodiments, the A1 formulation is A1 at a 3X concentration (3X A1). In some embodiments, the A1 formulation is A1 at a 4X concentration (4X A1). In some embodiments, the A1 formulation is A1 at a 8X concentration (8X A1).

In some embodiments, a liposomal formulation comprises a TLR 7/8 modulating molecule described herein, wherein the liposomal formulation comprises a buffer. In some embodiments, a liposomal formulation comprises a TLR 7/8 modulating molecule described herein, wherein the liposomal formulation comprises a phosphate buffer. In some embodiments, a liposomal formulation comprises a TLR 7/8 modulating molecule described herein, wherein the liposomal formulation comprises a phosphate buffer at a concentration between about 1 mM and about 100 mM. In some embodiments, a liposomal formulation comprises a TLR 7/8 modulating molecule described herein, wherein the liposomal formulation comprises a phosphate buffer at a concentration between about 5 mM and about 50 mM. In some embodiments, a liposomal formulation comprises a TLR 7/8 modulating molecule described herein, wherein the liposomal formulation comprises a phosphate buffer at a concentration of about 1 mM, about 5 mM, about 10 mM, about 15 mM, about 20 mM, about 25 mM, about 30 mM, about 35 mM, about 40 mM, about 45 mM, or about 50 mM. In a preferred embodiment, a liposomal formulation comprises a TLR 7/8 modulating molecule described herein, wherein the liposomal formulation comprises a phosphate buffer at a concentration of about 10 mM. In another preferred embodiment, a liposomal formulation comprises a TLR 7/8 modulating molecule described herein, wherein the liposomal formulation comprises DMPC, DMPG, cholesterol, QS-21, and a phosphate buffer at a concentration of about 10 mM.

In some embodiments, a liposomal formulation comprises a TLR 7/8 modulating molecule described herein, wherein the liposomal formulation comprises a buffer and wherein the pH of the buffer is between about 5 and about 8. In some embodiments, a liposomal formulation comprises a TLR 7/8 modulating molecule described herein, wherein the liposomal formulation comprises a buffer and wherein the pH of the buffer is about 5, about 5.5, about 6, about 6.5, about 7, about 7.5, or about 8. In some embodiments, a liposomal formulation comprises a TLR 7/8 modulating molecule described herein, wherein the liposomal formulation comprises a buffer and wherein the pH of the buffer is about 6, about 6.1, about 6.2, about 6.3, about 6.4, about 6.5, about 6.6, about 6.7, about 6.8, about 6.9, or about 7. In some embodiments, a liposomal formulation comprises a TLR 7/8 modulating molecule described herein, wherein the liposomal formulation comprises a phosphate buffer and wherein the pH of the buffer is about 6, about 6.1, about 6.2, about 6.3, about 6.4, about 6.5, about 6.6, about 6.7, about 6.8, about 6.9, or about 7. In a preferred embodiment, a liposomal formulation comprises a TLR 7/8 modulating molecule described herein, wherein the liposomal formulation comprises a buffer and wherein the pH of the buffer is about 6.2. In another preferred embodiment, a liposomal formulation comprises a TLR 7/8 modulating molecule described herein, wherein the liposomal formulation comprises a phosphate buffer, wherein the pH of the buffer is about 6.2 and the concentration of phosphate is about 10 mM. In another preferred embodiment, a liposomal formulation comprises a TLR 7/8 modulating molecule described herein, wherein the liposomal formulation comprises DMPC, DMPG, cholesterol, QS-21, and a phosphate buffer, wherein the pH of the buffer is about 6.2 and the concentration of phosphate is about 10 mM.

In some embodiments, a liposomal formulation comprises a TLR 7/8 modulating molecule described herein, wherein the liposomal formulation comprises a buffer and wherein the buffer comprises sodium chloride. In some embodiments, a liposomal formulation comprises a TLR 7/8 modulating molecule described herein, wherein the liposomal formulation comprises a buffer and wherein the buffer comprises sodium chloride at a concentration between about 10 mM and about 500 mM. In some embodiments, a liposomal formulation comprises a TLR 7/8 modulating molecule described herein, wherein the liposomal formulation comprises sodium chloride at a concentration between about 50 mM and about 250 mM. In some embodiments, a liposomal formulation comprises a TLR 7/8 modulating molecule described herein, wherein the liposomal formulation comprises a buffer and wherein the buffer comprises sodium chloride at a concentration between about 100 mM and about 200 mM. In some embodiments, a liposomal formulation comprises a TLR 7/8 modulating molecule described herein, wherein the liposomal formulation comprises a buffer and wherein the buffer comprises sodium chloride at a concentration of about 100 mM, about 110 mM, about 120 mM, about 130 mM, about 140 mM, about 150 mM, about 160 mM, about 170 mM, about 180 mM, about 190 mM, or about 200 mM. In some embodiments, a liposomal formulation comprises a TLR 7/8 modulating molecule described herein, wherein the liposomal formulation comprises a buffer and wherein the buffer comprises sodium chloride at a concentration of about 150 mM. In some embodiments, a liposomal formulation comprises a TLR 7/8 modulating molecule described herein, wherein the liposomal formulation comprises sodium chloride at a concentration of about 150 mM and phosphate at a concentration of about 10 mM. In a preferred embodiment, a liposomal formulation comprises a TLR 7/8 modulating molecule described herein, wherein the liposomal formulation comprises a buffer and wherein the buffer comprises sodium chloride at a concentration of about 150 mM and phosphate at a concentration of 10 mM, pH 6.2. In a preferred embodiment, a liposomal formulation comprises a TLR 7/8 modulating molecule described herein, wherein the liposomal formulation comprises DMPC, DMPG, cholesterol, QS-21, and a buffer and wherein the buffer comprises sodium chloride at a concentration of about 150 mM and phosphate at a concentration of about 10 mM, pH 6.2.

In some embodiments, a liposomal formulation comprises a TLR 7/8 modulating molecule described herein, wherein said liposomal formulation comprises a population of liposomes that are sized to nanoscale (i.e, have a size less than 1 micrometer). The diameter of the liposomes described herein can be determined using methods known within the art, such as dynamic light scattering (DLS)(See e.g., Stetefeld et al. Biophys Rev. 2016 December; 8 (4): 409-427), transmission electron microscopy (TEM)(See e.g., Keck et al. Int J Pharm. 2008 May 1; 355 (1-2): 150-63) and laser diffraction (See e.g., Filippov et al. Mater Horiz. 2023 Nov. 27; 10 (12): 5354-5370). As used herein, in reference to a population of particles, the particle size provided is the z-average diameter of the population of particles as measured by dynamic light scattering (DLS)(See e.g., Yeap et al. J Nanosci Nanotechnol. 2018 Oct. 1; 18 (10): 6957-6964). The term “mean diameter” refers to the mean hydrodynamic diameter of particles as measured by dynamic laser light scattering (DLS) with data analysis using the so-called cumulant algorithm, which provides as results the so-called Z-average with the dimension of a length, and the polydispersity index (PDI), which is dimensionless (Koppel, D., J. Chem. Phys. 57, 1972, pp 4814-4820, ISO 13321). Here, “mean diameter,” “diameter,” “size” or “mean size” for particles is used synonymously with this value of the Z-average.

In some embodiments, a liposomal formulation comprises a TLR 7/8 modulating molecule described herein, wherein said liposomal formulation comprises a population of liposomes, and wherein the average size of the liposomes is between about 1 nanometer (nm) and about 1 micrometer (μM) in diameter. In some embodiments, a liposomal formulation comprises a TLR 7/8 modulating molecule described herein, wherein said liposomal formulation comprises a population of liposomes, wherein the average size of the liposomes is about 1 nm, about 50 nm, about 100 nm, about 150 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, or about 1000 nm in diameter. In some embodiments, a liposomal formulation comprises a TLR 7/8 modulating molecule described herein, wherein said liposomal formulation comprises a population of liposomes, wherein the average size of the liposomes is between about 10 nm and about 500 nm in diameter. In some embodiments, a liposomal formulation comprises a TLR 7/8 modulating molecule described herein, wherein said liposomal formulation comprises a population of liposomes, wherein the average size of the liposomes is between about 10 nm and about 200 nm in diameter. In some embodiments, a liposomal formulation comprises a TLR 7/8 modulating molecule described herein, wherein said liposomal formulation comprises a population of liposomes, wherein the average size of the liposomes is between about 80 nm and about 200 nm in diameter. In some embodiments, a liposomal formulation comprises a TLR 7/8 modulating molecule described herein, wherein said liposomal formulation comprises a population of liposomes, wherein the average size of the liposomes is between about 100 nm and about 170 nm in diameter. In some embodiments, a liposomal formulation comprises a TLR 7/8 modulating molecule described herein, wherein said liposomal formulation comprises a population of liposomes, wherein the average size of the liposomes is about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, or about 170 nm in diameter. In some embodiments, a liposomal formulation comprises a TLR 7/8 modulating molecule described herein, DMPC, DMPG, cholesterol, and QS-21, wherein said liposomal formulation comprises a population of liposomes, wherein the average size of the liposomes is about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, or about 170 nm in diameter.

In some embodiments, a liposomal formulation comprises a TLR 7/8 modulating molecule described herein, wherein said liposomal formulation comprises a population of liposomes, wherein the average polydispersity index (PDI) of the liposomes is less than about 0.5. Polydispersity is measured by a polydispersity index (PDI). Calculations used for the determination of size and PDI parameters may be found in the ISO standard documents 13321:1996 E and ISO 22412:2008 (See Worldwide M.I. Dynamic Light Scattering, Common Terms Defined. Malvern Instruments Limited; Malvern, UK: 2011. Pp. 1-6. Inform White Paper). In some embodiments, a liposomal formulation comprises a TLR 7/8 modulating molecule described herein, wherein said liposomal formulation comprises a population of liposomes, wherein the PDI of the liposomes is about 0.1, about 0.2, about 0.3, about 0.4, or about 0.5. In some embodiments, a liposomal formulation comprises a TLR 7/8 modulating molecule described herein, wherein said liposomal formulation comprises a population of liposomes, wherein the PDI of the liposomes is less than about 0.3. In some embodiments, a liposomal formulation comprises a TLR 7/8 modulating molecule described herein, wherein said liposomal formulation comprises a population of liposomes, wherein the PDI of the liposomes is about 0.1, about 0.11, about 0.12, about 0.13, about 0.14, about 0.15, about 0.16, about 0.17, about 0.18, about 0.19, about 0.2, about 0.21, about 0.22, about 0.23, about 0.24, about 0.25, about 0.26, about 0.27, about 0.28, about 0.29, or about 0.3. In some embodiments, a liposomal formulation comprises a TLR 7/8 modulating molecule described herein, DMPC, DMPG, cholesterol, and QS-21, wherein said liposomal formulation comprises a population of liposomes, wherein the PDI of the liposomes is about 0.1, about 0.11, about 0.12, about 0.13, about 0.14, about 0.15, about 0.16, about 0.17, about 0.18, about 0.19, about 0.2, about 0.21, about 0.22, about 0.23, about 0.24, about 0.25, about 0.26, about 0.27, about 0.28, about 0.29, or about 0.3.

In some embodiments, the size of the liposomes comprised within the liposomal formulation is determined at time zero (T0), wherein TO is the time when the formation of the liposomes in the liposomal formulation is completed. In some embodiments, the PDI of the liposomes comprised within the liposomal formulation is determined at time zero (T0), wherein TO is the time when the formation of the liposomes in the liposomal formulation is completed.

In some embodiments, a liposomal formulation comprises a TLR 7/8 modulating molecule described herein, wherein said liposomal formulation comprises a population of liposomes, wherein the liposomes are stable as determined by maintaining about the same average size (nm) over the course of a given time period. In some embodiments, a liposomal formulation comprises a TLR 7/8 modulating molecule described herein, wherein said liposomal formulation comprises a population of liposomes, wherein the liposomes maintain an average size (nm) that deviates by no more than about 5%, about 10%, about 15%, about 20%, about 25%, about 30%, about 40%, or about 50% of the value of their average size at time zero (T0) over the course of at least about 5 days, about 10 days, about 15 days, about 20 days, about 25 days, about 30 days, about 35 days, about 40 days, about 45 days, about 50 days, about 55 days, about 60 days, about 65 days, about 70 days, about 75 days, about 80 days, about 85 days, about 90 days, about 95 days, or about 100 days after TO. In some embodiments, a liposomal formulation comprises a TLR 7/8 modulating molecule described herein, wherein said liposomal formulation comprises a population of liposomes, wherein the liposomes maintain an average size (nm) that deviates by no more than about 5%, about 10%, about 15%, about 20%, about 25%, about 30%, about 40%, or about 50% of the value of their average size at time zero (T0) over the course of at least about 1 month, about 2 months, about 3 months, about 4 months, about 5 months, about 6 months, about 7 months, about 8 months, about 9 months, about 10 months, about 11 months, or about 12 months after TO. In a particular embodiment, a liposomal formulation comprises a TLR 7/8 modulating molecule described herein, wherein said liposomal formulation comprises a population of liposomes, wherein the liposomes maintain an average size (nm) that deviates by no more than about 20% of the value of their average size at time zero (T0) for at least about 3 months after TO.

In some embodiments, a liposomal formulation comprises a TLR 7/8 modulating molecule described herein, wherein said liposomal formulation comprises a population of liposomes, wherein the liposomes are stable as determined by maintaining about the same PDI over the course of a given time period. In some embodiments, a liposomal formulation comprises a TLR 7/8 modulating molecule described herein, wherein said liposomal formulation comprises a population of liposomes, wherein the liposomes maintain a PDI that deviates by no more than about 5%, about 10%, about 15%, about 20%, about 25%, about 30%, about 35%, about 40%, about 45%, about 50%, about 55%, or about 60% of the value of their PDI at time zero (T0) over the course of at least about 5 days, about 10 days, about 15 days, about 20 days, about 25 days, about 30 days, about 35 days, about 40 days, about 45 days, about 50 days, about 55 days, about 60 days, about 65 days, about 70 days, about 75 days, about 80 days, about 85 days, about 90 days, about 95 days, or about 100 days after TO. In some embodiments, a liposomal formulation comprises a TLR 7/8 modulating molecule described herein, wherein said liposomal formulation comprises a population of liposomes, wherein the liposomes maintain a PDI that deviates by no more than about 5%, about 10%, about 15%, about 20%, about 25%, about 30%, about 35%, about 40%, about 45%, about 50% of the value of their PDI at time zero (T0) over the course of at least about 1 month, about 2 months, about 3 months, about 4 months, about 5 months, about 6 months, about 7 months, about 8 months, about 9 months, about 10 months, about 11 months, or about 12 months after TO. In a particular embodiment, a liposomal formulation comprises a TLR 7/8 modulating molecule described herein, wherein said liposomal formulation comprises a population of liposomes, wherein the liposomes maintain a PDI that deviates by no more than about 40% of the value of their PDI at time zero (T0) for at least about 3 months after TO.

In some embodiments, the liposomes provided herein are produced using a microfluidic device. As used herein, a “microfluidic device” refers to a device with at least one channel having micron-scale dimensions (i.e., a dimension less than 1 mm) for processing (i.e., flowing, mixing, etc.) a fluid sample. In some embodiments, the microfluidic device is a passive device that contains no moving parts and has no requirement for energy input other than the pressure used to drive fluid flow through the device. In some embodiments, the microfluidic device comprises an interaction chamber, wherein compounds within a composition collide to form nanoparticles.

In one aspect, the liposomes provided herein are produced using microfluidization. Microfluidization can be performed in a microfluidic device wherein fluids are forced to pass through microchannels under high-pressure (for example, between 500 and 30,000 psi)(See Kumar et al. Prev Nutr Food Sci. 2019 September; 24 (3): 225-234). The microfluidization process also involves the collision of various compounds in a fluid composition within an interaction chamber. In the interaction chamber, compounds within the fluid collide at high velocity to form stable particles at nanoscale.

In some embodiments, the liposomes provided herein are produced using a microfluidic device that is a Y-junction, T-junction, or coaxial microfluidic device. In one aspect, the microfluidic device an internal diameter size ranging from 300 μm to 1,000 μm. The microfluidic device forms liposomes by a semi-continuous flow process. Examples of microfluidic devices include, but are not limited to, Y-mixer from Precision Nanosystem, T-mixer from IDEX®, a coaxial mixer, or any other configuration where turbulent flow is generated. “Turbulent flow” shall mean a flow in which the fluid undergoes irregular fluctuations or mixing, which is in contrast to laminar flow in which the fluid moves in smooth paths or layers. In turbulent flow the speed of the fluid at a point is continuously undergoing changes in both magnitude and direction.

In one embodiment, a method is provided for producing a liposomal formulation, comprising a plurality of liposomes, wherein the liposomes comprise (a) a TLR 7/8 modulating molecule described herein, (b) a saponin, (c) a phospholipid or phospholipids selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), and distearyl phosphatidylglycerol (DSPG), and (d) cholesterol, wherein the method comprises the following steps:
(i) dissolving the phospholipid(s), cholesterol and the TLR 7/8 modulating molecule in an organic solvent to form an organic phase;
(ii) mixing the organic phase of step (i) into an aqueous phase, wherein the aqueous phase comprises a buffer or water, in a microfluidic device to form an intermediate liposome;
(iii) removing the organic solvent of the intermediate liposome of step (ii);
(iv) concentrating the intermediate liposome of step (iii);
(v) filtering the intermediate liposome of step (iv); and
(vi) aseptically mixing the intermediate liposome of step (v) and the saponin.

In some embodiments of the method for producing a liposomal formulation, the phospholipids of step (i) comprise DMPC. In some embodiments of the method for producing a liposomal formulation, the phospholipids of step (i) comprise DMPG. In some embodiments of the method for producing a liposomal formulation, the phospholipids of step (i) comprise DMPC and DMPG.

In some embodiments of the method for producing a liposomal formulation, the aqueous phase of step (ii) comprises a phosphate buffer. In some embodiments of the method for producing a liposomal formulation, the aqueous phase of step (ii) comprises a phosphate buffer at a concentration between about 1 mM and about 100 mM. In some embodiments of the method for producing a liposomal formulation, the aqueous phase of step (ii) comprises a phosphate buffer at a concentration between about 1 mM and about 20 mM. In some embodiments of the method for producing a liposomal formulation, the aqueous phase of step (ii) comprises a phosphate buffer at a concentration of about 10 mM. In some embodiments of the method for producing a liposomal formulation, the aqueous phase of step (ii) comprises sodium chloride. In some embodiments of the method for producing a liposomal formulation, the aqueous phase of step (ii) comprises sodium chloride at a concentration between about 10 mM and about 500 mM. In some embodiments of the method for producing a liposomal formulation, the aqueous phase of step (ii) comprises a sodium chloride at a concentration between about 100 mM and about 200 mM. In some embodiments of the method for producing a liposomal formulation, the aqueous phase of step (ii) comprises a sodium chloride at a concentration of about 150 mM. In a preferred embodiment of the method for producing a liposomal formulation, the aqueous phase of step (ii) comprises a phosphate buffer at a concentration of about 10 mM and sodium chloride at a concentration of about 150 mM.

In some embodiments of the method for producing a liposomal formulation, the microfluidic device of step (ii) is a Y-junction, T-junction, or coaxial microfluidic device. In some embodiments of the method for producing a liposomal formulation, step (iii) and step (iv) comprise the use of tangential flow filtration (TFF). In some embodiments of the method for producing a liposomal formulation, step (iii) and step (iv) comprise the use of tangential flow filtration (TFF), wherein the TFF is diafiltration. In some embodiments of the method for producing a liposomal formulation, step (iii) and step (iv) comprise the use of tangential flow filtration (TFF), wherein the TFF is ultrafiltration. In some embodiments of the method for producing a liposomal formulation, step (iii) and step (iv) comprise the use of tangential flow filtration (TFF), wherein the TFF is diafiltration and ultrafiltration. In some embodiments of the method for producing a liposomal formulation, step (v) comprises the use of bioburden reduction filtration (BBR). In some embodiments of the method for producing a liposomal formulation, step (v) comprises the use of sterile filtration. In some embodiments of the method for producing a liposomal formulation, step (v) comprises the use of bioburden reduction filtration (BBR) and sterile filtration. In some embodiments of the method for producing a liposomal formulation, the saponin of step (vi) saponin is extracted from Quillaja Saponaria Molina. In some embodiments of the method for producing a liposomal formulation, the saponin of step (vi) is QS-21.

In some embodiments of the method for producing a liposomal formulation, the average size of the liposomes produced is between about 1 nanometer (nm) and about 1 μM in diameter. In some embodiments of the method for producing a liposomal formulation, the average size of the liposomes produced is about 1 nm, about 50 nm, about 100 nm, about 150 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, or about 1000 nm in diameter.

In some embodiments of the method for producing a liposomal formulation, the average size of the liposomes produced is between about 10 nanometer (nm) and about 500 nm in diameter. In some embodiments of the method for producing a liposomal formulation, the average size of the liposomes produced is between about 10 nanometer (nm) and about 200 nm in diameter. In some embodiments of the method for producing a liposomal formulation, the average size of the liposomes produced is between about 80 nanometer (nm) and about 200 nm in diameter. In some embodiments of the method for producing a liposomal formulation, the average size of the liposomes produced is between about 100 nanometer (nm) and about 170 nm in diameter. In some embodiments of the method for producing a liposomal formulation, the average size of the liposomes produced is about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, or about 170 nm in diameter.

In some embodiments of the method for producing a liposomal formulation, the polydispersity index (PDI) of the liposomes produced is less than about 0.5. In some embodiments of the method for producing a liposomal formulation, the PDI of the liposomes produced is about 0.1, about 0.2, about 0.3, about 0.4, or about 0.5. In some embodiments of the method for producing a liposomal formulation, the PDI of the liposomes produced is less than about 0.3. In some embodiments of the method for producing a liposomal formulation, the PDI of the liposomes produced is about 0.1, about 0.11, about 0.12, about 0. 13, about 0.14, about 0.15, about 0.16, about 0.17, about 0.18, about 0.19, about 0.2, about 0.21, about 0.22, about 0.23, about 0.24, about 0.25, about 0.26, about 0.27, about 0.28, about 0.29, or about 0.3.

### Linkers

In some embodiments, the TLR 7/8 modulating molecules disclosed herein comprise a linker that connects the TLR 7/8 modulating portion of the molecule to a terminal hydrophobic moiety. In some embodiments, the TLR 7/8 modulating molecules disclosed herein comprise a linker that connects the TLR 7/8 modulating portion of the molecule to a terminal hydrocarbon chain. In some embodiments, the TLR 7/8 modulating molecules disclosed herein comprise a linker that connects the TLR 7/8 modulating portion of the molecule to a terminal hydrophobic moiety, wherein the linker comprises polyethylene glycol (PEG). In some embodiments, the linker comprises one or more polyethylene glycol (PEG) repeat units. In some embodiments, the linker comprises PEG repeat units, wherein the number of PEG repeat units is an integer between about 1 and about 10. In some embodiments, the linker comprises PEG repeat units, wherein the number of PEG repeat units is an integer between about 1 and about 5. For example, in some embodiments, the linker comprises PEG repeat units, wherein the number of PEG repeat units is 1, 2, 3, 4, 5, 6, 7, 8, 9, or 10. In some embodiments, the linker comprises PEG repeat units, wherein the number of PEG repeat units is 1. In some embodiments, the linker comprises PEG repeat units, wherein the number of PEG repeat units is 2. In particular embodiments, the linker comprises PEG repeat units, wherein the number of PEG repeat units is 3. In other embodiments, the linker comprises PEG repeat units, wherein the number of PEG repeat units is 4. In other embodiments, the linker comprises PEG repeat units, wherein the number of PEG repeat units is 5.

In some embodiments, the TLR 7/8 modulating molecules disclosed herein comprise a linker that connects the TLR 7/8 modulating portion of the molecule to a terminal hydrophobic moiety, wherein the linker does not comprise PEG.

In various embodiments, the TLR 7/8 modulating molecules disclosed herein comprise a linker that connects the TLR 7/8 modulating portion of the molecule to a terminal hydrophobic moiety, wherein the linker comprises an alkyl group. For example, in some embodiments the linker comprises an alkyl group that contains 1, 2, 3, 4, 5, 6, 7, 8, 9, or 10 carbons. In a particular embodiment, the linker comprises an alkyl group that contains 2 carbons. In another particular embodiment, the linker comprises an alkyl group that contains 3 carbons. In another particular embodiment, the linker comprises an alkyl group that contains 4 carbons. In some embodiments, the TLR 7/8 modulating molecules disclosed herein comprise a linker that connects the TLR 7/8 modulating portion of the molecule to a terminal hydrophobic moiety, wherein the linker comprises a cyclic amine. In some embodiments, the TLR 7/8 modulating molecules disclosed herein comprise a linker that connects the TLR 7/8 modulating portion of the molecule to a terminal hydrophobic moiety, wherein the linker comprises a derivative of an amino acid.

### Pharmaceutical Compositions

In another embodiment, the disclosure comprises pharmaceutical compositions. For pharmaceutical composition purposes, the compound per se or pharmaceutically acceptable salt thereof will simply be referred to as the compounds of the disclosure.

A “pharmaceutical composition” refers to a mixture of one or more of the compounds of the disclosure, or a pharmaceutically acceptable salt, solvate, hydrate or prodrug thereof as an active ingredient, and at least one pharmaceutically acceptable excipient.

“Excipient” as used herein describes any ingredient other than the compound(s) of the disclosure. The choice of excipient will to a large extent depend on factors such as the mode of administration, the effect of the excipient on solubility and stability, and the nature of the dosage form.

As used herein, “excipient” includes any and all solvents, dispersion media, coatings, antibacterial and antifungal agents, isotonic and absorption delaying agents, carriers, diluents and the like that are physiologically compatible. Examples of excipients include one or more of water, saline, phosphate buffered saline, dextrose, glycerol, ethanol and the like, as well as combinations thereof, and may include isotonic agents, for example, sugar, sodium chloride, or polyalcohol such as mannitol, or sorbitol in the composition. Examples of excipients also include various organic solvents (such as hydrates and solvates). The pharmaceutical compositions may, if desired, contain additional excipients such as flavorings, binders/binding agents, lubricating agents, disintegrants, sweetening or flavoring agents, coloring matters or dyes, and the like. For example, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of excipients, therefore, also include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with additional excipients such as water, ethanol, propylene glycol, glycerin, or combinations thereof.

Examples of excipients also include pharmaceutically acceptable substances such as wetting agents or minor amounts of auxiliary substances such as wetting or emulsifying agents, preservatives, or buffers, which enhance the shelf life or effectiveness of the compound.

The compositions of this disclosure may be in a variety of forms. These include, for example, liquid, semi-solid and solid dosage forms, such as liquid solutions (e.g., injectable and infusible solutions), dispersions or suspensions, tablets, capsules, pills, powders, liposomes, lipid nanoparticles and suppositories. The form depends on the intended mode of administration and therapeutic application.

Typical compositions are in the form of injectable or infusible solutions, such as compositions similar to those used for passive immunization of humans with antibodies in general. One mode of administration is parenteral (e.g., intravenous, subcutaneous, intraperitoneal, intramuscular). In another embodiment, the compound is administered by intravenous infusion or injection. In yet another embodiment, the compound is administered by intramuscular or subcutaneous injection.

Oral administration of a solid dosage form may be, for example, presented in discrete units, such as hard or soft capsules, pills, cachets, lozenges, or tablets, each containing a predetermined amount of at least one compound of the disclosure. In another embodiment, the oral administration may be in a powder or granule form. In another embodiment, the oral dosage form is sub-lingual, such as, for example, a lozenge. In such solid dosage forms, the compounds of the disclosure are ordinarily combined with one or more adjuvants. Such capsules or tablets may comprise a controlled release formulation. In the case of capsules, tablets, and pills, the dosage forms also may comprise buffering agents or may be prepared with enteric coatings.

In another embodiment, oral administration may be in a liquid dosage form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as one or more of wetting, emulsifying, suspending, flavoring (e.g., sweetening), or perfuming agents.

In another embodiment, the disclosure comprises a parenteral dosage form. “Parenteral administration” includes, for example, subcutaneous injections, intravenous injections, intraperitoneally, intramuscular injections, intrasternal injections, and infusion. Injectable preparations (i.e., sterile injectable aqueous or oleaginous suspensions) may be formulated according to the known art using one or more of suitable dispersing, wetting agents, or suspending agents.

In another embodiment, the disclosure comprises a topical dosage form. “Topical administration” includes, for example, dermal and transdermal administration, such as via transdermal patches (with or without microneedle-mediated transdermal delivery) or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of this disclosure are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical excipients include alcohol, water, mineral oil, liquid petrolatum, white petrolatum, glycerin, polyethylene glycol and propylene glycol. Penetration enhancers may be incorporated-see, for example, B. C. Finnin and T. M. Morgan, J. Pharm. Sci., vol. 88, pp. 955-958, 1999.

Formulations suitable for topical administration to the eye include, for example, eye drops wherein the compound of this disclosure is dissolved or suspended in a suitable excipient. A typical formulation suitable for ocular or aural administration may be in the form of drops of a micronized suspension or solution in isotonic, pH-adjusted, sterile saline. Other formulations suitable for ocular and aural administration include ointments, biodegradable (i.e., absorbable gel sponges, collagen) and non-biodegradable (i.e., silicone) implants, wafers, lenses and particulate or vesicular systems, such as niosomes or liposomes. A polymer such as crossed linked polyacrylic acid, polyvinyl alcohol, hyaluronic acid, a cellulosic polymer, for example, hydroxypropylmethylcellulose, hydroxyethylcellulose, or methylcellulose, or a heteropolysaccharide polymer, for example, gelan gum, may be incorporated together with a preservative, such as benzalkonium chloride. Such formulations may also be delivered by iontophoresis.

For intranasal administration, the compounds of the disclosure are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1, 1,2-tetrafluoroethane or 1, 1,1,2,3,3,3-heptafluoropropane. For intranasal use, the powder may comprise a bioadhesive agent, for example, chitosan or cyclodextrin.

In another embodiment, the disclosure comprises a rectal dosage form. Such rectal dosage form may be in the form of, for example, a suppository. Cocoa butter is a traditional suppository base, but various alternatives may be used as appropriate.

Other excipients and modes of administration known in the pharmaceutical art may also be used. Pharmaceutical compositions of the disclosure may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; Rowe, Raymond C. Handbook of Pharmaceutical Excipients. Chicago, Pharmaceutical Press, 2005; Stahl, P. Heinrich and Camilli G. Wermuth, Eds. Handbook of Pharmaceutical Salts: Properties, Selection, and Use. New York: Wiley-VCH, 2011; and Brittain, Harry G., Ed. Polymorphism in Pharmaceutical Solids. New York: Informa Healthcare USA, Inc., 2016.

Acceptable excipients are nontoxic to subjects at the dosages and concentrations employed, and may comprise one or more of the following: 1) buffers such as phosphate, citrate, or other organic acids; 2) salts such as sodium chloride; 3) antioxidants such as ascorbic acid or methionine; 4) preservatives such as octadecyldimethylbenzyl ammonium chloride, hexamethonium chloride, benzalkonium chloride, benzethonium chloride, phenol, butyl or benzyl alcohol; 5)alkyl parabens such as methyl or propyl paraben, catechol, resorcinol, cyclohexanol, 3-pentanol, or m-cresol; 6) low molecular weight (less than about 10 residues) polypeptides; 7) proteins such as serum albumin, gelatin, or immunoglobulins; 8) hydrophilic polymers such as polyvinylpyrrolidone; 9) amino acids such as glycine, glutamine, asparagine, histidine, arginine, or lysine; 10) monosaccharides, disaccharides, or other carbohydrates including glucose, mannose, or dextrins; 11) chelating agents such as EDTA; 12) sugars such as sucrose, mannitol, trehalose or sorbitol; 13) salt-forming counter-ions such as sodium, metal complexes (e.g., Zn-protein complexes), or 14) non-ionic surfactants such as polysorbates (e.g., polysorbate 20 or polysorbate 80), poloxamers or polyethylene glycol (PEG).

Liposome containing compounds of the disclosure may be prepared by methods known in the art (See, for example, Chang, H. I.; Yeh, M. K.; Clinical development of liposome-based drugs: formulation, characterization, and therapeutic efficacy; Int J Nanomedicine 2012; 7; 49-60). Particularly useful liposomes may be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

Lipid nanoparticles (LNPs) comprising the compounds of the disclosure may be prepared by methods known in the art. Lipid nanoparticles (LNPs) constitute an alternative to other particulate systems, such as emulsions, liposomes, micelles, microparticles and/or polymeric nanoparticles, for the delivery of active ingredients, such as oligonucleotides, RNA and small molecule pharmaceuticals, and the adjuvant compounds of the disclosure. LNPs and their use for the delivery of oligonucleotides have been previously disclosed. See U.S. Pat. Nos. 7,691,405 and 11,406,706, U.S. Patent Application Publication Nos: US 2006/0083780, US 2006/0240554, US 2008/0020058, US 2009/0263407 and US 2009/0285881; and International Patent Application Publication Nos.: WO 2009/086558, WO2009/127060, WO2009/132131, WO2010/042877, WO2010/054384, WO2010/054401, WO2010/054405 and WO2010/054406. See also Semple et al., 2010, Nat. Biotechnol. 28:172-176. LNPs and their use for delivery of RNA vaccines have been previously disclosed. See International Patent Application Publication Nos.: WO2021213924 and WO2023019181. Lipid-based nanoparticles as pharmaceutical drug carriers have also been disclosed. See Puri et al., 2009, Crit. Rev. Ther. Drug Carrier Syst. 26:523-580. Cationic lipids are disclosed in U.S. Patent Application Publication Nos. US 2009/0263407, US 2009/0285881, US 2010/0055168, US 2010/0055169, US 2010/0063135, US 2010/0076055, US 2010/0099738 and US 2010/0104629, and U.S. Pat. No. 10,166,298. Lipid nanoparticle capsules are described in U.S. Patent Application Publication No. 2013/0017239. The compounds of the disclosure may be embedded in the lipid layer of the LNP for targeting of the LNP comprising an active ingredient (i.e. oligonucleotide, RNA, small molecule, etc) to the lymph nodes via the TLR7/8 modulating moiety of the compounds of the disclosure.

Compounds of the disclosure may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, lipid nanoparticles, albumin microspheres, microemulsions, nanoparticles and nanocapsules) or in macroemulsions. Such techniques are disclosed in Remington, The Science and Practice of Pharmacy, 20th Ed., Mack Publishing (2000).

Sustained-release preparations may be used. Suitable examples of sustained-release preparations include semi-permeable matrices of solid hydrophobic polymers containing a compound of the disclosure, which matrices are in the form of shaped articles, e.g., films, or microcapsules. Examples of sustained-release matrices include polyesters, hydrogels (for example, poly(2-hydroxyethyl-methacrylate), or ‘poly (vinylalcohol)), polylactides, copolymers of L-glutamic acid and 7 ethyl-L-glutamate, non-degradable ethylene-vinyl acetate, degradable lactic acid-glycolic acid copolymers such as those used in leuprolide acetate for depot suspension (injectable microspheres composed of lactic acid-glycolic acid copolymer and leuprolide acetate), sucrose acetate isobutyrate, and poly-D-(−)-3-hydroxybutyric acid.

The formulations to be used for intravenous administration must be sterile. This is readily accomplished by, for example, filtration through sterile filtration membranes. Compounds of the disclosure are generally placed into a container having a sterile access port, for example, an intravenous solution bag or vial having a stopper pierceable by a hypodermic injection needle.

Suitable emulsions may be prepared using commercially available fat emulsions, such as a lipid emulsions comprising soybean oil, a fat emulsion for intravenous administration (e.g., comprising safflower oil, soybean oil, egg phosphatides and glycerin in water), emulsions containing soya bean oil and medium-chain triglycerides, and lipid emulsions of cottonseed oil. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion may comprise fat droplets between 0.1 and 1.0 μm, particularly 0.1 and 0.5 μm, and have a pH in the range of 5.5 to 8.0.

For example, the emulsion compositions may be those prepared by mixing a compound of the disclosure with a lipid emulsions comprising soybean oil or the components thereof (soybean oil, egg phospholipids, glycerol and water).

Compositions for inhalation or insufflation include solutions and suspensions in pharmaceutically acceptable aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

A drug product intermediate (DPI) is a partly processed material that must undergo further processing steps before it becomes bulk drug product. Compounds of the disclosure may be formulated into drug product intermediate DPI containing the active ingredient in a higher free energy form than the crystalline form. One reason to use a DPI is to improve oral absorption characteristics due to low solubility, slow dissolution, improved mass transport through the mucus layer adjacent to the epithelial cells, and in some cases, limitations due to biological barriers such as metabolism and transporters. Other reasons may include improved solid state stability and downstream manufacturability. In one embodiment, the drug product intermediate contains a compound of the disclosure isolated and stabilized in the amorphous state (for example, amorphous solid dispersions (ASDs)). There are many techniques known in the art to manufacture ASD's that produce material suitable for integration into a bulk drug product, for example, spray dried dispersions (SDD's), melt extrudates (often referred to as HME's), co-precipitates, amorphous drug nanoparticles, and nano-adsorbates. In one embodiment amorphous solid dispersions comprise a compound of the disclosure and a polymer excipient. Other excipients as well as concentrations of said excipients and the compound of the disclosure are well known in the art and are described in standard textbooks. See, for example, “Amorphous Solid Dispersions Theory and Practice” by Navnit Shah et al.

### Administration and Dosing

The term “treating”, “treat” or “treatment” as used herein embraces both preventative, i.e., prophylactic, and palliative treatment, i.e., relieve, alleviate, or slow the progression of the patient's disease (or condition) or any tissue damage associated with the disease.

As used herein, the terms, “subject, “individual” or “patient,” used interchangeably, refer to any animal, including mammals. Mammals according to the disclosure include canine, feline, bovine, caprine, equine, ovine, porcine, rodents, lagomorphs, primates, humans and the like, and encompass mammals in utero. In an embodiment, humans are suitable subjects. Human subjects may be of any gender and at any stage of development.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which may include one or more of the following:
(1) preventing the disease; for example, preventing a disease, condition or disorder in an individual that may be predisposed to the disease, condition or disorder but does not yet experience or display the pathology or symptomatology of the disease;
(2) inhibiting the disease; for example, inhibiting a disease, condition or disorder in an individual that is experiencing or displaying the pathology or symptomatology of the disease, condition or disorder (i.e., arresting (or slowing) further development of the pathology or symptomatology or both); and
(3) ameliorating the disease; for example, ameliorating a disease, condition or disorder in an individual that is experiencing or displaying the pathology or symptomatology of the disease, condition or disorder (i.e., reversing the pathology or symptomatology or both).

Typically, a compound of the disclosure is administered in an amount effective to treat a condition as described herein. The compounds of the disclosure may be administered as compound per se, or alternatively, as a pharmaceutically acceptable salt. For administration and dosing purposes, the compound per se or pharmaceutically acceptable salt thereof will simply be referred to as the compounds of the disclosure.

The compounds of the disclosure are administered by any suitable route in the form of a pharmaceutical composition adapted to such a route, and in a dose effective for the treatment intended. The compounds of the disclosure may be administered orally, rectally, vaginally, parenterally, topically, intranasally, or by inhalation.

The compounds of the disclosure may be administered orally. Oral administration may involve swallowing, so that the compound enters the gastrointestinal tract, or buccal or sublingual administration may be employed by which the compound enters the bloodstream directly from the mouth.

In another embodiment, the compounds of the disclosure may also be administered parenterally, for example directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors, and infusion techniques.

In another embodiment, the compounds of the disclosure may also be administered topically to the skin or mucosa, that is, dermally or transdermally. In another embodiment, the compounds of the disclosure may also be administered intranasally or by inhalation. In another embodiment, the compounds of the disclosure may be administered rectally or vaginally. In another embodiment, the compounds of the disclosure may also be administered directly to the eye or ear.

The dosage regimen for the compounds of the disclosure or compositions containing said compounds is based on a variety of factors, including the type, age, weight, sex and medical condition of the patient; the severity of the condition; the route of administration; and the activity of the particular compound employed. Thus, the dosage regimen may vary widely. In one embodiment, the total daily dose of a compound of the disclosure is typically from about 0.01 to about 100 mg/kg (i.e., mg compound of the disclosure per kg body weight) for the treatment of the indicated conditions discussed herein. In another embodiment, total daily dose of the compound of the disclosure is from about 0.1 to about 50 mg/kg, and in another embodiment, from about 0.5 to about 30 mg/kg. It is not uncommon that the administration of the compounds of the disclosure will be repeated a plurality of times in a day (typically no greater than 4 times). Multiple doses per day typically may be used to increase the total daily dose, if desired.

### Therapeutic Methods and Uses

The compounds of the disclosure may agonize or modulate the activity of TLR7 and/or TLR8 and may be useful as vaccine adjuvants.

Adjuvant formulations comprising the compounds of the disclosure may be used with an immunogen (i.e. a therapeutic agent or antigen of interest) to obtain an immunogenic composition, for example, a vaccine. The immunogenic composition may comprise naturally-occurring or artificially-created proteins, recombinant proteins, glycoproteins, peptides, carbohydrates, nucleic acids, haptens, whole viruses, bacteria, protozoa, or virus-like particles, or conjugates thereof as the immunogen. The immunogenic composition may be suitably used as a vaccine for chickenpox or shingles, human respiratory syncytial virus infection (RSV), Cytomegalovirus infection (CMV), Human metapneumovirus, Human parainfluenza viruses type 1 or type 3, Lyme disease, Streptococcus pneumonia, Clostridioides difficile, Escherichia coli or Klebsiella pneumoniae, influenza, HIV-1, Hepatitis A, Hepatitis B, Human Papilloma virus, Meningococcal type A meningitis, Meningococcal type B meningitis, Meningococcal type C meningitis, Tetanus, Diphtheria, Pertussis, Polio, Haemophilus influenza type B, Dengue, Hand Foot and Mouth Disease, Typhoid, Pneumococcus, Japanese encephalitis virus, Anthrax, Shingles, Malaria, Norovirus, or cancer. The immunogenic composition may be suitably used in methods for treating or preventing a disease or infection in a subject, preferably wherein the subject is a human, caused by a pathogen associated with an infectious disease wherein the pathogen is selected from Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, CJD prion, Clonorchis sinensis, Clostridium botulinum, Clostridium difficile, Clostridium perfringens, Clostridium perfringens, Clostridium spp, Clostridium tetani, Coccidioides spp, coronaviruses, Corynebacterium diphtheriae, Coxiella burnetii, Crimean-Congo hemorrhagic fever virus, Cryptococcus neoformans, Cryptosporidium genus, Cytomegalovirus (CMV), Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli, Fasciola hepatica and Fasciola gigantica, FFI prion, Filarioidea superfamily, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBOV), Human herpesvirus 6 (HHV-6) and Human herpesvirus 7 (HHV-7), Human metapneumovirus (hMPV) including hMPV A and hMPV B, hMPV F protein, Human papillomavirus (HPV), Human parainfluenza viruses (HPIV), Japanese encephalitis virus, JC virus, Junin virus, Kingella kingae, Klebsiella granulomatis, Klebsiella pneumoniae, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowleri, Necator americanus, Neisseria gonorrhoeae, Neisseria meningitidis, Nocardia asteroides, Nocardia spp, Onchocerca volvulus, Orientia tsutsugamushi, Orthomyxoviridae family (Influenza), Paracoccidioides brasiliensis, Paragonimus spp, Paragonimus westermani, Parainfluenza virus (PIV) including PIV1, PIV2, PIV3, and PIV4, PIV1 F protein, PIV3 F protein, Parvovirus B19, Pasteurella genus, Plasmodium genus, Pneumocystis jirovecii, Poliovirus, Rabies virus, Respiratory syncytial virus (RSV), Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, Schistosoma genus, Shigella genus, Sin Nombre virus, Hantavirus, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella-zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, and Yersinia pseudotuberculosis.

In some embodiments, adjuvant formulations comprising the compounds disclosed herein may be used with a polypeptide immunogen to obtain an immunogenic composition. In some embodiments, adjuvant formulations comprising the compounds disclosed herein may be used with more than one polypeptide immunogen to obtain an immunogenic composition, for example, 2, 3, 4, 5, or more polypeptide immunogens.

In some embodiments, adjuvant formulations comprising the compounds disclosed herein may be used with an immunogen to obtain an immunogenic composition, wherein the immunogen is a nucleic acid encoding a polypeptide. In various embodiments, adjuvant formulations comprising the compounds disclosed herein may be used with an immunogen to obtain an immunogenic composition, wherein the immunogen is DNA encoding a polypeptide. In a particular embodiment, adjuvant formulations comprising the compounds disclosed herein may be used with an immunogen to obtain an immunogenic composition, wherein the immunogen is RNA encoding a polypeptide. In some embodiments, adjuvant formulations comprising the compounds disclosed herein may be used with more than one immunogen to obtain an immunogenic composition, wherein at least one immunogen is a nucleic acid encoding a polypeptide. In other embodiments, adjuvant formulations comprising the compounds disclosed herein may be used with more than one immunogen to obtain an immunogenic composition, wherein at least one immunogen is DNA encoding a polypeptide. In a particular, adjuvant formulations comprising the compounds disclosed herein may be used with more than one immunogen to obtain an immunogenic composition, wherein at least one immunogen is RNA encoding a polypeptide.

In some embodiments, adjuvant formulations comprising the compounds disclosed herein may be used with a Streptococcus pneumoniae antigen to obtain an immunogenic composition. In other embodiments, adjuvant formulations comprising the compounds disclosed herein may be used with a Streptococcus pneumoniae polysaccharide antigen to obtain an immunogenic composition. In other embodiments, adjuvant formulations comprising the compounds disclosed herein may be used with a Streptococcus pneumoniae serotype 3 polysaccharide antigen to obtain an immunogenic composition. In some embodiments, adjuvant formulations comprising the compounds disclosed herein may be used with 1, 2, 3, 4, 5, 6, 7, 8, 9, 10, 11, 12, 13, 14, 15, 16, 17, 18, 19, 20, 21, 22, 23, 24, 25, or more Streptococcus pneumoniae polysaccharide antigens.

In some embodiments, adjuvant formulations comprising the compounds disclosed herein may be used with a conjugated Streptococcus pneumoniae antigen to obtain an immunogenic composition. In other embodiments, adjuvant formulations comprising the compounds disclosed herein may be used with a conjugated Streptococcus pneumoniae polysaccharide antigen to obtain an immunogenic composition. In other embodiments, adjuvant formulations comprising the compounds disclosed herein may be used with a conjugated Streptococcus pneumoniae serotype 3 polysaccharide antigen to obtain an immunogenic composition. In some embodiments, adjuvant formulations comprising the compounds disclosed herein may be used with 1, 2, 3, 4, 5, 6, 7, 8, 9, 10, 11, 12, 13, 14, 15, 16, 17, 18, 19, 20, 21, 22, 23, 24, 25, or more conjugated Streptococcus pneumoniae polysaccharide antigens. In some embodiments, the Streptococcus pneumoniae polysaccharide antigens are conjugated to a CRM197 carrier. In some embodiments, the Streptococcus pneumoniae polysaccharide antigens are conjugated to a C5a peptidase from Streptococcus (SCP) carrier. In a particular embodiment, the immunogenic composition comprises an adjuvant compound disclosed herein and a Streptococcus pneumoniae serotype 3 polysaccharide antigen conjugated to a CRM197 carrier. In another particular embodiment, the immunogenic composition comprises an adjuvant compound disclosed herein and a Streptococcus pneumoniae serotype 3 polysaccharide antigen conjugated to a SCP carrier.

The present disclosure provides an immunogenic composition comprising an immunogen and a compound of the disclosure as described herein.

### Co-Administration

The compounds of the disclosure may be used alone, or in combination with one or more therapeutic agents. The disclosure provides any of the uses, methods or compositions as defined herein wherein the compound of the disclosure, or pharmaceutically acceptable salt thereof, is used in combination with one or more therapeutic agent discussed herein.

The administration of two or more compounds “in combination” means that all of the compounds are administered closely enough in time to affect treatment of the subject. The two or more compounds may be administered simultaneously or sequentially, via the same or different routes of administration, on same or different administration schedules and with or without specific time limits depending on the treatment regimen. Additionally, simultaneous administration may be carried out by mixing the compounds prior to administration or by administering the compounds at the same point in time but as separate dosage forms at the same or different site of administration. Examples of “in combination” include, but are not limited to, “concurrent administration,” “co-administration,” “simultaneous administration,” “sequential administration” and “administered simultaneously”.

A compound of the disclosure and the one or more therapeutic agents may be administered as a fixed or non-fixed combination of the active ingredients. The term “fixed combination” means a compound of the disclosure, or a pharmaceutically acceptable salt thereof, and the one or more therapeutic agents, are both administered to a subject simultaneously in a single composition or dosage. The term “non-fixed combination” means that a compound of the disclosure, or a pharmaceutically acceptable salt thereof, and the one or more therapeutic agents are formulated as separate compositions or dosages such that they may be administered to a subject in need thereof simultaneously or at different times with variable intervening time limits, wherein such administration provides effective levels of the two or more compounds in the body of the subject.

In one embodiment, the present disclosure provides a pharmaceutical composition comprising an immunogen disclosed herein, wherein the pharmaceutical composition is administered in combination with a pharmaceutical composition comprising an adjuvant disclosed herein or a pharmaceutically acceptable salt thereof simultaneously or at different times.

These agents and compounds of the disclosure may be combined with pharmaceutically acceptable vehicles such as saline, Ringer's solution, dextrose solution, and the like. The particular dosage regimen, i.e., dose, timing and repetition, will depend on the particular individual and that individual's medical history.

### Kits

Another aspect of the disclosure provides kits comprising the compound of the disclosure or pharmaceutical compositions comprising the compound of the disclosure. A kit may include, in addition to the compound of the disclosure or pharmaceutical composition thereof, diagnostic or therapeutic agents. A kit may also include instructions for use in a diagnostic or therapeutic method. In some embodiments, the kit includes the compound or a pharmaceutical composition thereof and a diagnostic agent. In other embodiments, the kit includes the compound or a pharmaceutical composition thereof and one or more therapeutic agents, such as an immunogen disclosed herein.

In yet another embodiment, the disclosure comprises kits that are suitable for use in performing the methods of treatment described herein. In one embodiment, the kit contains a first dosage form comprising one or more of the compounds of the disclosure in quantities sufficient to carry out the methods of the disclosure. In another embodiment, the kit comprises one or more compounds of the disclosure in quantities sufficient to carry out the methods of the disclosure and a container for the dosage.

### Synthetic Methods

Compounds of the present disclosure may be synthesized by synthetic routes that include processes analogous to those well-known in the chemical arts, particularly in light of the description contained herein. The starting materials are generally available from commercial sources or may be prepared using methods well known to those skilled in the art. Many of the compounds used herein, are related to, or may be derived from compounds in which one or more of the scientific interest or commercial need has occurred. Accordingly, such compounds may be one or more of 1) commercially available; 2) reported in the literature or 3) prepared from other commonly available substances by one skilled in the art using materials which have been reported in the literature.

For illustrative purposes, the reaction schemes depicted below provide potential routes for synthesizing the compounds of the present disclosure as well as key intermediates. For a more detailed description of the individual reaction steps, see the Examples section below. Those skilled in the art will appreciate that other synthetic routes may be used to synthesize the inventive compounds. Although specific starting materials and reagents are discussed below, other starting materials and reagents may be substituted to provide one or more of a variety of derivatives or reaction conditions. In addition, many of the compounds prepared by the methods described below may be further modified in light of this disclosure using conventional chemistry well known to those skilled in the art.

The skilled person will appreciate that the experimental conditions set forth in the schemes that follow are illustrative of suitable conditions for effecting the transformations shown, and that it may be necessary or desirable to vary the precise conditions employed for the preparation of compounds of the disclosure. It will be further appreciated that it may be necessary or desirable to carry out the transformations in a different order from that described in the schemes, or to modify one or more of the transformations, to provide the desired compound of the disclosure.

In the preparation of compounds of the disclosure it is noted that some of the preparation methods useful for the preparation of the compounds described herein may require protection of remote functionality (e.g., a primary amine, secondary amine, carboxyl, etc. in a precursor of a compound of the disclosure). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see March's Advanced Organic Chemistry: Reactions, Mechanisms, and Structure 8th Edition.

For example, if a compound contains a amine or carboxylic acid functionality, such functionality may interfere with reactions at other sites of the molecule if left unprotected. Accordingly, such functionalities may be protected by an appropriate protecting group (PG) which may be removed in a subsequent step. Suitable protecting groups for amine and carboxylic acid protection include those protecting groups commonly used in peptide synthesis (such as N-t-butoxycarbonyl (Boc), benzyloxycarbonyl (Cbz), and 9-fluorenylmethylenoxycarbonyl (Fmoc) for amines and lower alkyl or benzyl esters for carboxylic acids) which are generally not chemically reactive under the reaction conditions described and may typically be removed without chemically altering other functionality in a compound of the disclosure.

### General Experimental Details

In the non-limiting Examples and Preparations that illustrate the disclosure and that are set out in the description, and in the following Schemes, the following the abbreviations, definitions and analytical procedures may be referred to.

The compounds and intermediates described below were named using the naming convention provided with ChemDraw version 20.1.1.123. The naming convention provided with ChemDraw version 20.1.1.123 is well known by those skilled in the art and it is believed that the naming convention generally comports with the IUPAC (International Union for Pure and Applied Chemistry) recommendations on Nomenclature of Organic Chemistry and the CAS Index rules. Unless noted otherwise, all reactants were obtained commercially without further purifications or were prepared using methods known in the literature.

The following illustrate the synthesis of various compounds of the present disclosure. Additional compounds within the scope of this disclosure may be prepared using the methods illustrated in these Examples, either alone or in combination with techniques generally known in the art. All starting materials in these Preparations and Examples are either commercially available or can be prepared by methods known in the art or as described herein.

Reactions were performed in air or, when oxygen- or moisture-sensitive reagents or intermediates were employed, under an inert atmosphere (nitrogen or argon).

Unless otherwise noted, chemical reactions were performed at room temperature (about 25° C.).

In the examples, proton nuclear magnetic resonance (¹H NMR) spectra were recorded at 400 MHz, where δ is chemical shift; br is broad; CDCl₃ is deuterated chloroform; (CD₃)₂SO is deuterated dimethyl sulfoxide; d is doublet; dd is doublet of doublets; ddd is doublet of doublet of doublets; D₂O is deuterated water; dt is doublet of triplets; s is singlet; t is triplet; m is multiplet; MHz is megahertz; ppm is parts per million; q is quartet.

### Abbreviations

Å is angstrom;
AcOH is acetic acid;
MeCN is acetonitrile;
NH₄OH is ammonium hydroxide;
Boc2O is di-tert-butyl decarbonate;
9-BBN is 9-borabicyclo[3.3.1]nonane;
CHCl3 is chloroform;
° C. is degrees Celsius;
Cs2CO3 is cesium carbonate;
CuI is copper (I) iodide;
DCM is dichloromethane;
DIH is 1,3-diiodo-5,5-dimethylhydantoin;
DIPEA is N, N-diisopropylethylamine;
DMA is dimethylacetamide;
DMF is dimethylformamide;
DMP is Dess-Martin periodinane;
EtOAc is ethyl acetate;
EtOH is ethanol;
g is gram;
H2 is hydrogen;
HATU is 1-[bis(dimethylamino)methylene]-1H-1,2,3-triazolo[4,5-b]pyridinium 3-oxid
hexafluorophosphate;
HCl is hydrochloric acid;
H2O is water;
IPA is isopropyl alcohol;
KOAc is potassium acetate;
K2CO3 is potassium carbonate;
KOH is potassium hydroxide;
LCMS is liquid chromatography mass spectrometry;
LiOH is lithium hydroxide monohydrate;
L is liter;
M is molar;
M+ is mass ion;
mm is millimeter;
m-CPBA is 3-chloroperoxybenzoic acid;
MeOH is methanol;
μm is micromole;
mL is milliliter;
mL/min is milliliter per minute;
mmol is millimole;
mol is mole;
MTBE is methyl tert-butyl ether;
psi is pounds per square inch;
N2 is nitrogen;
nm is nanometer;
Rf is retention factor;
NaBH3CN is sodium cyanoborohydride;
NaHSO3 is sodium bisulfite;
NaHCO3 is sodium bicarbonate;
Na2CO3 is sodium carbonate;
NaOH is sodium hydroxide;
Na2SO3 is sodium sulfite;
Na2SO4 is sodium sulfate;
Pd/C is palladium on carbon;
Pd (PPh3) 4 is tetrakis (triphenylphosphine) palladium (0);
Pt/C is platinum on carbon;
TBSCl is tert-butyldimethylsilyl chloride;
TEA is triethylamine;
THF is tetrahydrofuran;
TFA is trifluoroacetic acid;
TsCl is p-toluenesulfonyl chloride;
UV is ultraviolet; and
wt % is weight percent.

### General Methods

Unless stated otherwise, the variables in Preparation P1-P8 have the same meanings as defined herein.

In some cases, compounds described herein may contain protecting groups, which may be appended or removed by additional steps in the synthetic sequence using conditions known in the art (March's Advanced Organic Chemistry: Reactions, Mechanisms, and Structure 8th Edition or Protecting Groups, 10 Georg Thieme Verlag, 1994; Wuts, Greene's Protective Groups in Organic Synthesis (Fifth edition/2014)). Compounds at every step may be purified by standard techniques, such as column chromatography, crystallization, or reverse phase SFC or HPLC. The following substrates were synthesized according to Preparation P1-P8.

### Preparation P1

### 2-((4-Amino-2-butyl-1H-imidazo[4,5-c]quinolin-1-yl)methyl)-2-(aminomethyl) propane-1,3-diol hydrochloride (P1)

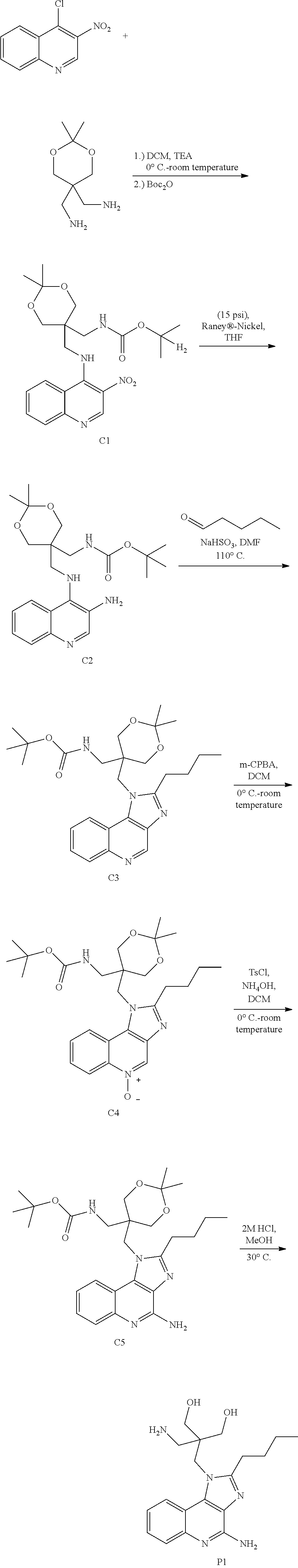

Step 1. Preparation of tert-butyl ((2,2-dimethyl-5-(((3-nitroquinolin-4-yl)amino)methyl)-1,3-dioxan-5-yl)methyl)carbamate (C1)

To a solution of (2,2-dimethyl-1,3-dioxane-5,5-diyl)dimethanamine (CAS: 104275 Oct. 7; 84.0 g, 482 mmol) in DCM (2.5 L) was added TEA (97.6 g, 964 mmol) and 4-chloro-3-nitroquinoline (CAS: 39061-97-7; 50.3 g, 241 mmol) at 0° C. The reaction mixture was warmed to room temperature and stirred for 3.5 hours before Boc2O (CAS: 24424-99-5; 237 g, 1.09 mol) was added. The reaction mixture was stirred at room temperature for an additional 16 hours then was washed with the brine (1 L). The organic layer was concentrated in vacuo then MeCN (150 mL) was added, and the reaction mixture was filtered. The filter cake was washed with MeCN (100 mL) then MTBE (200 mL). The filter cake dried further to provide C1 (222 g, >99% yield) as a yellow solid. The solid was used directly in the next step without additional purification.

Step 2. Preparation of tert-butyl ((5-(((3-aminoquinolin-4-yl)amino)methyl)-2,2-dimethyl-1,3-dioxan-5-yl)methyl)carbamate (C2)

The following reaction was conducted in 4 batches in parallel then combined for work-up. For the first batch, Raney®-Nickel (35.0 g) was added to a solution of C1 (70.0 g, 157 mmol) in THF (1 L) then was stirred under H₂ gas (15 psi) at room temperature for 16 hours.

The 3 additional batches of the same reaction were all conducted with C1 (70.0 g, 157 mmol). All 4 batches were combined then the combined reaction mixture was filtered. The filter cake was washed with DCM (300 mL×3). The combined filtrate was concentrated in vacuo to provide C2 (217 g, 48.2% yield) as a brown solid. The solid was used directly in the next step without additional purification. (LCMS)(M+H)⁺ 417.3.

Step 3. Preparation of tert-butyl ((5-((2-butyl-1H-imidazo[4,5-c]quinolin-1-yl)methyl)-2,2-dimethyl-1,3-dioxan-5-yl)methyl)carbamate (C3)

To the reaction mixture of pentanal (44.9 g, 521 mmol) and NaHSO3 (81.3 g, 781 mmol) were added to a solution of C2 (217 g, 521 mmol) in DMF (2.5 L). The reaction mixture was stirred at 110° C. for 16 hours then diluted with H₂O (3 L) and filtered. The filter cake of the combined batches was diluted with EtOAc (1 L) then heated to 80° C. until the solid dissolved. The solution was cooled to room temperature and stirred for 2 hours which caused an off-white solid to form. The reaction mixture was filtered, and the filter cake was collected to provide C3 (74.5 g, 29.6% yield) as a white solid. The solid was used directly in the next step without additional purification. (LCMS)(M+H)⁺ 483.4. ¹H NMR (400 MHz, CDCl₃) δ 9.30 (s, 1H), 8.34 (d, 1H), 8.28 (dd, 1H), 7.67 (ddd, 1H), 7.60 (ddd, 1H), 5.07-4.90 (m, 1H), 4.78-4.68 (m, 1H), 4.65-4.48 (m, 1H), 3.90-3.65 (m, 3H), 3.39 (d, 3H), 3.10-2.87 (m, 2H), 1.97-1.87 (m, 2H), 1.54-1.43 (m, 11H), 1.31 (br s, 3H), 1.09 (br s, 3H), 1.00 (t, 3H).

Step 4. Preparation of 1-((5-(((tert-butoxycarbonyl)amino)methyl)-2,2-dimethyl-1,3-dioxan-5-yl)methyl)-2-butyl-1H-imidazo[4,5-c]quinoline 5-oxide (C4)

Under N₂ gas at 0-10° C., m-CPBA (23.3 g, 115 mmol) was added to a solution of C3 (37.0 g, 76.7 mmol) in DCM (320 mL). The reaction mixture was stirred at room temperature for 48 hours then quenched with saturated Na₂SO₃ (80 mL). The quenched reaction mixture was washed with saturated NaHCO₃ (100 mL×4) then brine (100 mL) and dried with Na₂SO₄. The reaction mixture was filtered and concentrated in vacuo to provide C4 (50.0 g, >99% yield) as a yellow solid. The solid was used directly in the next step without additional purification. (LCMS) (M+H)⁺ 499.3.

Step 5. Preparation of tert-butyl ((5-((4-amino-2-butyl-1H-imidazo[4,5-c]quinolin-1-yl)methyl)-2.2-dimethyl-1,3-dioxan-5-yl)methyl)carbamate (C5)

At 0° C., to a solution of C4 (50.0 g, 0.100 mol) in DCM (500 mL) was added NH₄OH solution (120 mL) then TsCl (23.9 g, 125 mmol). The reaction mixture was warmed to room temperature then stirred for 16 hours. The yellow reaction mixture was washed with saturated NaHCO₃ (100 mL×2) then brine (100 mL×2) and dried with Na₂SO₄. The reaction mixture was filtered then concentrated in vacuo to give a brown residue. The residue was purified by column chromatography (silica gel, MeOH: DCM, 1-10% MeOH over 15 minutes) then lyophilized to provide C5 (31.9 g, 69.3% yield) as a light-yellow solid. (LCMS)(M+H)⁺ 498.3. ¹H NMR (400 MHz, (CD₃)₂SO) δ 8.29 (d, 1H), 7.60 (dd, 1H), 7.40 (t, 1H), 7.23-7.13 (m, 2H), 6.52 (br s, 2H), 4.85 (d, 1H), 4.64 (d, 1H), 3.89-3.62 (m, 3H), 3.22-2.76 (m, 4H), 1.84-1.71 (m, 2H), 1.46-1.34 (m, 11H), 1.26 (br s, 4H), 1.08 (br s, 3H), 0.94 (t, 3H).

Step 6. Preparation of 2-((4-amino-2-butyl-1H-imidazo[4,5-c]quinolin-1-yl)methyl)-2-(aminomethyl) propane-1,3-diol hydrochloride (P1)

At 0-5° C., to a solution of C5 (31.9 g, 64.1 mmol) in MeOH (50 mL) was added 2M HCl in MeOH (600 mL). The reaction mixture was heated to 30° C. and stirred for 2 hours then was concentrated in vacuo. The residue was suspended in EtOAc/DCM/MTBE then filtered. The filter cake was collected then lyophilized to provide P1 (27.3 g, >99% yield) as a white solid. The solid was used directly in the next step without further purification. (LCMS)(M+H)⁺ 358.2. ¹H NMR (400 MHz, (CD₃)₂SO) δ 14.16-13.94 (m, 1H), 8.98 (br s, 1H), 8.67 (d, 1H), 7.98 (br s, 2H), 7.81 (d, 1H), 7.69 (t, 1H), 7.52 (t, 1H), 4.99 (d, 1H), 4.67 (d, 1H), 4.10 (br s, 2H), 3.61 (dd, 2H), 3.41 (d, 1H), 3.30 (d, 1H), 3.20-2.95 (m, 3H), 2.70-2.59 (m, 1H), 1.87-1.74 (m, 2H), 1.49-1.36 (m, 2H) 0.94 (t, 3H).

### Preparation P2

### 1-(3-Amino-2,2-bis(((tert-butyldimethylsilyl)oxy)methyl)propyl)-2-butyl-1H-imidazo[4,5-c]quinolin-4-amine (P2)

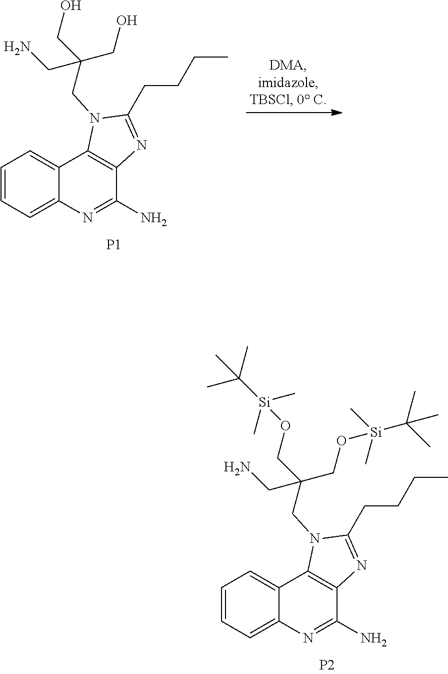 [SMILES: CCCCc1nc2c(N)nc3ccccc3c2n1CC(CN)(CO)CO ; CCCCc1nc2c(N)nc3ccccc3c2n1CC(CN)(CO[Si](C)(C)C(C)(C)C)CO[Si](C)(C)C(C)(C)C]

Preparation of 1-(3-amino-2,2-bis(((tert-butyldimethylsilyl)oxy)methyl)propyl)-2-butyl-1H-imidazo[4,5-c]quinolin-4-amine (P2)

At 0° C. under N₂ gas, to a solution of P1 (29.3 g, 68.1 mmol) in DMA (250 mL) was added imidazole (32.4 g, 476 mmol) and TBSCl (51.3 g, 340 mmol). The reaction mixture was stirred for 2 hours then added dropwise to ice water (600 mL). The diluted reaction mixture was extracted with DCM (300 mL×3) then the combined organic layer was washed with NaHCO₃ (100 mL×4), brine (100 mL×2) and dried with Na₂SO₄. The dried organic layer was filtered and concentrated in vacuo. The yellow gum was purified by column chromatography (silica gel, ((1:10) NH₄OH: MeOH) in DCM, 0-15% ((1:10) NH₄OH in MeOH)) to provide P2 (30.6 g, 76.7% yield) as a light-yellow solid. (LCMS)(M+H)⁺ 587.9. ¹H NMR (400 MHz, (CD₃)₂SO) δ 8.53 (d, 1H), 7.59 (d, 1H), 7.38 (t, 1H), 7.14 (t, 1H), 6.39 (br s, 2H), 4.94 (d, 1H), 4.52 (d, 1H), 3.76-3.63 (m, 2H), 3.54-3.45 (m, 1H), 3.41-3.36 (m, 1H), 3.13-2.70 (m, 3H), 2.34-2.22 (m, 1H), 1.84-1.57 (m, 4H), 1.40 (q, 2H), 0.92 (t, 3H), 0.84 (br s, 18H), 0.020-(−0.13)(t, 12H).

### Preparation P3

### Tert-butyl 4-(3-(3-borabicyclo[3.3.1]nonan-3-yl)propyl) piperazine-1-carboxylate (P3)

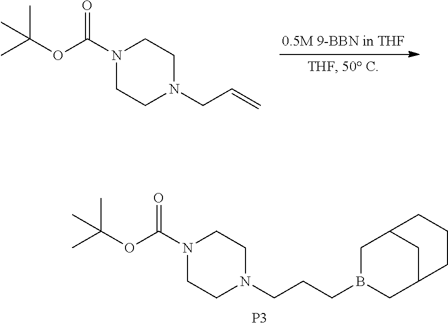 [SMILES: C=CCN1CCN(C(=O)OC(C)(C)C)CC1 ; CC(C)(C)OC(=O)N1CCN(CCCB2CC3CCCC(C2)C3)CC1]

Preparation of tert-butyl 4-(3-(3-borabicyclo[3.3.1]nonan-3-yl)propyl) piperazine-1-carboxylate (P3)

The following reaction was conducted in 7 batches in parallel then combined for work-up. To a solution of 1, 1-dimethylethyl 4-(2-propen-1-yl)-1-piperazinecarboxylate (CAS: 77278-75-2; 10.0 g, 44.0 mmol) in THF (60 mL) was added 0.5M 9-BBN in THF (88.0 mL, 44.0 mmol) to prepare the first batch. The reaction mixture of the first batch was degassed with N₂ gas then stirred at 50° C. for 30 minutes.

The additional 6 batches of the same reaction were conducted with 1.1-dimethylethyl 4-(2-propen-1-yl)-1-piperazinecarboxylate (CAS: 77278-75-2; 10.0 g, 44.0 mmol) each. The 7 batches were combined to form P3 (115 g, 96.2% yield) as a clear crude solution. The clear crude solution was used directly in the next step without further purification.

### Preparation P4

### 2-((4-Amino-2-butyl-7-(3-(piperazin-1-yl)propyl)-1H-imidazo[4,5-c]quinolin-1-yl)methyl)-2-methylpropane-1,3-diol hydrochloride (P4)

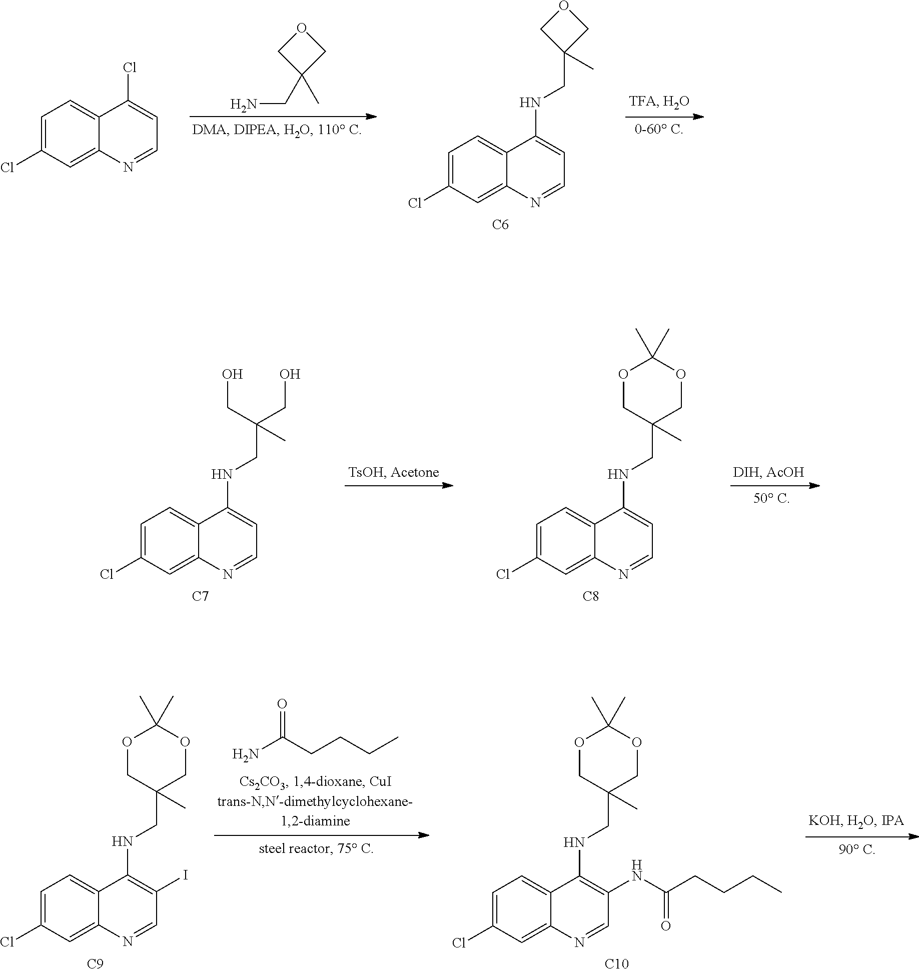

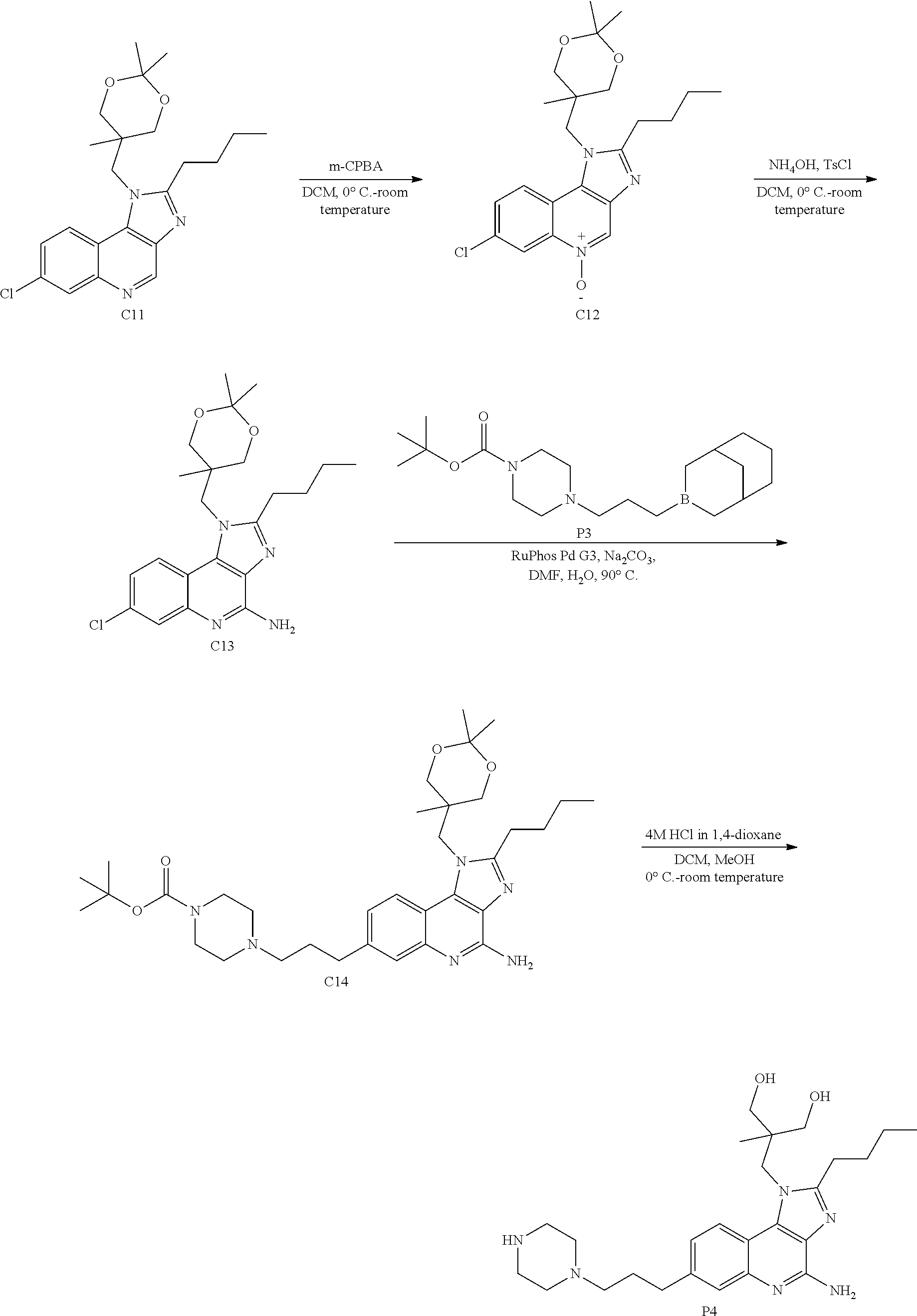

Step 1. Preparation of 7-chloro-N-((3-methyloxetan-3-yl)methyl) quinolin-4-amine (C6)

The following reaction was conducted in 5 batches in parallel then combined for work-up. To a solution of 4,7-dichloroquinoline (CAS: 86-98-6; 50.0 g, 0.252 mol) in DMA (200 mL) was added (3-methyloxetan-3-yl) methanamine (CAS: 153209-97-3; 28.0 g, 0.277 mol), DIPEA (98.0 g, 0.758 mol) and H₂O (60 mL) to provide the first batch. The reaction mixture of the first batch was stirred at 110° C. for 72 hours then cooled to room temperature.

The additional 4 batches of the same reaction were conducted with 4,7-dichloroquinoline (CAS: 86-98-6; 50.0 g, 0.252 mol) and 1 batch of the same reaction was conducted with 4,7-dichloroquinoline (CAS: 86-98-6; 10.0 g, 0.0505 mol). The 5 batches were combined then diluted with H₂O (1.5 L) dropwise. The reaction mixture was filtered and dried in vacuo to provide C6 (200. g, 71.8% yield) as a white solid. The solid was used directly in the next step without further purification. (LCMS)(M+H)⁺ 264.55. ¹H NMR (400 MHz, (CD₃)₂SO) δ 8.31 (d, 1H), 8.28 (d, 1H), 7.72 (d, 1H), 7.38 (dd, 1H), 7.17 (t, 1H), 6.48 (d, 1H), 4.42 (d, 2H), 4.18 (d, 2H), 3.40 (d, 2H), 1.26 (s, 3H).

### Step 2. Preparation of 2-(((7-chloroquinolin-4-yl)amino)methyl)-2-methylpropane-1,3-diol (C7)

The following reaction was conducted in 5 batches in parallel then combined for work-up. At 0° C., to a solution of C6 (47.5 g, 0.181 mol) in H₂O (285 mL) was slowly added TFA (190 mL) to give the first batch. The reaction mixture of the first batch was heated to 60° C. and stirred for 1 hour.

The additional 3 batches of the same reaction were conducted with C6 (47.5 g, 0.181 mol) and 1 batch of the same reaction was conducted with C6 (10.0 g, 0.0381 mol). The 5 batches were combined then cooled to room temperature and concentrated in vacuo. The residue was diluted with MeOH (1.0 L) then concentrated in vacuo again before 5% aqueous NaHCO₃ (1.5 L) was slowly added. The reaction mixture was stirred at room temperature for 16 hours before filtration. The filter cake was collected then the white solid was stirred in H₂O (1.0 L) at room temperature overnight. The suspension was filtered, and the filter cake was collected then dried in vacuo to provide C7 (190. g, 88.9% yield) as a white solid. The solid was used directly in the next step without additional purification. (LCMS)(M+H)⁺ 281.15. ¹H NMR (400 MHz, (CD₃)₂SO) δ 8.46 (d, 1H), 8.28 (d, 1H), 8.24-8.17 (m, 1H), 7.86 (d, 1H), 7.63 (dd, 1H), 6.83 (d, 1H), 4.81 (br s, 2H), 3.44-3.29 (m, 6H), 0.87 (s, 3H).

Step 3. Preparation of 7-chloro-N-((2.2,5-trimethyl-1,3-dioxan-5-yl)methyl) quinolin-4-amine (C8)

The following reaction was conducted in 5 batches in parallel then combined for work-up. To a solution of C7 (45.0 g, 0.160 mol) in acetone (1.0 L) was added TsOH (30.4 g, 0.176 mol) to provide the first batch. The reaction mixture of the first batch was stirred at room temperature for 16 hours.

The additional 3 batches of the same reaction were conducted with C7 (45.0 g, 0.160 mol) and 1 batch of the same reaction was conducted with C7 (10.0 g, 0.0356 mol). The 5 batches were combined then filtered. The filter cake was washed with acetone (1.2 L) then collected. The solid was diluted with DCM (4.0 L) and saturated NaHCO₃ (4.0 L). The suspension at room temperature was stirred vigorously for 2 hours then extracted with DCM (1.0 L×3), washed with brine, dried with Na₂SO4 and concentrated in vacuo to provide C8 (179 g, 82.4% yield) as white solid. The solid was used directly in the next step without additional purification. (LCMS)(M+H)⁺ 321.10. ¹H NMR (400 MHz, (CD₃)₂SO) δ 8.42-8.37 (m, 2H), 7.78 (d, 1H), 7.50-7.44 (m, 1H), 7.30 (t, 1H), 6.77 (d, 1H), 3.60 (s, 4H), 3.47 (d, 2H), 1.38 (d, 6H), 0.85 (s, 3H).

Step 4. Preparation of 7-chloro-3-iodo-N-((2,2,5-trimethyl-1,3-dioxan-5-yl)methyl) quinolin-4-amine (C9)

The following reaction was conducted in 6 batches in parallel then combined for work-up. To a solution of C8 (42 g, 0.13 mol) in AcOH (420 mL) was added DIH (55 g, 0.14 mol) to provide the first batch. The reaction mixture of the first batch was stirred at 50° C. for 2 hours then cooled to room temperature.

The additional 3 batches of the same reaction were conducted with C8 (42 g, 0.13 mol), 1 batch of the same reaction was conducted with C8 (10. g, 0.031 mol) and 1 batch of the same reaction was conducted with C8 (1.0 g, 0.0031 mol). The 6 batches were combined then cooled to 0° C. before 5M NaOH (6.0 L) was added slowly until pH˜8. The combined basic reaction mixture was extracted with EtOAc (2.0 L×3). The combined organic layer was concentrated in vacuo then the residue was purified by column chromatography (silica gel, EtOAc: petroleum ether, 0:100 to 30:70) to provide C9 (150 g, 60.2% yield) as a yellow solid. (LCMS)(M+H)+447.15. ¹H NMR (400 MHz, (CD₃)₂SO) δ 8.81 (s, 1H), 8.34-8.25 (m, 1H), 7.91-7.89 (m, 1H), 7.57-7.48 (m, 1H), 5.25 (t, 1H), 3.75 (d, 2H), 3.63-3.52 (m, 4H), 1.33 (s, 3H), 1.21 (s, 3H), 0.90 (s, 3H).

Step 5. Preparation of N-(7-chloro-4-(((2,2,5-trimethyl-1,3-dioxan-5-yl)methyl)amino) quinolin-3-yl)pentanamide (C10)

The following reaction was conducted in 8 batches in parallel then combined for work-up. To a solution of C9 (20.0 g, 44.8 mmol) in 1,4-dioxane (200 mL) was added pentanamide (CAS: 626-97-1; 7.70 g, 76.2 mmol), Cs₂CO₃ (29.2 g, 89.6 mmol), CuI (1.70 g, 8.96 mmol), and then trans-N, N′-dimethylcyclohexane-1,2-diamine (CAS: 67579-81-1; 1.30 g, 8.96 mmol) successively to form the first batch. The reaction mixture of the first batch was degassed with N₂ gas for 3 minutes then sealed and stirred at 75° C. for 16 hours in a steel reactor then cooled to room temperature.

The additional 6 batches of the same reaction were conducted with C9 (20.0 g, 44.8 mmol) and 1 batch of the same reaction was conducted with C9 (10.0 g, 22.4 mmol). The 8 batches were combined then filtered through a celite pad. The filtrate was concentrated in vacuo then the residue was purified by column chromatography (silica gel, EtOAc: petroleum ether, 0-100% EtOAc) to provide C10 (110. g, 78.0% yield) as a yellow solid. (LCMS)(M+2H)+422.2.

Step 6. Preparation of 2-butyl-7-chloro-1-((2,2,5-trimethyl-1,3-dioxan-5-yl)methyl)-1H-imidazo[4,5-c]quinoline (C11)

The following reaction was conducted in 5 batches in parallel then combined for work-up. To a solution of C10 (30.0 g, 71.4 mmol) in IPA (1100 mL) was added 3M KOH in H₂O (214 mL, 643 mmol) and stirred at 90° C. for 16 hours then cooled to room temperature to provide the first batch.

The additional 2 batches of the same reaction were conducted with C10 (30.0 g, 71.4 mmol) and 2 batches of the same reaction were conducted with C10 (10.0 g, 23.8 mmol). The 5 batches were combined then poured into H₂O (7 L). The resultant white suspension was stirred at room temperature for 1 hour then filtered to provide C11 (100. g, 95.0% yield) as a white solid. The solid was used directly in the next step without further purification. (LCMS)(M+H)⁺ 402.1. ¹H NMR (400 MHz, (CD₃)₂SO) δ 9.17 (s, 1H), 8.72 (d, 1H), 8.14 (d, 1H), 7.62 (dd, 1H), 4.88 (d, 2H), 3.92-3.50 (m, 4H), 3.11 (t, 2H), 1.87-1.75 (m, 2H), 1.41-1.37 (m, 8H), 0.95 (t, 3H), 0.57 (s, 3H).

Step 7. Preparation of 2-butyl-7-chloro-1-((2,2,5-trimethyl-1,3-dioxan-5-yl)methyl)-1H-imidazo[4,5-c]quinoline 5-oxide (C12)

The following reaction was conducted in 4 batches in parallel then combined for work-up. At 0° C., to a solution of C11 (30.0 g, 74.6 mmol) in DCM (750 mL) was added m-CPBA (19.3 g, 112 mmol) to form the first batch. The reaction mixture of the first batch was warmed to room temperature slowly then stirred for 16 hours.

The additional 2 batches of the same reaction were conducted with C11 (30.0 g, 74.6 mmol) and 1 batch of the same reaction was conducted with C11 (10.0 g, 24.9 mmol). The 4 batches were combined then 5% NaHCO₃ solution (1.5 L) was added and stirred at room temperature for 2 hours. The layers were separated then the organic layer was washed with 5% NaHCO₃ (1.5 L×2), brine and dried with Na₂SO₄. The dried organic layer was filtered and concentrated in vacuo. To the brown oil was added EtOAc (1.0 L) then the solution was concentrated in vacuo to form a foam. To the foam was added MTBE (1.0 L) then the solution was concentrated in vacuo to form a brown solid. To the solid was added MTBE (0.5 L) and the reaction mixture was stirred for 16 hours then filtered. The filter cake was collected to provide C12 (80.0 g, 76.9% yield) as a brown solid. The solid was used directly in the next step without further purification. (LCMS)(M+H)⁺ 418.0. 1H NMR (400 MHz, (CD₃)₂SO) δ 9.05 (s, 1H), 8.81-8.74 (m, 2H), 7.77 (d, 1H), 4.85 (d, 2H), 3.89-3.54 (m, 4H), 3.08-3.06 (m, 2H), 1.84-1.74 (m, 2H), 1.40-1.36 (m, 8H), 0.94 (t, 3H), 0.59 (s, 3H).

Step 8. Preparation of 2-butyl-7-chloro-1-((2,2,5-trimethyl-1,3-dioxan-5-yl)methyl)-1H-imidazo[4,5-c]quinolin-4-amine (C13)

The following reaction was conducted in 4 batches in parallel then combined for work-up. At 0° C., to a solution of C12 (20. g, 48 mmol) in DCM (800 mL) was added 28% NH₄OH in H₂O (150 mL) and TsCl (11 g, 57 mmol) successively to form the first batch. The reaction mixture of the first batch was warmed to room temperature slowly then stirred for 16 hours.

The additional 3 batches of the same reaction were conducted with C12 (20. g, 48 mmol). The 4 batches were combined then washed with saturated NaHCO₃ (2 L×2) and brine (2 L×2). The organic layer was concentrated in vacuo then the residue was slurried in IPA (650 mL) at room temperature for 16 hours before the suspension was filtered. The filter cake was collected to provide C13 (66 g, 83% yield) as a yellow solid. The solid was used directly in the next step without further purification. (LCMS)(M+H)⁺ 417.2. ¹H NMR (400 MHz, (CD₃)₂SO) δ 8.35 (d, 1H), 7.54 (d, 1H), 7.16 (dd, 1H), 6.71 (s, 2H), 4.83-4.73 (m, 2H), 3.90-3.35 (m, 4H), 3.04 (br d, 2H), 1.75 (br s, 2H), 1.46-1.36 (m, 8H), 0.94 (t, 3H), 0.57 (s, 3H).

Step 9. Preparation of tert-butyl 4-(3-(4-amino-2-butyl-1-((2,2,5-trimethyl-1,3-dioxan-5-yl)methyl)-1H-imidazo[4.5-c]quinolin-7-yl)propyl) piperazine-1-carboxylate (C14)

The following reaction was conducted in 7 batches in parallel then combined for work-up. To a solution of C13 (10. g, 24 mmol) in DMF (300 mL) was added Na2CO₃ (7.5 g, 72 mmol), RuPhos Pd G3 (4.0 g, 4.8 mmol), H₂O (40 mL) and P3 (120 mL) successively to provide the first batch. The reaction mixture of the first batch was degassed with N₂ gas then stirred at 90° C. for 16 hours under N₂ gas.

The additional 5 batches of the same reaction were conducted with C13 (10. g, 24 mmol) and 1 batch of the same reaction was conducted with C13 (5.0 g, 0.12 mol). The 7 batches were combined then filtered through a celite pad. The filtrate was diluted with H₂O (5 L) then extracted with EtOAc (2 L×3). The combined organic layer was washed with brine (2 L ×3), dried with Na₂SO4 and concentrated in vacuo. The residue was purified by column chromatography (silica gel, MeOH: DCM, 0:100 to 10:90) to provide a yellow oil that was slurried in MTBE (800 mL) at room temperature for 16 hours then was filtered. The filter cake was collected to provide C14 (63 g, 66% yield) as an off-white solid. (LCMS)(M+H)⁺ 609.5. ¹H NMR (400 MHz, (CD₃)₂SO) δ 8.23 (d, 1H), 7.38 (d, 1H), 7.06 (dd, 1H), 6.40 (s, 2H), 4.77 (br s, 2H), 3.92-3.51 (m, 4H), 3.41-3.34 (m, 1H), 3.29-3.26 (m, 2H), 3.12-2.89 (m, 2H), 2.68 (t, 2H), 2.36-2.35 (m, 6H), 1.84-1.67 (m, 4H), 1.44-1.36 (m, 18H), 0.94 (t, 3H), 0.58 (s, 3H).

Step 10. Preparation of 2-((4-amino-2-butyl-7-(3-(piperazin-1-yl)propyl)-1H-imidazo[4,5-c]quinolin-1-yl)methyl)-2-methylpropane-1.3-diol hydrochloride (P4)

The following reaction was conducted in 7 batches in parallel then combined for work-up. At 0° C., to a solution of C14 (10. g, 16 mmol) in (1:1) MeOH:DCM (260 mL) was added 4M HCl in 1,4-dioxane (200 mL) then stirred 16 hours at room temperature to provide the first batch.

The additional 5 batches of the same reaction were conducted with C14 (10. g, 16 mmol) and 1 batch of the same reaction was conducted with C14 (3.0 g, 0.0049 mmol). The 7 batches were combined then concentrated in vacuo. The residue was slurried in EtOAc (500 mL) at room temperature for 2 hours then filtered. The filter cake was collected and dried in vacuo to provide P4 (57 g, >99% yield) as an off-white solid. The solid was used directly in the next step without additional purification. (LCMS)(M+H)⁺ 469.35. 1H NMR (400 MHz, D₂O) δ 8.38 (d, 1H), 7.52 (s, 1H), 7.45 (dd, 1H), 4.76-4.67 (m, 1H), 4.56-4.42 (m, 1H), 3.79-3.32 (m, 14H), 3.06 (t, 2H), 2.94 (t, 2H), 2.27-2.16 (m, 2H), 1.92-1.82 (m, 2H), 1.56-1.45 (m, 2H), 1.01 (t, 3H), 0.63 (s, 3H).

### Preparation P5

### Tert-butyl (2-(2-(2-(2-(chlorosulfonyl)ethoxy)ethoxy)ethoxy)ethyl)carbamate (P5)

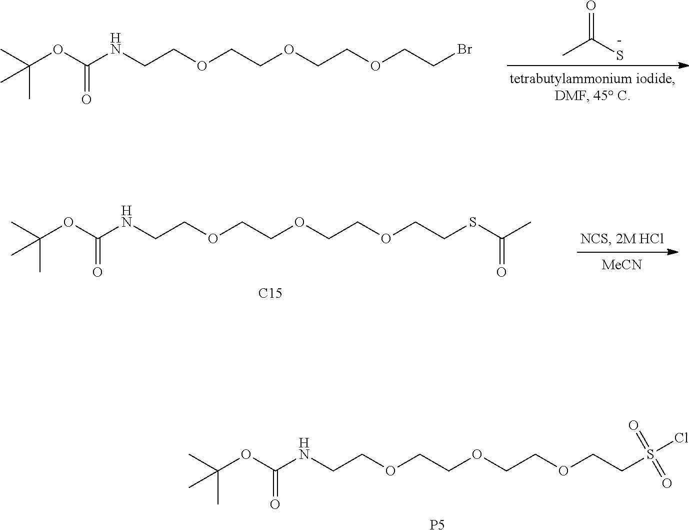

Step 1. Preparation of S-(2.2-dimethyl-4-oxo-3.8,11,14-tetraoxa-5-azahexadecan-16-yl) ethanethioate (C15)

Under N₂ gas, to a stirred solution of tert-butyl (2-(2-(2-(2-bromoethoxy) ethoxy) ethoxy)ethyl) carbamate (CAS: 1076199-21-7; 1.00 g, 2.81 mmol) and tetrabutylammonium iodide (0.104 g, 0.281 mmol) in DMF (25 mL) was added potassium thioacetate (CAS: 10387-40-3; 0.641 g, 5.61 mmol). The reaction mixture was heated to 45° C. and stirred for 2 hours then poured into ice-water (30 mL). The diluted reaction mixture was extracted with EtOAC (30 mL×3) then the combined organic layer was washed with 1M HCl (30 mL), saturated NaHCO₃ (30 mL) and brine (30 mL). The organic layer was dried over Na₂SO4, filtered, and concentrated in vacuo to provide C15 (1.10 g, >99% yield) as brown oil. The oil was used directly in the next step without further purification. (LCMS)(M+23H)⁺ 374.3.

Step 2. Preparation of tert-butyl (2-(2-(2-(2-(chlorosulfonyl)ethoxy)ethoxy)ethoxy)ethyl)carbamate (P5)

At 0-5° C., to a stirred solution of C15 (0.550 g, 1.56 mmol) in MeCN (10 mL) was added aqueous 2M HCl (114 mg, 3.13 mmol) then NCS (418 mg, 3.13 mmol). The reaction mixture was warmed to room temperature and stirred for 2.5 hours. After the stir, the reaction mixture was extracted with the EtOAc (20 mL×3). The combined organic layer was washed with saturated NaHCO₃ (20 mL×3) and brine (20 mL×3). The organic layer was dried with Na₂SO4, filtered and concentrated in vacuo to provide P5 (0.160 g, 27.2% yield) as yellow gum. The gum was used directly in the next step without further purification.

### Preparation P6

### Tert-butyl(S)-1,14-dioxo-17-palmitamido-4,7,10-trioxa-13-azaoctadecan-18-oate (P6)

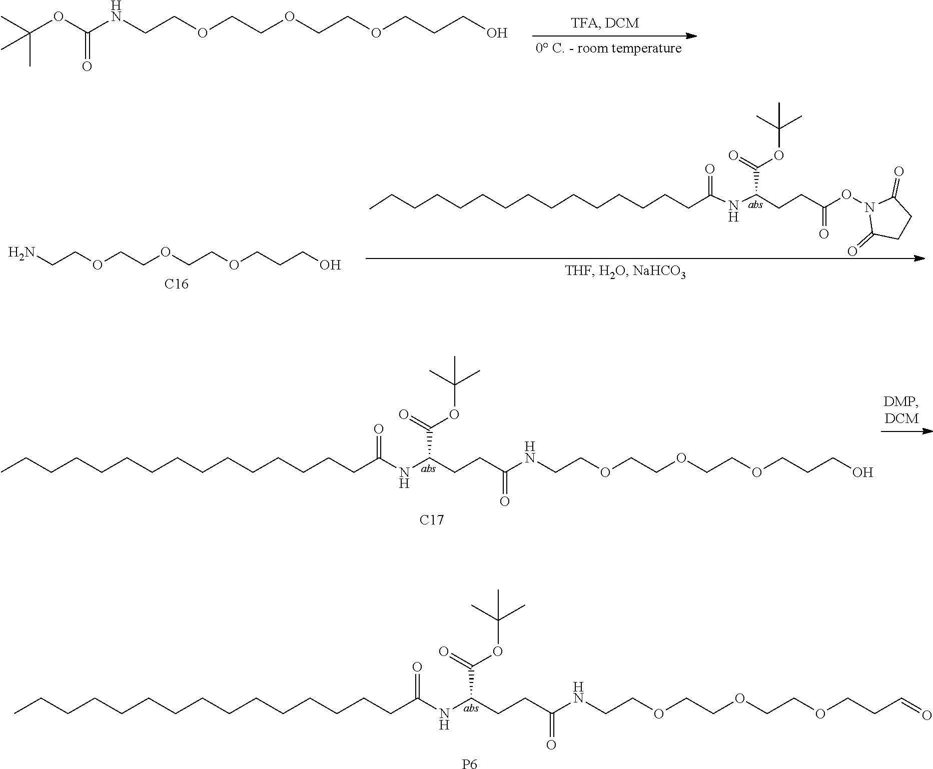

Step 1. Preparation of 3-(2-(2-(2-aminoethoxy)ethoxy)ethoxy)propan-1-ol trifluoroacetate (C16)

At 0° C. under N₂ gas, to a stirred solution of tert-butyl (2-(2-(2-(3-hydroxypropoxy) ethoxy) ethoxy)ethyl) carbamate (CAS: 1818885-72-1; 0.200 g, 0.586 mmol) in DCM (2 mL) was added TFA (2 mL). The reaction mixture was warmed to room temperature then stirred for 1 hour before the reaction mixture was concentrated in vacuo to provide C16 (325 mg, >99% yield) as a colorless oil. The oil was used directly in the next step without additional purification.

Step 2. Preparation of tert-butyl(S)-1-hydroxy-14-oxo-17-palmitamido-4,7,10-trioxa-13-azaoctadecan-18-oate (C17)

To a stirred solution of C16 (0.700 g, 1.31 mmol) in THF (6 mL) and H₂O (6 mL) was added NaHCO₃ (468 mg, 5.57 mmol) then 1-(tert-butyl) 5-(2,5-dioxopyrrolidin-1-yl) palmitoyl-L-glutamate (CAS: 204521-63-1; 0.600 g, 1.11 mmol) successively at room temperature. The reaction mixture was stirred for 16 hours at room temperature under N₂ gas before the yellow reaction mixture was diluted with EtOAc (20 mL) and washed with saturated NaHCO₃ (20 mL). The organic layer was dried with Na₂SO₄, filtered, and concentrated in vacuo. The yellow oil was purified by (silica gel, MeOH: DCM, 0-7% MeOH) to provide C17 (0.600 g, 85.4% yield) as a white solid. (LCMS)(M+H)⁺ 631.4. ¹H NMR (400 MHz, CDCl₃) δ 7.34 (t, 1H), 6.61 (d, 1H), 4.44-4.36 (m, 1H), 3.79 (t, 2H), 3.71 (t, 2H), 3.65-3.57 (m, 8H), 3.52 (t, 2H), 3.46-3.37 (m, 2H), 2.36-2.06 (m, 6H), 2.02-1.90 (m, 1H), 1.86-1.79 (m, 2H), 1.66-1.55 (m, 2H), 1.45 (s, 9H), 1.32-1.20 (m, 24H), 0.95-0.87 (t, 3H).

Step 3. Preparation of tert-butyl(S)-1,14-dioxo-17-palmitamido-4,7,10-trioxa-13-azaoctadecan-18-oate (P6)

To a stirred solution of C17 (0.200 g, 0.269 mmol) in DCM (4 mL) was added DMP (229 mg, 0.539 mmol) at room temperature then was stirred for 2 hours. The yellow reaction mixture was filtered then the filtrate was washed with saturated NaHCO₃ (10 mL), dried with Na₂SO4, filtered and concentrated in vacuo to provide P6 (0.200 g, >99% yield) as a white waxy solid. The solid was used directly in the next step without further purification. 1H NMR (400 MHz, (CD₃)₂SO) δ 9.64 (t, 1H), 8.08-7.99 (m, 1H), 7.91-7.84 (m, 1H), 4.08-3.99 (m, 1H), 3.72 (t, 2H), 3.50-3.48 (m, 8H), 3.40-3.35 (m, 2H), 3.17 (q, 2H), 2.60 (td, 2H), 2.10 (dt, 4H), 1.90-1.81 (m, 1H), 1.78-1.66 (m, 1H), 1.52-1.42 (m, 2H), 1.38 (s, 9H), 1.23 (s, 24H), 0.85 (t, 3H).

### Preparation P7

### (S)-2-(1-(5-(Tert-butoxy)-5-oxo-4-palmitamidopentanoyl)-4-hydroxypiperidin-4-yl) acetic acid (P7)

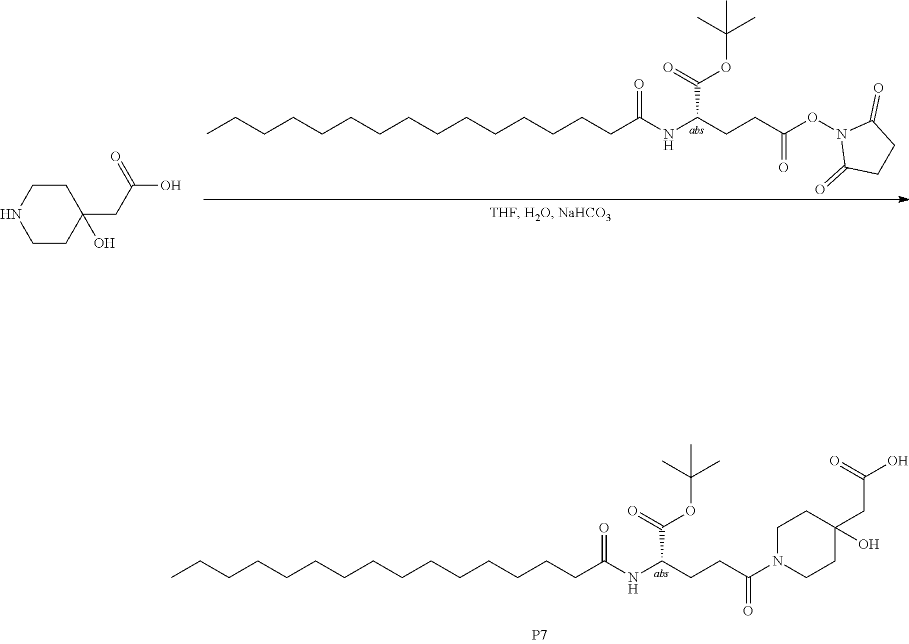 [SMILES: O=C(O)CC1(O)CCNCC1 ; CCCCCCCCCCCCCCCC(=O)N[C@@H](CCC(=O)ON1C(=O)CCC1=O)C(=O)OC(C)(C)C ; CCCCCCCCCCCCCCCC(=O)N[C@@H](CCC(=O)N1CCC(O)(CC(=O)O)CC1)C(=O)OC(C)(C)C]

Preparation of(S)-2-(1-(5-(tert-butoxy)-5-oxo-4-palmitamidopentanoyl)-4-hydroxypiperidin-4-yl) acetic acid (P7)

To a stirred solution of 2-(4-hydroxypiperidin-4-yl) acetic acid (CAS: 328401-29-2; 0.350 g, 0.730 mmol) in THF (2 mL) and H₂O (2 mL) was added NaHCO₃ (0.600 g, 7.14 mmol) to PH˜8 then 1-(tert-butyl) 5-(2,5-dioxopyrrolidin-1-yl) palmitoyl-L-glutamate (CAS: 204521-63-1; 0.393 g, 0.730 mmol) successively at room temperature. The reaction mixture was stirred at room temperature under N₂ gas for 2 hours then was acidified with aqueous 1M HCl to PH˜4. The acidic reaction mixture was extracted with EtOAc (20 mL×3) then the combined organic layer was dried with Na₂SO₄, filtered, and concentrated in vacuo. The yellow oil was purified by column chromatography (silica gel, MeOH: DCM, 0-7% MeOH) to provide P7 (0.350 g, 82.3% yield) as a colorless gum. (LCMS)(M+H)⁺ 583.5. 1H NMR (400 MHz, CDCl₃) δ 6.75-6.61 (m, 1H), 4.46 (br s, 1H), 4.33 (t, 1H), 3.64-3.55 (m, 1H), 3.47-3.39 (m, 1H), 3.07 (q, 1H), 2.54-2.31 (m, 4H), 2.27-2.10 (m, 3H), 2.05-1.90 (m, 1H), 1.84-1.70 (m, 2H), 1.65-1.55 (m, 2H), 1.46 (s, 12H), 1.30-1.22 (m, 24H), 0.87 (t, 3H).

### Preparation P8

### 2-((4-Amino-2-(ethoxymethyl)-7-(3-(piperazin-1-yl)propyl)-1H-imidazo[4,5-c]quinolin-1-yl)methyl)-2-methylpropane-1,3-diol hydrochloride (P8)

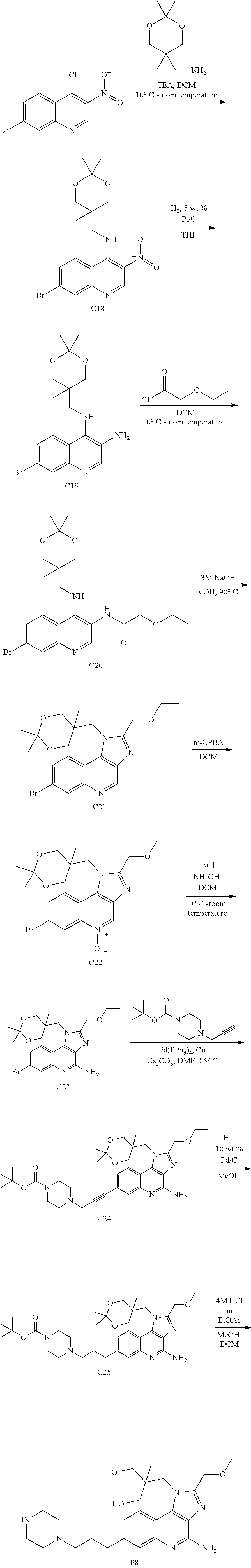

Step 1. Preparation of 7-bromo-3-nitro-N-((2,2,5-trimethyl-1,3-dioxan-5-yl)methyl) quinolin-4-amine (C18)

At 10° C., to a yellow suspension of 7-bromo-4-chloro-3-nitroquinoline (CAS: 723280-98-6; 49.0 g, 0.170 mol) and TEA (34.5 g, 341 mmol) in DCM (820 mL) was added a solution of (2,2,5-trimethyl-1,3-dioxan-5-yl) methanamine (CAS: 493320-4; 27.1 g, 0.170 mol) in DCM (15 mL) then stirred at room temperature for 2 hours. The yellow reaction mixture changed from a yellow suspension to a clear solution. After the stir, the yellow reaction solution was diluted with DCM (300 mL) then washed with brine (200 mL), dried with Na₂SO₄, filtered and concentrated in vacuo. The yellow solid was diluted with DCM (100 mL) and stirred at room temperature for 20 minutes. The suspension was filtered, and the filter cake was collected then dried further to provide C18 (60.0 g) as a yellow solid.

The filtrate was concentrated in vacuo to provide a brown solid which was diluted with DCM (20 mL) then stirred at room temperature for 20 minutes. The yellow reaction suspension was filtered and washed with DCM (5 mL). The filter cake was collected then dried further to provide C18 (4.56 g) as a yellow solid.

The two batches of the desired intermediate were combined to provide C18 (64.6 g, 92.3% yield) as a yellow solid. The solid was used directly in the next step without additional purification. (LCMS)(M+H)⁺ 411.9. ¹H NMR (400 MHz, (CD₃)₂SO) δ 9.24-9.18 (m, 1H), 9.14 (s, 1H), 8.50 (d, 1H), 8.12 (d, 1H), 7.77 (dd, 1H), 3.93 (d, 2H), 3.65 (d, 2H), 3.55 (d, 2H), 1.35 (s, 3H), 1.22 (s, 3H), 0.91 (s, 3H).

Step 2. Preparation of 7-bromo-N4-((2.2.5-trimethyl-1,3-dioxan-5-yl)methyl) quinoline-3.4-diamine (C19)

To a suspension of C18 (30.0 g, 73.1 mmol) in THF (250 mL) was added Pt/C (4.28 g, 1.10 mmol, 5 wt %). The reaction mixture was stirred under a balloon of H2 at room temperature for 32 hours then was filtered through a pad of celite and washed with THF (600 mL). The filtrate was concentrated in vacuo then diluted with MeCN (50 mL) and stirred for 15 minutes. The reaction mixture was filtered and washed with MeCN (5 mL×2). The filter cake was collected and concentrated in vacuo to give C19 (21.0 g) as a yellow-green solid.

The filtrate was concentrated in vacuo then diluted with DCM (15 mL) and stirred at room temperature for 20 minutes. The yellow suspension was filtered and washed with DCM (5 mL). The filter cake was collected and dried to give C19 (4.61 g) as a brown solid.

The two batches of the desired intermediate were combined to provide C19 (25.6 g, 92.1% yield) as a solid. The solid was used directly in the next step without additional purification. (LCMS)(M)+380.1. ¹H NMR (400 MHz, (CD₃)₂SO) δ 8.37 (s, 1H), 8.01 (d, 1H), 7.89 (d, 1H), 7.44 (dd, 1H), 5.27 (s, 2H), 4.58 (t, 1H), 3.66 (d, 2H), 3.55 (d, 2H), 3.22 (d, 2H), 1.35 (s, 3H), 1.27 (s, 3H), 0.87 (s, 3H).

Step 3. Preparation of N-(7-bromo-4-(((2,2,5-trimethyl-1,3-dioxan-5-yl)methyl)amino) quinolin-3-yl)-2-ethoxyacetamide (C20)

At 0° C. under N₂ gas, to a solution of C19 (12.0 g, 31.6 mmol) in DCM (240 mL) was added 2-ethoxyacetyl chloride (CAS: 14077-58-8; 3.87 g, 31.6 mmol) dropwise. The mixture was stirred from 0° C. to room temperature over 1 hour. The yellow reaction mixture was poured into saturated NaHCO₃ (300 mL) and extracted with DCM (150 mL×2). The combined organic layer was washed with brine (150 mL), dried with Na₂SO₄, filtered and concentrated in vacuo. The brown oil was purified by column chromatography (silica gel, EtOAc, 100%) to provide C20 (19.0 g, 60.7% yield) as a yellow gum. (LCMS)(M)+466.1. ¹H NMR (400 MHz, (CD₃)₂SO) δ 9.57 (s, 1H), 8.38-8.36 (m, 1H), 8.26 (d, 1H), 8.07-8.02 (m, 1H), 7.61 (dd, 1H), 5.75 (t, 1H), 4.11 (s, 2H), 3.72-3.51 (m, 8H), 1.38-1.29 (m, 3H), 1.28-1.16 (m, 6H), 0.81 (s, 3H).

Step 4. Preparation of 7-bromo-2-(ethoxymethyl)-1-((2.2,5-trimethyl-1,3-dioxan-5-yl)methyl)-1H-imidazo[4,5-c]quinoline (C21)

The following reaction was conducted in two batches then combined for purification. Under N₂ gas, to a solution of C20 (19.2 g, 41.2 mmol) in EtOH (150 mL) was added aqueous 3M NaOH (20.6 mL) to form the first batch. The reaction mixture of the first batch was stirred at 90° C. for 2 hours.

A second batch of the same reaction was conducted with C20 (10.0 g, 21.4 mmol). The two batches were combined then concentrated in vacuo. The residue was diluted with H₂O (100 mL) and EtOAc (30 mL) then stirred at room temperature for 15 minutes before filtration. The filter cake was washed with H₂O (20 mL) then collected. The solid was diluted with (2:1) EtOAc: petroleum ether (60 mL) and stirred for 40 minutes. The yellow suspension was filtered and washed with EtOAc (10 mL×3). The filter cake was collected again then diluted with MeCN and concentrated in vacuo to provide C21 (15.8 g, 56.3% yield) as a faint yellow solid. The solid was used directly in the next step without additional purification. (LCMS)(M+H)⁺ 449.9. 1H NMR (400 MHz, (CD₃)₂SO) δ 9.17 (s, 1H), 8.63 (d, 1H), 8.29 (d, 1H), 7.73 (dd, 1H), 5.25-4.89 (m, 3H), 4.79-4.57 (m, 1H), 3.89-3.46 (m, 6H), 1.35 (d, 6H), 1.10 (t, 3H), 0.53 (s, 3H).

Step 5. Preparation of 4-amino-7-bromo-2-(ethoxymethyl)-1-((2,2,5-trimethyl-1,3-dioxan-5-yl)methyl)-1H-imidazo[4.5-c]quinoline 5-oxide (C22)

Under N₂ gas, to a solution of C21 (15.8 g, 35.1 mmol) in DCM (450 mL) was added m-CPBA (8.56 g, 42.2 mmol) then was stirred at room temperature for 16 hours. The reaction solution was diluted with DCM (500 mL) and washed with saturated NaHCO₃ (200 mL) then brine (200 mL). The organic layer was dried with Na₂SO₄, filtered, and concentrated in vacuo. The yellow solid was suspended in petroleum ether (60 mL) and DCM (20 mL) then stirred at room temperature for 15 minutes. The suspension was filtered then the filter cake was collected to provide C22 (14.5 g, 88.7%) as a yellow solid. (LCMS)(M)+464.2. ¹H NMR (400 MHz, (CD₃)₂SO) δ 9.11 (s, 1H), 8.95 (d, 1H), 8.73 (d, 1H), 7.93 (dd, 1H), 5.19-4.93 (m, 3H), 4.73-4.61 (m, 1H), 3.93-3.50 (m, 6H), 1.40 (d, 6H), 1.16 (t, 3H), 0.60 (s, 3H).

Step 6. Preparation of 7-bromo-2-(ethoxymethyl)-1-((2,2,5-trimethyl-1,3-dioxan-5-yl)methyl)-1H-imidazo[4,5-c]quinolin-4-amine (C23)

At 0° C., to a solution of C22 (14.5 g, 31.2 mmol) in DCM (250 mL) was added NH₄OH (78.0 g, 623 mmol) then TsCl (7.13 g, 37.4 mmol) portion wise over 20 minutes. The reaction mixture was warmed to room temperature and stirred for 16 hours. The white suspension was diluted with DCM (400 mL), washed with saturated NaHCO₃ (300 mL) then brine (200 mL). The organic layer was dried with Na₂SO₄, filtered and concentrated in vacuo. The yellow solid was suspended in DCM (40 mL) and petroleum ether (30 mL) then stirred at room temperature for 15 minutes before filtration. The filter cake was washed with DCM (5 mL×2) then collected and dried to provide C23 (12.5 g, 86.6% yield) as a light-yellow solid. (LCMS)(M+2H)+465.0. ¹H NMR (400 MHz, (CD₃)₂SO) δ 8.31 (d, 1H), 7.71 (d, 1H), 7.39-7.24 (m, 1H), 6.87 (s, 2H), 5.18-4.80 (m, 3H), 4.68-4.56 (m, 1H), 3.91-3.48 (m, 6H), 1.41 (d, 6H), 1.14 (t, 3H), 0.58 (s, 3H).

Step 7. Preparation of tert-butyl 4-(3-(4-amino-2-(ethoxymethyl)-1-((2,2,5-trimethyl-1,3-dioxan-5-yl)methyl)-1H-imidazo[4,5-c]quinolin-7-yl) prop-2-yn-1-yl) piperazine-1-carboxylate (C24)

Under argon gas, to a solution of C23 (6.00 g, 12.9 mmol), tert-butyl 4-(prop-2-yn-1-yl) piperazine-1-carboxylate (CAS: 199538-99-3; 3.49 g, 15.5 mmol) in DMF (240 mL) was added Pd (PPh₃)₄ (1.50 g, 1.29 mmol), CuI (0.493 g, 2.59 mmol), and Cs₂CO₃ (21.1 g, 64.7 mmol) at room temperature. The reaction mixture was heated to 85° C. and stirred for 16 hours then cooled to room temperature. The cooled reaction mixture was diluted with H₂O (700 mL) then stirred for 3 minutes. The yellow suspension was filtered then the filter cake was washed with H₂O (50 mL×2). The aqueous filtrate was discarded. The filter cake was then rinsed with DCM (˜300 mL) and the filtrate was dried with Na₂SO₄, filtered, and concentrated in vacuo. The brown gum was purified by column chromatography (silica gel, MeOH: EtOAc, 0-6% MeOH) to provide an impure C24 as a brown solid which was suspended in MeCN (20 mL) and petroleum ether (10 mL) then stirred at room temperature for 15 minutes before filtration. The filter cake was washed with MeCN (5 mL×2) then collected and dried to provide C24 (3.60 g, 45.8% yield) as a white solid. (LCMS)(M+H)⁺ 607.2. 1H NMR (400 MHz, CDCl₃) δ 8.10 (d, 1H), 7.88-7.86 (m, 1H), 7.31 (dd, 1H), 5.51 (br s, 2H), 5.22-4.66 (m, 4H), 3.84-3.60 (m, 4H), 3.50 (t, 4H), 2.61 (t, 4H), 1.74 (br s, 4H), 1.54-1.48 (m, 6H), 1.47 (br s, 9H), 1.28-1.22 (m, 3H), 0.63 (s, 3H).

Step 8. Preparation of tert-butyl 4-(3-(4-amino-2-(ethoxymethyl)-1-((2.2.5-trimethyl-1,3-dioxan-5-yl)methyl)-1H-imidazo[4,5-c]quinolin-7-yl)propyl) piperazine-1-carboxylate (C25)

A mixture of C24 (5.3 g, 8.7 mmol) in MeOH (270 mL) was added Pd/C (1.6 g, 1.6 mmol, 10 wt %). The reaction mixture was degassed with H₂ gas 3 times then stirred at room temperature under a balloon of H2 for 20 hours. The suspension was filtered through a pad of celite then the filter cake was washed with MeOH (50 mL). The filtrate was concentrated in vacuo then the gray gum was purified by column chromatography (silica gel, MeOH: EtOAc, 0-4% MeOH; then MeOH: DCM, 0-6% MeOH) to provide the impure C25 brown gum. The brown gum was suspended in petroleum ether (30 mL) and EtOAc (5 mL) then stirred at room temperature for 15 minutes before filtration. The filter cake was washed with EtOAc (5 mL×2) then was collected and dried further to provide C25 (3.8 g, 71% yield) as a white solid. (LCMS) (M+H)⁺ 611.5. 1H NMR (400 MHz, (CD₃)₂SO) δ 8.23 (d, 1H), 7.39-7.37 (m, 1H), 7.07 (dd, 1H), 6.59 (br s, 2H), 5.14-4.78 (m, 3H), 4.65-4.52 (m, 1H), 3.89-3.48 (m, 6H), 3.28-3.25 (m, 4H), 2.66 (t, 2H), 2.33-2.24 (m, 6H), 1.83-1.74 (m, 2H), 1.42-1.32 (m, 15H), 1.12 (t, 3H), 0.57 (s, 3H).

Step 9. Preparation of 2-((4-amino-2-(ethoxymethyl)-7-(3-(piperazin-1-yl)propyl)-1H-imidazo[4,5-c]quinolin-1-yl)methyl)-2-methylpropane-1,3-diol hydrochloride (P8)

To a mixture of C25 (3.80 g, 6.22 mmol) in DCM (50 mL) and MeOH (50 mL) was added 4M HCl in EtOAc (150 mL). The reaction mixture was stirred at room temperature for 2 hours then the white suspension was filtered. The filter cake was collected then lyophilized to provide P8 (3.82 g, >99% yield) as a light-brown solid. The solid was used directly in the next step without additional purification. (LCMS)(M+H)⁺ 471.3. 1H NMR (400 MHz, (CD₃)₂SO) δ 14.05 (s, 1H), 12.06 (br s, 1H), 10.07-9.78 (m, 2H), 9.30 (br s, 1H), 8.69 (d, 1H), 7.63 (s, 1H), 7.44 (d, 1H), 5.15-4.61 (m, 4H), 3.77-3.13 (m, 16H), 2.84 (t, 2H), 2.19-2.04 (m, 2H), 1.14 (t, 3H), 0.56 (s, 3H).

The Schemes described below are intended to provide a general description of the methodology employed in the preparation of the compounds of the present disclosure. Some of the compounds of the present disclosure contain a single chiral center. In the following Schemes, the general methods for the preparation of the compounds are shown either in racemic or enantioenriched form. It will be apparent to one skilled in the art that all of the synthetic transformations may be conducted in a precisely similar manner whether the materials are enantioenriched or racemic. Moreover, the resolution to the desired optically active material may take place at any desired point in the sequence using well known methods such as described herein and in the chemistry literature.

Compounds of the disclosure may be made according to the following Schemes 1-6, although alternative methodologies may also be utilized. One skilled in the art will appreciate that alternative reaction conditions to the ones illustrated in the schemes and examples may be utilized as deemed appropriate. Choices of solvents, additives such as acidic or basic catalysts, coupling agents, and indeed the reaction sequence may be changed as appropriate for a given target compound.

In some cases, compounds of described herein may contain protecting groups, which may be appended or removed by additional steps in the synthetic sequence using conditions known in the art (March's Advanced Organic Chemistry: Reactions, Mechanisms, and Structure 8th Edition or Protecting Groups, 10 Georg Thieme Verlag, 1994). Compounds at every step may be purified by standard techniques, such as column chromatography, crystallization, or reverse phase SFC or HPLC.

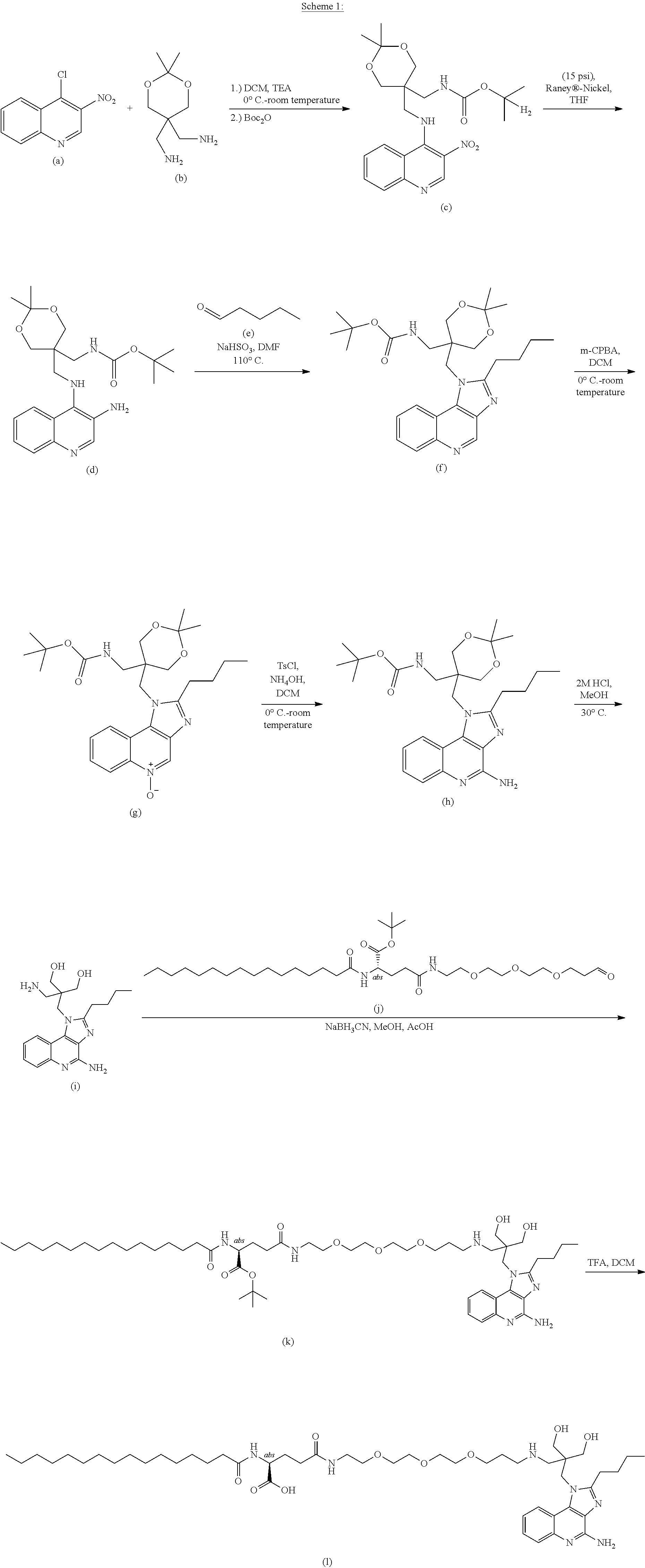

The halogen (e.g. CI)-substituted heterocycle (a) can react with the amine (b) in a solvent such as dichloromethane (DCM) and with a base like triethylamine (TEA) from 0° C. to room temperature which then can react with di-tert-butyl decarbonate (Boc2O) to provide the protected amine (c). The nitro-intermediate (c) can then be reduced through a hydrogenation with a catalyst such as Raney®-Nickel under hydrogen gas (H2) in a solvent like tetrahydrofuran (THF) to afford the aniline (d). The aniline (d) can then react with pentanal (e) and cyclize under basic conditions like sodium bisulfite (NaHSO3) in a suitable solvent such as dimethylformamide (DMF) heated from 80-110° C. then stirred from 16-24 hours to provide the tricycle (f). The tricycle (f) in a solvent such as DCM can oxidize with the treatment of 3-chloroperoxybenzoic acid (m-CPBA) with stirring from 24-48 hours at room temperature to provide the N-oxide (g). The N-oxide (g) in a solvent like DCM with a base such as ammonium hydroxide (NH₄OH) can react with p-toluenesulfonyl chloride (TsCl) from 0° C. to room temperature with stirring from 16-24 hours to afford the aniline (h). The aniline intermediate (h) can undergo deprotection under standard acidic conditions such as with hydrochloric acid (HCl) in a solvent like methanol (MeOH) and heated from 30-50° C. to form the amine (i). The amine (i) can undergo a reductive amination with the aldehyde (j) in a solvent like MeOH with a mild reducing agent such as sodium cyanoborohydride (NaBH3CN) and with a catalytic amount of acid such as acetic acid (AcOH) to afford the protected acid intermediate (k). The protected acid intermediate (k) can undergo a deprotection with acidic conditions like trifluoroacetic acid (TFA) in a solvent like DCM from 0° C. to room temperature to form the target product (I).

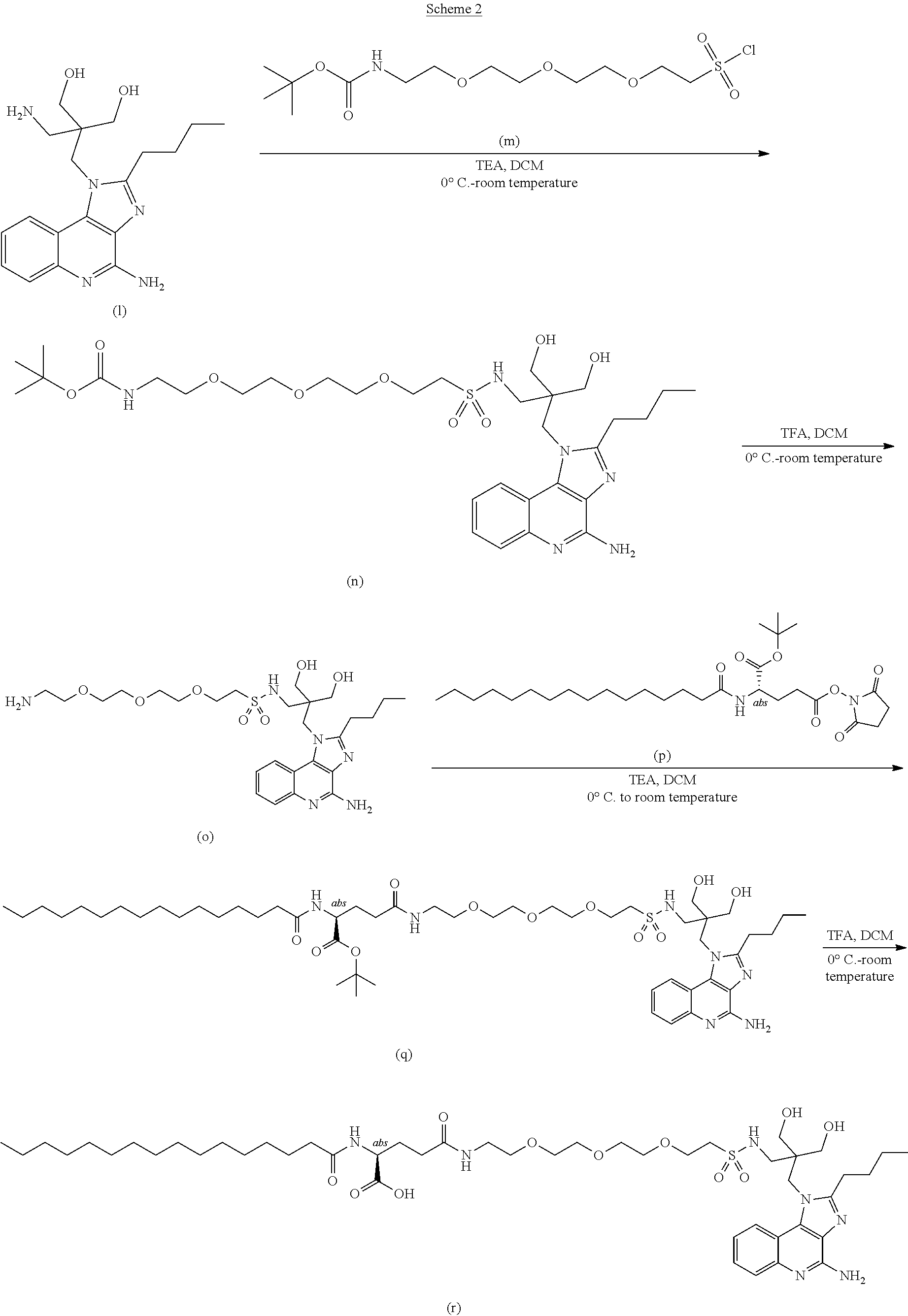

The amine (i) can react with the sulfonyl chloride (m) in a solvent like DCM and with a base like TEA from 0° C. to room temperature stirred from 16-24 hours to form the terminal protected amine intermediate (n) which can be deprotected under standard acidic conditions like with TFA in a solvent like DCM from 0° C. to room temperature to provide the terminal amine (o). The amine (o) can react with the NHS-ester (N-hydroxysuccinimide ester)(p) in a solvent like DCM and with a base like TEA from 0° C. to room temperature to afford the protected acid intermediate (q) which can be deprotected under standard acidic conditions like TFA in a solvent like DCM from 0° C. to room temperature to provide the target product (r).

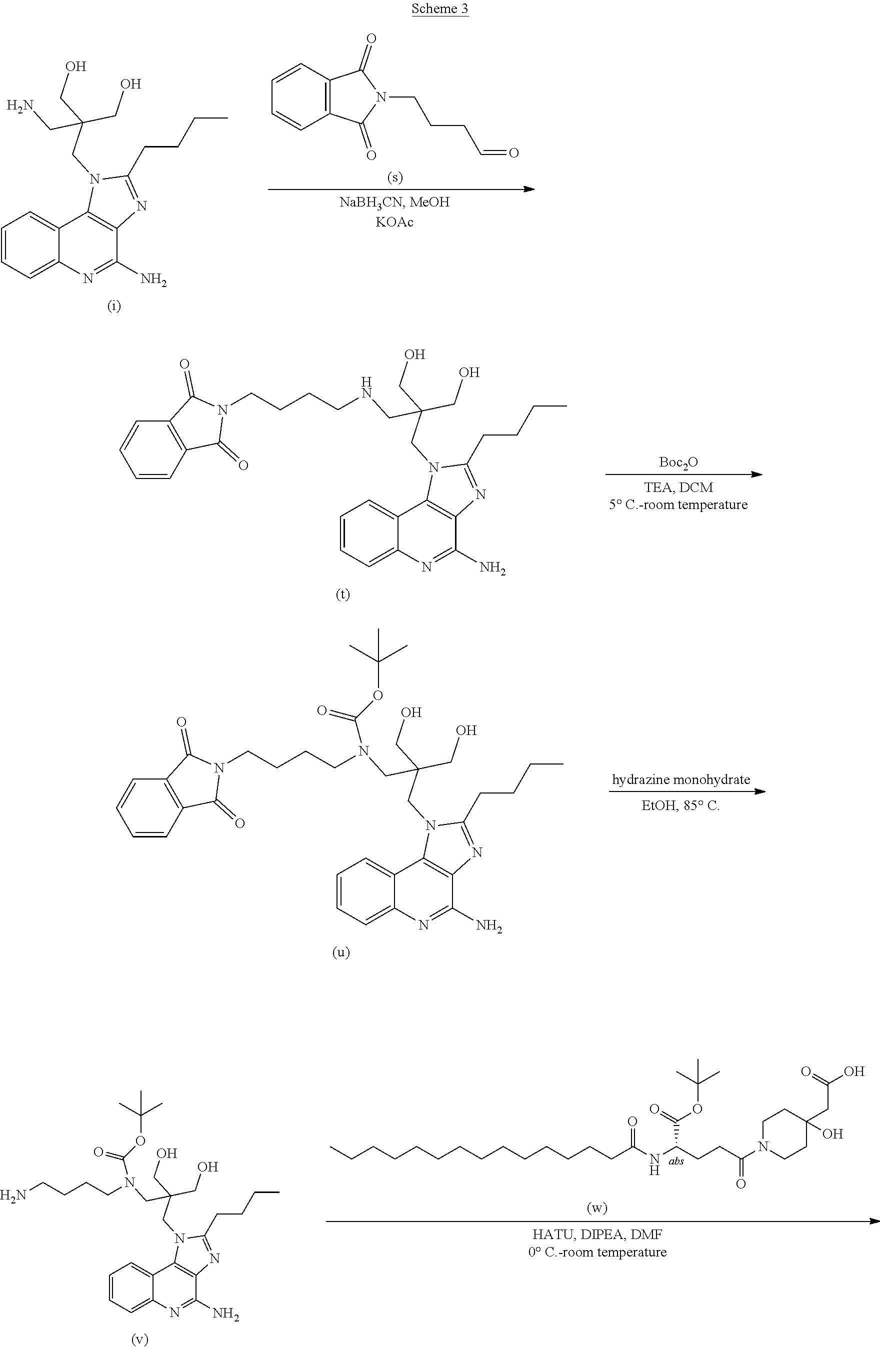

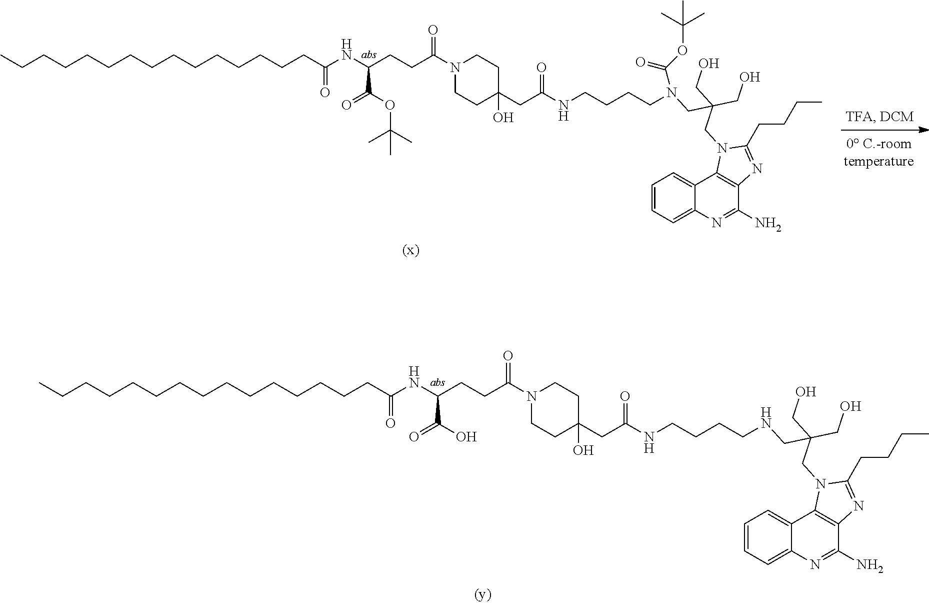

The amine (i) can undergo a reductive amination with the aldehyde(s) in a solvent like MeOH with a mild reducing agent such as sodium cyanoborohydride (NaBH3CN) and with a weak acid like potassium acetate (KOAc) stirred from 16-24 hours to afford the protected intermediate (t). The secondary amine (t) can be protected with Boc2O with a base like TEA in a solvent like DCM from 5° C. to room temperature with stirring from 16-24 hours to provide the protected amine (u). The NHS-ester intermediate (u) can be deprotected with hydrazine monohydrate in a solvent like ethanol (EtOH) with heat from 80-100° C. with stirring from 16-24 hours to form the terminal amine (v). The terminal amine (v) can react with the acid (w) through standard amide coupling conditions like 1-[bis(dimethylamino)methylene]-1H-1,2,3-triazolo[4,5-b]pyridinium 3-oxid hexafluorophosphate (HATU) with a base like N,N-diisopropylethylamine (DIPEA) in a solvent like DMF from 0° C. to room temperature with stirring from 16-24 hours to form the amide (x). The protected acid (x) can be deprotected under standard acidic conditions like TFA in a solvent like DCM from 0° C. to room temperature to provide the target product (y).

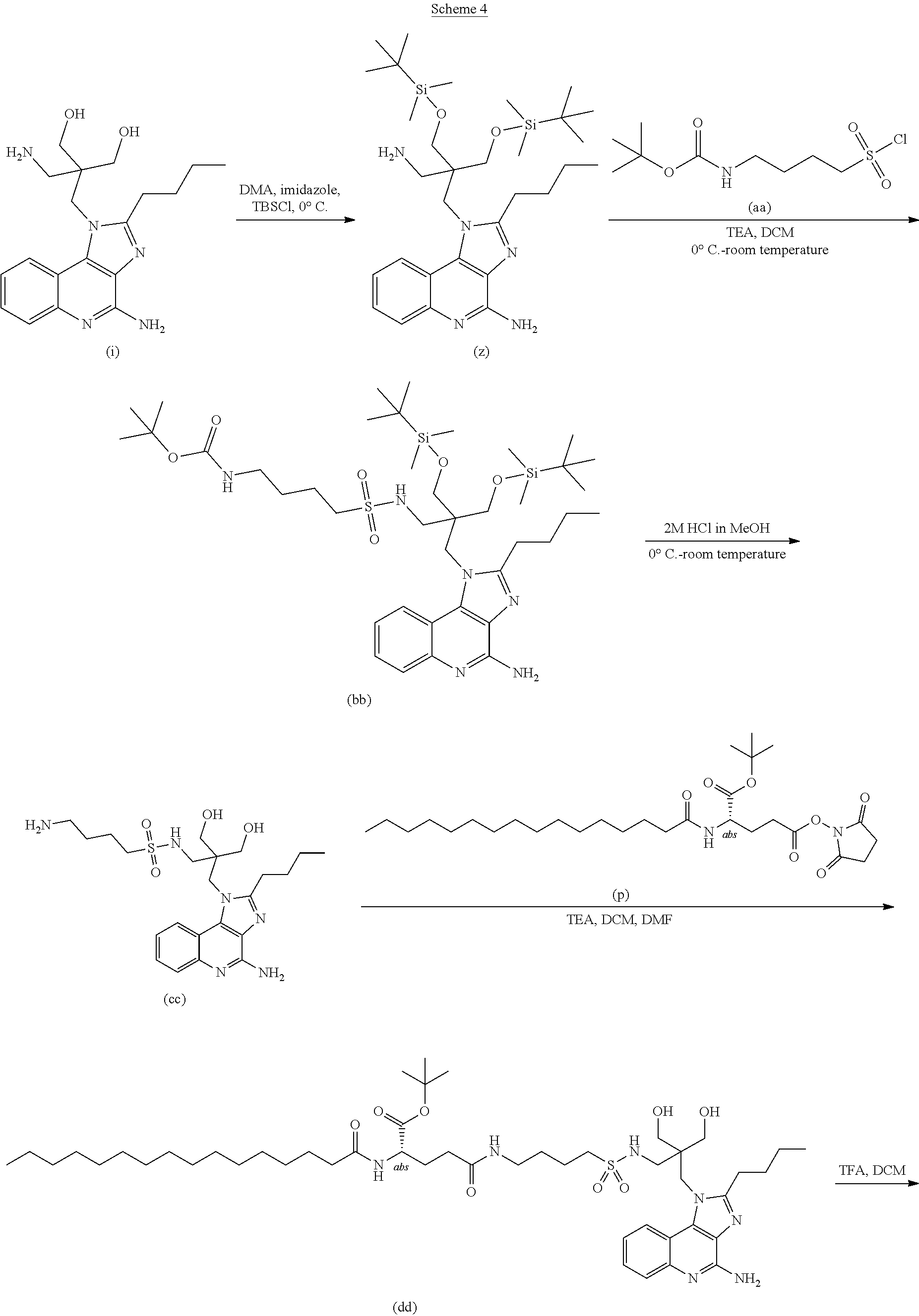

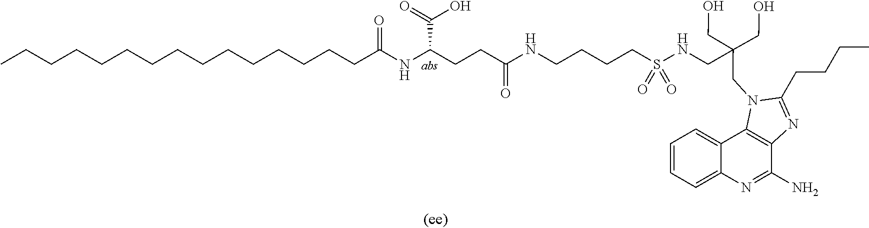 [SMILES: CCCCCCCCCCCCCCCC(=O)N[C@@H](CCC(=O)NCCCCS(=O)(=O)NCC(CO)(CO)Cn1c(CCCC)nc2c(N)nc3ccccc3c21)C(=O)O]

The alcohol intermediate (i) can be protected by reacting with tert-butyldimethylsilyl chloride (TBSCl) with a base like imidazole in a solvent like dimethylacetamide (DMA) from 0° C. to room temperature to provide the protected alcohol (z). The amine (z) can react with sulfonyl chloride (aa) with a base like TEA in a solvent like DCM from 0° C. to room temperature to afford the protected terminal amine (bb) which can be deprotected under acidic conditions such as with HCl in a solvent like MeOH from 0° C. to room temperature to form the amine (cc). The amine (cc) can react with the NHS-ester (p) in solvents like DCM and DMF with a base like TEA to afford the protected acid intermediate (dd) which can be deprotected under standard acidic conditions like TFA in a solvent like DCM to provide the target product (ee).

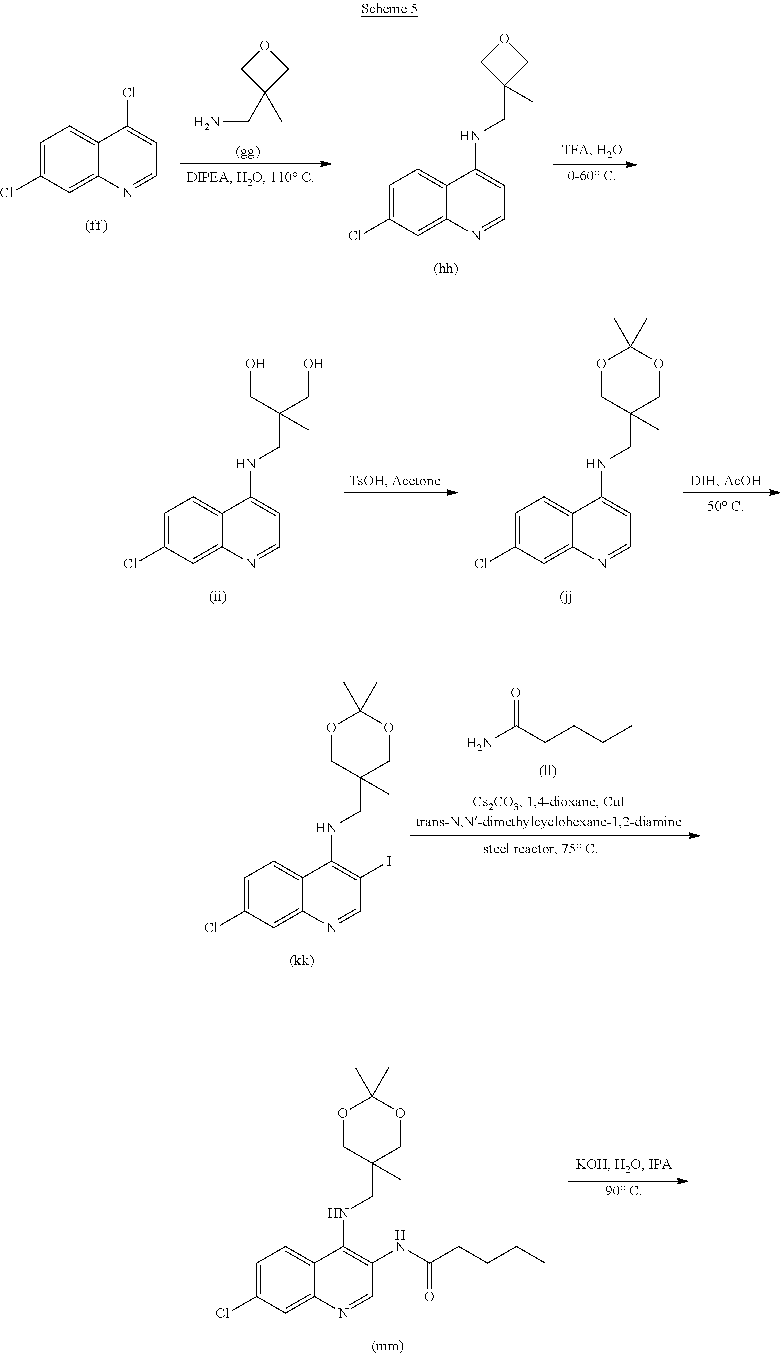

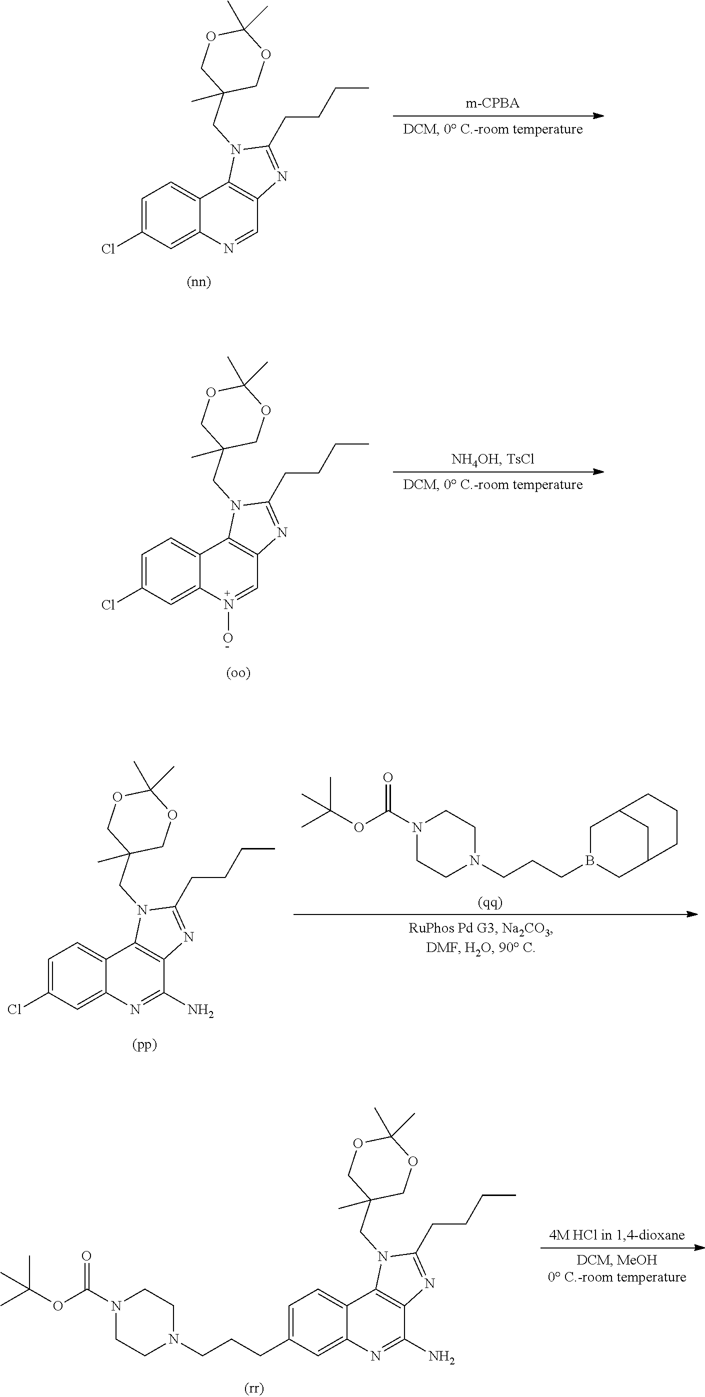

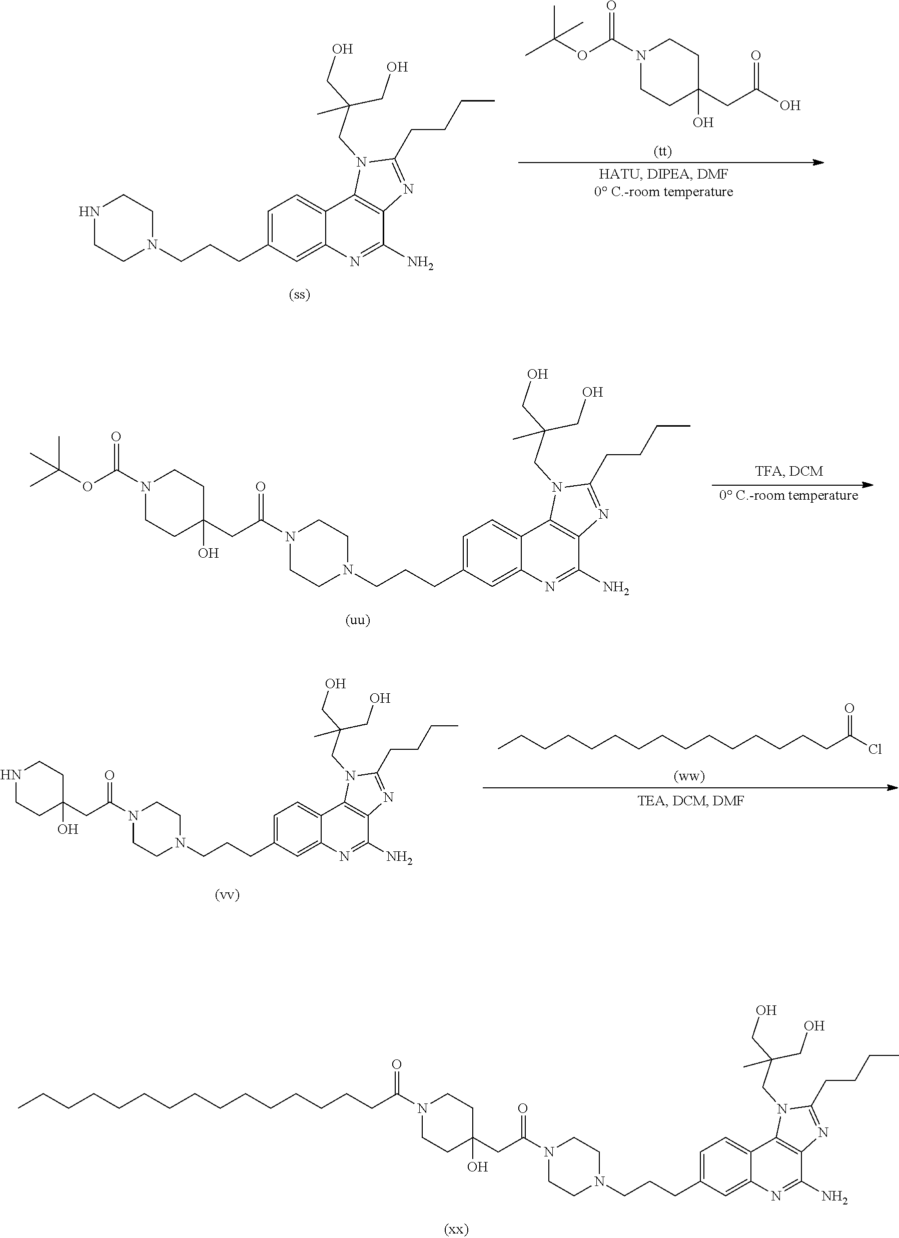

The halogen (e.g. CI)-substituted heterocycle (ff) can react with the amine (gg) in solvents such as DMA and water (H₂O) with a base like DIPEA succumb to heat from 90-110° C. with stirring from 24-72 hours to provide the protected amine (hh). The protected amine (hh) can be deprotected under standard acidic conditions like TFA in a solvent like H₂O with heat from 50-80° C. to provide the alcohol (ii). The alcohol (ii) in a solvent like acetone can be protected by reacting with p-toluenesulfonic acid (TsOH) then stirring for 16-24 hours to afford the heterocycle (jj). The heterocycle (jj) can undergo an iodination by reacting with 1,3-diiodo-5,5-dimethylhydantoin (DIH) in an acid like AcOH with heat from 30-50° C. to form the iodo-substituted heterocycle (kk). The iodo-substituted heterocycle (kk) can react with the pentanamide (II) in a solvent like 1,4-dioxane, a base like cesium carbonate (Cs₂CO₃), a catalyst such as copper (I) iodide (CuI) and a ligand like trans-N,N′-dimethylcyclohexane-1,2-diamine with heat from 50-80° C. then stirring between 16-24 hours to form the amide intermediate (mm). The amide intermediate (mm) can be cyclized with a base like potassium hydroxide (KOH) in suitable solvents such as H₂O and IPA with heat from 90-110° C. and with stirring for 16-24 hours to afford the tricycle (nn). The tricycle (nn) in a solvent such as DCM can oxidize with the treatment of m-CPBA from 0° C. to room temperature with stirring from 16-24 hours to provide the N-oxide (oo). The N-oxide (oo) in a solvent like DCM with a base such as NH₄OH can react with TsCI from 0° C. to room temperature from 16-24 hours to afford the aniline (pp). The chloro-substituted heterocycle (pp) can react with the 9-BBN (9-borabicyclo[3.3.1]nonane) intermediate (qq) with a catalyst like RuPhos Pd G3 and a base like sodium carbonate (Na2CO3) in solvents like DMF and H₂O with heat from 80-100° C. then with stirring for 16-24 hours to afford the protected amine (rr). The protected amine (rr) can be deprotected under standard acidic conditions like HCl in 1,4-dioxane in solvents like DCM and

MeOH from 0° C.-room temperature with stirring from 16-24 hours to provide the secondary amine (ss). The secondary amine (ss) can react with the acid (tt) under standard amide coupling conditions like HATU with a base like DIPEA in a solvent like DMF from 0° C.-room temperature with stirring from 16-24 hours to form the amide (uu). The protected secondary amine (uu) can be deprotected under standard acidic conditions like TFA in a solvent like DCM from 0° C.-room temperature to provide the secondary amine (vv) which can react with the acid chloride (ww) with a base like TEA in solvents like DCM and DMF to provide the target product (xx).

[chemical structure]

The halogen (e.g. CI, Br)-substituted heterocycle (yy) can react with the amine (zz) in a solvent such as DCM and with a base such as TEA from 10° C. to room temperature to afford the nitro-intermediate (aA). The nitro-intermediate (aA) can then be reduced through a hydrogenation with a catalyst such as platinum on carbon (Pt/C) under H₂ gas in a solvent like THF with stirring from 24-48 hours to afford the aniline (bB). The aniline (bB) can react with acid chloride (cC) in a solvent like DCM at 0° C.-room temperature to afford the amide (dD). The amide (dD) can cyclize under basic conditions like sodium hydroxide (NaOH) in a suitable solvent such as EtOH heated from 80-110° C. to provide the tricycle (eE). The tricycle (eE) in a solvent such as DCM can oxidize with the treatment of m-CPBA then with stirring from 16-24 hours to provide the N-oxide (fF). The N-oxide (fF) in a solvent like DCM with a base such as NH₄OH can react with TsCI from 0° C. to room temperature with stirring from 16-24 hours to afford the aniline (gG). The Br-substituted tricycle (gG) can react with the acetylene (hH) under standard cross-coupling conditions like with a catalyst such as tetrakis (triphenylphosphine) palladium (0)(Pd (PPh3) 4) and CuI with a base like Cs2CO3 in a solvent like DMF under argon gas while heating from 80-100° C. with stirring from 16-24 hours to afford the acetylene intermediate (il). The acetylene intermediate (il) can be reduced through standard hydrogenation conditions like with a catalyst such as palladium on carbon (Pd/C) under H₂ gas in a solvent like MeOH with stirring from 16-24 hours to afford the protected amine (jJ) which can undergo deprotection under acidic conditions such as with HCl in EtOAc with additional solvents like DCM and MeOH to form the secondary amine (kK). The secondary amine (KK) can react with the acid (IL) under standard amide coupling conditions like HATU with a base like TEA in a solvent like DMF while stirring from 16-24 hours to form the protected amine (mM) which can then be deprotected under standard acidic conditions like TFA in a solvent like DCM from 0° C.-room temperature to provide the amine (nN). The amine (nN) can react with the NHS-ester (p) in a solvents like DCM and DMF with a base like TEA and stirring from 16-24 hours to afford the protected acid intermediate (oO) which can be deprotected under standard acidic conditions like TFA in a solvent like DCM from 0° C. to room temperature to provide the target product (pP).

### EXAMPLES

In order that this disclosure may be better understood, the following examples are set forth. These examples are for purposes of illustration only and are not to be construed as limiting the scope of the disclosure in any manner.

Unless noted otherwise (below or in the schemes or preparations above), all reactants were obtained commercially without further purifications or were prepared using methods known in the literature.

### Example 1

### N5-(4-(N-(3-(4-Amino-2-butyl-1H-imidazo[4,5-c]quinolin-1-yl)-2,2-bis(hydroxymethyl)propyl)sulfamoyl)butyl)-N2-palmitoyl-L-glutamine (1)

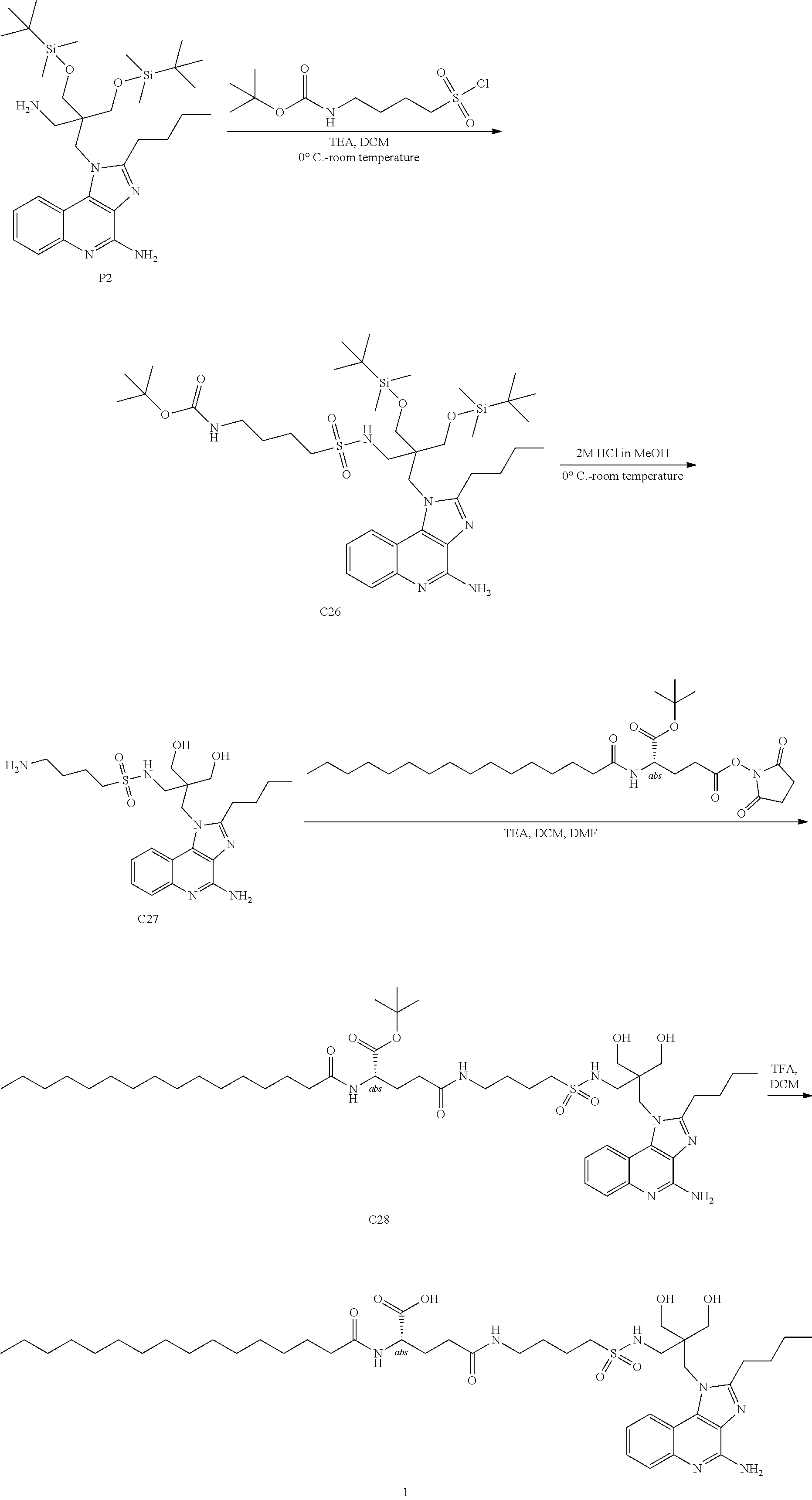

Step 1. Preparation of tert-butyl (4-(N-(3-(4-amino-2-butyl-1H-imidazo[4,5-c]quinolin-1-yl)-2.2-bis(((tert-butyldimethylsilyl)oxy)methyl)propyl)sulfamoyl)butyl) carbamate (C26)

At 0° C. under N₂ gas, to a stirred solution of P2 (14 g, 24 mmol) in DCM (140 mL) was added TEA (9.5 g, 94 mmol) then tert-butyl (4-(chlorosulfonyl)butyl) carbamate (CAS: 2167808-49-1; 7.0 g, 26 mmol) successively. The reaction mixture was warmed to room temperature then stirred for 2 hours before H₂O (100 mL) was added. The diluted reaction mixture was extracted with DCM (200 mL×2) then the combined organic layer was washed with brine (50 ml×2), dried over Na₂SO₄ and concentrated in vacuo. The yellow gum was purified by column chromatography (silica gel, MeOH: DCM, 0-15% MeOH) to provide an impure batch of C26 (16 g) as a yellow solid. The impure batch of C26 (16 g) was repurified by column chromatography (silica gel, MeOH: EtOAc, 0-15% MeOH) to provide C26 (13 g, 67% yield) as a light-yellow solid. (LCMS)(M)⁺ 821.5.

Step 2. Preparation of 4-amino-N-(3-(4-amino-2-butyl-1H-imidazo[4,5-c]quinolin-1-yl)-2.2-bis(hydroxymethyl)propyl) butane-1-sulfonamide hydrochloride (C27)

At 0° C., to a stirred solution of C26 (36.7 g, 44.7 mmol) in MeOH (50 mL) was added 2M HCl in MeOH (500 mL). The reaction mixture was warmed to room temperature and stirred for 3 hours then concentrated in vacuo to give a yellow gum. The yellow gum was lyophilized to provide C27 (50.4 g, >99% yield) as a light-yellow solid. The solid was used directly in the next step without any additional purification. (LCMS)(M+H)⁺ 493.2. ¹H NMR (400 MHz, (CD₃)₂SO) δ 14.11 (br s, 1H), 8.86-8.53 (m, 2H), 8.07 (br s, 2H), 7.81 (d, 1H), 7.70 (t, 1H), 7.49 (t, 1H), 7.43 (t, 1H), 4.97-4.87 (m, 1H), 4.66-4.53 (m, 1H), 3.52-3.32 (m, 3H), 3.21-2.89 (m, 9H), 2.81-2.71 (m, 2H), 1.87-1.61 (m, 6H), 1.49-1.37 (m, 2H), 0.94 (t, 3H).

Step 3. Preparation of tert-butyl N⁵-(4-(N-(3-(4-amino-2-butyl-1H-imidazo[4,5-c]quinolin-1-yl)-2,2-bis(hydroxymethyl)propyl)sulfamoyl)butyl)-N²-palmitoyl-L-glutaminate (C28)

Under N₂ gas, to a stirred solution of C27 (2.52 g, 4.46 mmol) in DCM (30 mL) and DMF (10 mL) was added TEA (1.35 g, 13.4 mmol) and 1-tert-butyl 5-(N-succinimidyl)N-palmitoyl-L-glutamate (CAS: 204521-63-1; 2.40 g, 4.45 mmol). The reaction mixture was stirred at room temperature for 2 hours before H₂O (50 mL) was added and then extracted with DCM (50 mL×2). The combined organic layer was washed with brine (50 mL), dried with Na₂SO₄, filtered, and concentrated in vacuo. The light-yellow gum was purified by column chromatography (silica gel, MeOH: DCM, 0-20% MeOH) to provide C28 (3.50 g, 85.7% yield) as a light-yellow solid. (LCMS)(M)⁺ 916.8. ¹H NMR (400 MHz, (CD₃)₂SO) δ 8.64 (d, 1H), 8.07 (d, 1H), 7.85 (t, 1H), 7.66 (d, 1H), 7.33-7.15 (m, 3H), 7.24 (br s, 1H), 7.09 (t, 1H), 4.99-4.84 (m, 2H), 4.64-4.47 (m, 2H), 4.14-3.98 (m, 1H), 3.57-3.48 (m, 1H), 3.18-3.07 (m, 3H), 3.03-2.90 (m, 6H), 2.17-2.06 (dt, 4H), 1.94-1.84 (m, 1H), 1.82-1.69 (m, 3H), 1.63-1.52 (m, 2H), 1.51-1.40 (m, 6H), 1.39 (br s, 9H), 1.22 (br s, 26H), 0.94 (t, 3H), 0.84 (t, 3H).

Step 4. Preparation of N⁵-(4-(N-(3-(4-Amino-2-butyl-1H-imidazo[4,5-c]quinolin-1-yl)-2.2-bis(hydroxymethyl)propyl)sulfamoyl)butyl)-N²-palmitoyl-L-glutamine (1)

To a stirred solution of C28 (3.50 g, 3.82 mmol) in DCM (15 mL) was added TFA (15 mL) dropwise at room temperature then stirred for 2 hours. The reaction mixture was dissolved in (1:2) H2O: THF (30 mL) then LiOH (962 mg, 22.9 mmol) was added to adjust the pH˜8. The reaction was stirred for 1 hour at room temperature then purified by reverse phase HPLC (Welch Xtimate C₁₈ 150 mm×25 mm×5 μm, water (0.05% NH₄HCO₃)/MeCN, 35 to 75% over 11 minutes, 100% MeCN hold time for 3 minutes, flow rate was 60 (mL/min)) and lyophilized to provide 1 (1.75 g, 53.3% yield) as a white solid. LCMS m/z (M)⁺=860.4. ¹H NMR (400 MHz, (CD₃)₂SO) δ 8.60 (d, 1H), 7.95-7.84 (m, 1H), 7.82-7.74 (m, 1H), 7.65 (d, 1H), 7.45 (t, 2H), 7.26 (t, 2H), 7.07-6.97 (m, 1H), 4.95-4.84 (m, 1H), 4.60-4.48 (m, 1H), 4.16-4.07 (m, 1H), 3.60-3.30 (m, 4H), 3.22-3.08 (m, 2H), 3.03-2.66 (m, 8H), 2.16-2.06 (m, 4H), 2.01-1.90 (m, 1H), 1.83-1.70 (m, 3H), 1.54-1.33 (m, 8H), 1.21 (br s, 24H), 0.94 (t, 3H), 0.84 (t, 3H).

### Example 2

### (S)-1-(N-(3-(4-Amino-2-butyl-1H-imidazo[4,5-c]quinolin-1-yl)-2,2-bis(hydroxymethyl)propyl)sulfamoyl)-13-oxo-16-palmitamido-3,6,9-trioxa-12-azaheptadecan-17-oic acid (2)

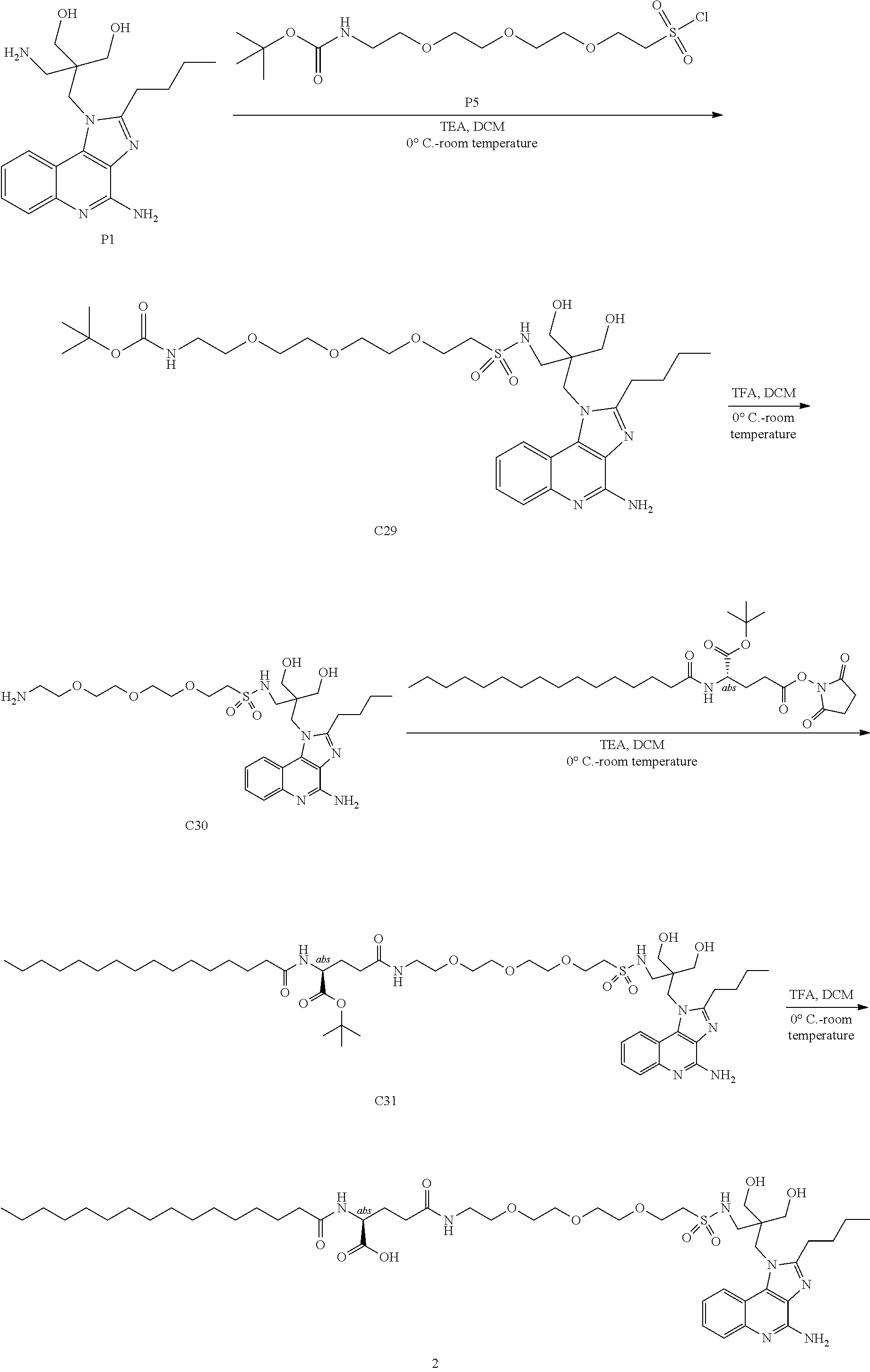

Step 1. Preparation of tert-butyl (2-(2-(2-(2-(N-(3-(4-amino-2-butyl-1H-imidazo[4,5-c]quinolin-1-yl)-2.2-bis(hydroxymethyl)propyl)sulfamoyl)ethoxy)ethoxy)ethoxy)ethyl)carbamate (C29)

The reaction was conducted in two batches then combined for purification. At 0-5° C. under N₂ gas, to a stirred solution of P1 (91.6 mg, 0.213 mmol) in DCM (10 mL) was added TEA (64.6 mg, 0.639 mmol) and P5 (0.160 g, 0.426 mmol) to form the first batch. The reaction mixture of the first batch was warmed to room temperature then stirred for 16 hours.

A second batch of the same reaction was conducted with P1 (22.9 mg, 0.0532 mmol). The 2 batches were combined then an additional portion of TEA (64.6 mg, 0.639 mmol) and P5 (0.120 g, 0.319 mmol) were added. The combined reaction mixture was stirred at room temperature for 16 hours was extracted with EtOAc (20 mL×3). The combined organic layer was washed with brine (20 mL×3), dried with Na₂SO₄, filtered, and concentrated in vacuo to provide C29 (95.0 mg, 51.2% yield) as a yellow gum. The gum was used directly in the next step without further purification. LCMS m/z (M+H)⁺=697.4.

Step 2. Preparation of N-(3-(4-amino-2-butyl-1H-imidazo[4,5-c]quinolin-1-yl)-2,2-bis(hydroxymethyl)propyl)-2-(2-(2-(2-aminoethoxy) ethoxy) ethoxy) ethane-1-sulfonamide trifluoroacetate (C30)

At 0-5° C. under N₂ gas, to a stirred solution of C29 (95.0 mg, 0.14 mmol) in DCM (2 mL) was added TFA (1 mL) dropwise. The reaction mixture was stirred at room temperature for 1 hour then concentrated in vacuo and lyophilized to provide C30 (0.100 g, >99% yield) as a yellow gum. The gum was used directly in the next step without further purification. LCMS m/z (M+H)⁺=597.4.

Step 3. Preparation of tert-butyl(S)-1-(N-(3-(4-amino-2-butyl-1H-imidazo[4,5-c]quinolin-1-yl)-2.2-bis(hydroxymethyl)propyl)sulfamoyl)-13-oxo-16-palmitamido-3,6,9-trioxa-12-azaheptadecan-17-oate (C31)

At 0-5° C. under N₂ gas, to a stirred solution of C30 (0.100 g, 0.168 mmol) and 1-(tert-butyl) 5-(2,5-dioxopyrrolidin-1-yl) palmitoyl-L-glutamate (CAS: 204521-63-1; 90.3 mg, 0.168 mmol) in DCM (5 mL) was added TEA (50.9 mg, 0.503 mmol). The reaction mixture was stirred at room temperature for 2 hours then was extracted with the DCM (15 mL×3). The combined organic layer was washed with brine (20 mL×3), dried with Na₂SO₄, filtered, and concentrated in vacuo to provide C31 (0.180 g, >99% yield) as yellow gum. The gum was used directly in the next step without further purification.

Step 4. Preparation of(S)-1-(N-(3-(4-amino-2-butyl-1H-imidazo[4,5-c]quinolin-1-yl)-2.2-bis(hydroxymethyl)propyl)sulfamoyl)-13-oxo-16-palmitamido-3,6,9-trioxa-12-azaheptadecan-17-oic acid (2)

At 0-5° C. under N₂ gas, to a stirred solution of C31 (0.180 g, 0.176 mmol) in DCM (5 mL) was added TFA (2 mL) dropwise. The reaction was warmed to room temperature and stirred for 3 hours then concentrated in vacuo. The yellow gum was purified by reverse phase HPLC (C18 150 mm×30 mm×5 μm, water (0.05% formic acid)/MeCN, 41 to 81% over 9 minutes, 100% MeCN hold time for 2 minutes, flow rate was 30 (mL/min)) and lyophilized to provide 2 (14.3 mg, 8.45% yield) as a white solid. LCMS m/z (M+H)⁺=965.7. ¹H NMR (400 MHz, (CD₃)₂SO) δ 8.62 (d, 1H), 8.16 (s, 1H), 7.95-7.94 (m, 1H), 7.65 (d, 1H), 7.47 (t, 1H), 7.28 (t, 1H), 4.95-4.85 (m, 1H), 4.60-4.50 (m, 1H), 4.15-4.07 (m, 1H), 3.49 (s, 14H), 3.38 (t, 4H), 3.22-3.09 (m, 4H), 3.02-2.88 (m, 2H), 2.15-2.06 (m, 4H), 1.99-1.89 (m, 1H), 1.82-1.70 (m, 3H), 1.49-1.37 (m, 4H), 1.21 (d, 26H), 0.93 (t, 3H), 0.83 (t, 3H).

### Example 3

### (S)-1-(4-Amino-2-butyl-1H-imidazo[4,5-c]quinolin-1-yl)-2,2-bis(hydroxymethyl)-18-oxo-21-palmitamido-8, 11, 14-trioxa-4, 17-diazadocosan-22-oic acid (3)

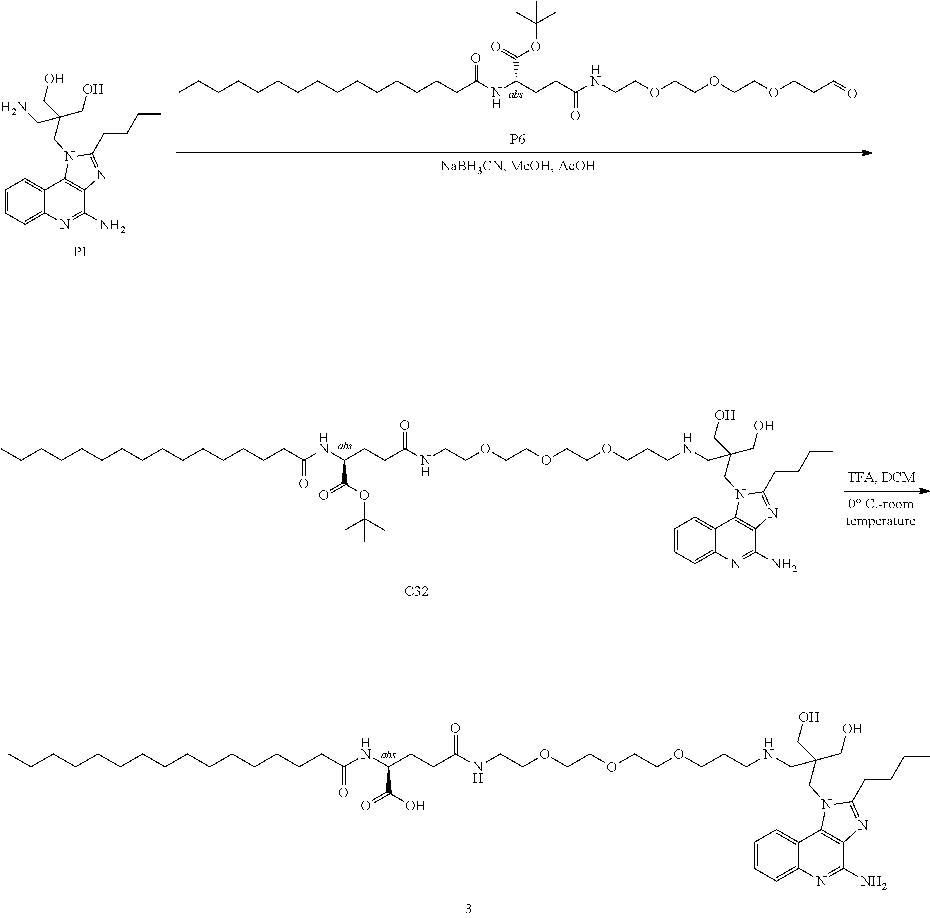

Step 1. Preparation of tert-butyl(S)-1-(4-amino-2-butyl-1H-imidazo[4,5-c]quinolin-1-yl)-2,2-bis(hydroxymethyl)-18-oxo-21-palmitamido-8, 11, 14-trioxa-4, 17-diazadocosan-22-oate (C32)

To a stirred solution of P6 (93.8 mg, 0.119 mmol), 4 Å molecular sieves (CAS: 70955-01-0; 60.0 mg) and P1 (64.2 mg, 0.119 mmol) in MeOH (2.0 mL) was added NaBH3CN (37.5 mg, 0.596 mmol) which caused bubbles to form. The reaction mixture was stirred at room temperature for 2 hours before AcOH (0.1 mL) was added dropwise. After the addition, the reaction mixture was stirred for an additional 2 hours before another portion of P6 (0.107 g, 0.170 mmol) was added. The suspension was stirred for 15 minutes at room temperature before another portion of NaBH3CN (30.0 mg, 0.477 mmol) was added. The reaction mixture was stirred for 2 hours at room temperature then poured into H₂O (15.0 mL) and NaHCO₃ (10.0 mL) was added. The suspension was extracted with DCM (15.0 mL×3). The combined organic layer was dried over Na₂SO₄, filtered, and concentrated in vacuo. The yellow gum was purified by prep-TLC ((10:1:0.1) DCM: MeOH: NH₄OH, Rf˜0.3, UV 254 nm) to provide C32 (50.0 mg, 43.3% yield) as a colorless solid. LCMS m/z (M)⁺=970.7.

Step 2. Preparation of(S)-1-(4-amino-2-butyl-1H-imidazo[4,5-c]quinolin-1-yl)-2,2-bis(hydroxymethyl)-18-oxo-21-palmitamido-8, 11,14-trioxa-4, 17-diazadocosan-22-oic acid (3)

At 0-5° C. under N₂ gas, to a stirred solution of C32 (50.0 mg, 0.0515 mmol) in DCM (3 mL) was added TFA (1 mL) dropwise. The reaction mixture was warmed to room temperature and stirred for 2 hours then concentrated in vacuo. The yellow gum was purified by reverse phase HPLC (Boston Prime C₁₈ 150 mm×30 mm×5 μm, water (0.05% NH₄OH)/MeCN, 60 to 80% over 11 minutes, 100% MeCN hold time for 2 minutes, flow rate was 35 (mL/min)) to provide 3 (14.5 mg, 30.7% yield) as a white solid. LCMS m/z (M)⁺=914.6. ¹H NMR (400 MHz, CDCl₃) δ 8.42-8.34 (m, 1H), 7.79-7.72 (m, 1H), 7.68-7.61 (m, 1H), 7.51-7.43 (m, 1H), 7.38 (t, 1H), 6.79-6.70 (m, 1H), 4.97-4.84 (m, 1H), 4.56-4.41 (m, 1H), 4.34-4.24 (m, 1H), 3.99-3.89 (m, 1H), 3.80-3.67 (m, 1H), 3.52 (s, 13H), 3.44-3.29 (m, 3H), 3.20-3.00 (m, 3H), 2.89-2.69 (m, 3H), 2.39-2.11 (m, 6H), 2.03-1.92 (m, 1H), 1.85-1.76 (m, 3H), 1.67-1.59 (m, 2H), 1.50-1.40 (m, 2H), 1.32-1.22 (m, 26H), 0.96 (t, 3H), 0.87 (t, 3H).

### Example 4

### (S)-5-(4-(2-((4-((3-(4-Amino-2-butyl-1H-imidazo[4,5-c]quinolin-1-yl)-2,2-bis(hydroxymethyl)propyl)amino)butyl) amino)-2-oxoethyl)-4-hydroxypiperidin-1-yl)-5-oxo-2-palmitamidopentanoic acid (4)

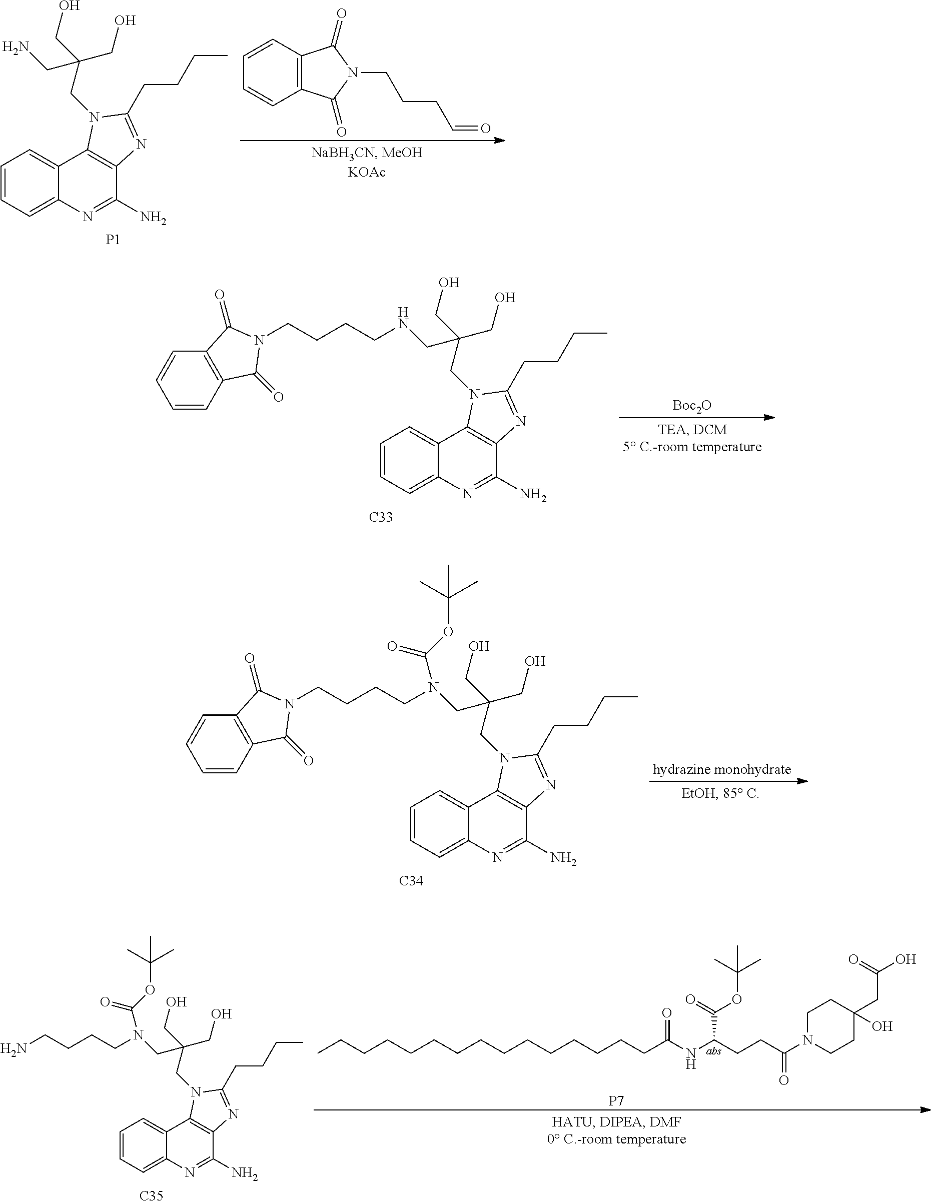

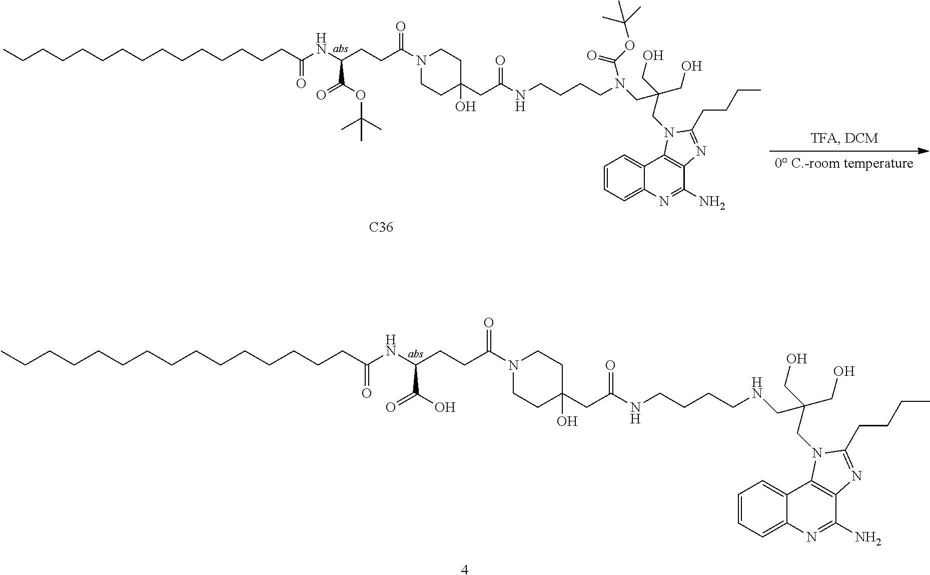

Step 1. Preparation of 2-(4-((3-(4-amino-2-butyl-1H-imidazo[4,5-c]quinolin-1-yl)-2.2-bis(hydroxymethyl)propyl)amino)butyl) isoindoline-1,3-dione (C33)

A solution of P1 (0.200 g, 0.465 mmol), 4-(1,3-dioxoisoindolin-2-yl) butanal (CAS: 3598-60-5; 0.106 g, 0.486 mmol) and KOAc (95.4 mg, 0.972 mmol) in MeOH (8 mL) was stirred for 30 minutes at room temperature. To the reaction mixture was added NaBH3CN (41.7 mg, 0.663 mmol). The suspension was stirred at room temperature for 18 hours before the reaction mixture was quenched with ice (3 mL). The quenched reaction mixture was adjusted to pH=9 with the aqueous Na₂CO3 then extracted with (4:1) CHCl₃: IPA (10 mL×5). The combined organic layer was dried over Na₂SO₄ and concentrated in vacuo to provide C33 (260 mg, >99% yield) as yellow gum. The gum was used directly in the next step without additional purification. LCMS m/z (M+H)⁺=559.4.

Step 2. Preparation of tert-butyl (3-(4-amino-2-butyl-1H-imidazo[4,5-c]quinolin-1-yl)-2,2-bis(hydroxymethyl)propyl)(4-(1,3-dioxoisoindolin-2-yl)butyl) carbamate (C34)

At 5-10° C., to a solution of C33 (0.260 g, 0.465 mmol) and TEA (0.141 g, 1.40 mmol) in DCM (8 mL) was added Boc2O (0.152 g, 0.698 mmol). The reaction mixture was warmed to room temperature and stirred for 18 hours then poured into H₂O (10 mL). The quenched reaction mixture was extracted with DCM (10 mL×3). The combined organic layer was dried with Na₂SO₄ and concentrated in vacuo. The brown gum was purified by column chromatography (silica gel, MeOH: DCM, 0-10% MeOH) to provide C34 (166 mg, 54.1% yield) as yellow gum. LCMS m/z (M+H)⁺=659.4. ¹H NMR (400 MHz, (CD₃)₂SO) δ 8.41-8.31 (m, 1H), 7.92-7.83 (m, 4H), 7.52 (d, 1H), 7.32-7.24 (m, 1H), 7.18-7.10 (m, 1H), 6.52 (br s, 2H), 5.24 (s, 1H), 4.91-4.69 (m, 2H), 4.65-4.51 (m, 1H), 3.57-3.47 (m, 2H), 3.46-3.36 (m, 4H), 3.25-3.05 (m, 4H), 3.06-2.78 (m, 2H), 1.85-1.65 (m, 2H), 1.51-1.34 (m, 6H), 1.28 (br s, 9H), 0.94 (t, 3H).

Step 3. Preparation of tert-butyl (3-(4-amino-2-butyl-1H-imidazo[4,5-c]quinolin-1-yl)-2,2-bis(hydroxymethyl)propyl)(4-aminobutyl) carbamate (C35)

Under N₂ gas, to a stirred solution of C34 (0.166 g, 0.252 mmol) in EtOH (8 mL) was added hydrazine monohydrate (0.260 g, 5.19 mmol) at room temperature. The reaction mixture was heated to 85° C. and stirred for 16 hours before filtration. The filter cake was washed with EtOH (15 mL) then the filtrate was concentrated in vacuo. The residue was suspended in DCM (20 mL) and filtered again. The filtrate was dried with Na₂SO4 then concentrated in vacuo and lyophilized to provide C35 (131 mg, 98.3% yield) as white solid. The solid was used directly in the next step without further purification. LCMS m/z (M+H)⁺=529.5.

Step 4. Preparation of tert-butyl(S)-5-(4-(2-((4-((3-(4-amino-2-butyl-1H-imidazo[4,5-c]quinolin-1-yl)-2.2-bis(hydroxymethyl)propyl)(tert-butoxycarbonyl)amino)butyl)amino)-2-oxoethyl)-4-hydroxypiperidin-1-yl)-5-oxo-2-palmitamidopentanoate (C36)

At 0° C. under N₂ gas, to a stirred solution of C35 (0.131 g, 0.248 mmol) and P7 (0.144 g, 0.248 mmol) in DMF (4 mL) was added HATU (98.9 mg, 0.260 mmol) then DIPEA (0.192 g, 1.49 mmol) dropwise. The reaction mixture was stirred for 16 hours at room temperature then poured into H₂O (10 mL). The diluted reaction mixture was extracted with EtOAc (10 mL×3) then the combined organic layer was dried with Na₂SO4 and concentrated in vacuo. The yellow gum was purified by column chromatography (silica gel, ((1:10) NH₄OH: MeOH) in DCM, 0-10% ((1:10) NH₄OH: MeOH)) to provide C36 (205 mg, 74.7% yield) as white solid.

Step 5. Preparation of(S)-5-(4-(2-((4-((3-(4-amino-2-butyl-1H-imidazo[4,5-c]quinolin-1-yl)-2,2-bis(hydroxymethyl)propyl)amino)butyl) amino)-2-oxoethyl)-4-hydroxypiperidin-1-yl)-5-oxo-2-palmitamidopentanoic acid (4)

At 0-5° C. under N₂ gas, to a stirred solution of C36 (0.205 g, 0.185 mmol) in DCM (4 mL) was added TFA (5 mL) dropwise. The reaction mixture was stirred at room temperature for 4 hours then concentrated in vacuo. The yellow gum was purified by reverse phase HPLC (Boston Prime C₁₈ 150 mm×30 mm×5 μm, water (0.05% NH₄OH+NH₄HCO₃)/MeCN, 26 to 56% over 8 minutes, 100% MeCN hold time for 3 minutes, flow rate was 30 (mL/min)) then lyophilized to provide 4 (38.5 mg, 14.9% yield) as a light-yellow glass. LCMS m/z (M+H)⁺=938. 6. ¹H NMR (400 MHz, (CD₃)₂SO) δ 8.70-8.61 (m, 1H), 7.97-7.88 (m, 1H), 7.84-7.74 (m, 1H), 7.63 (d, 1H), 7.42 (t, 1H), 7.23 (d, 1H), 7.15-6.89 (m, 2H), 5.02-4.86 (m, 2H), 4.59-4.47 (m, 1H), 4.17-4.08 (m, 1H), 4.03-3.95 (m, 1H), 3.64-3.23 (m, 14H), 3.14-2.81 (m, 7H), 2.28-2.22 (m, 1H), 2.18 (s, 1H), 2.13-2.06 (m, 2H), 1.93-1.86 (m, 1H), 1.81-1.73 (m, 3H), 1.50-1.31 (m, 11H), 1.22 (s, 24H), 0.94 (t, 3H), 0.84 (t, 3H).

### Example 5

### 1-(4-(2-(4-(3-(4-Amino-2-butyl-1-(3-hydroxy-2-(hydroxymethyl)-2-methylpropyl)-1H-imidazo[4,5-c]quinolin-7-yl)propyl) piperazin-1-yl)-2-oxoethyl)-4-hydroxypiperidin-1-yl) hexadecan-1-one (5)

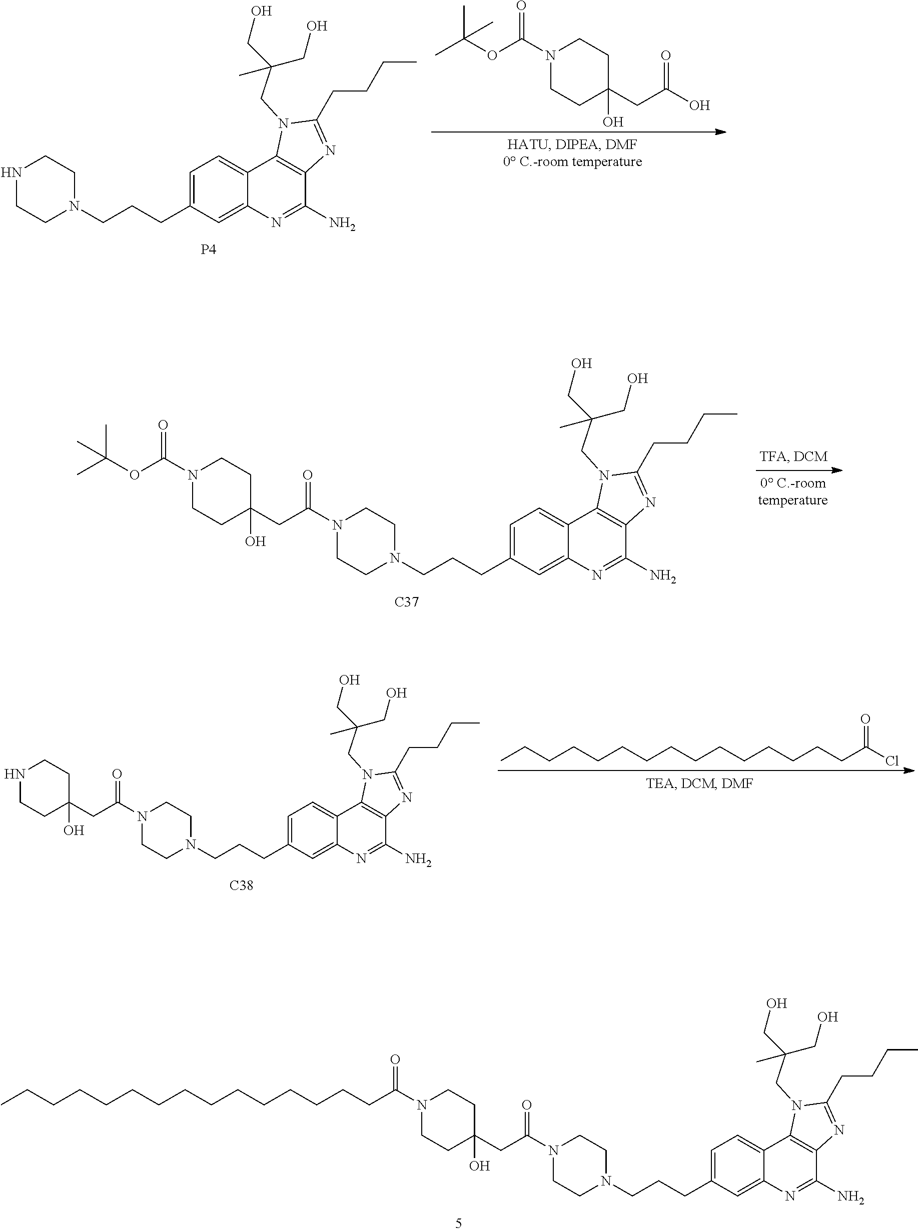

Step 1. Preparation of tert-butyl 4-(2-(4-(3-(4-amino-2-butyl-1-(3-hydroxy-2-(hydroxymethyl)-2-methylpropyl)-1H-imidazo[4,5-c]quinolin-7-yl)propyl) piperazin-1-yl)-2-oxoethyl)-4-hydroxypiperidine-1-carboxylate (C37)

At 0° C. under N₂ gas, to a stirred solution of 2-(1-(tert-butoxycarbonyl)-4-hydroxypiperidin-4-yl) acetic acid (CAS: 502482-52-2; 37.7 mg, 0.145 mmol) in DMF (3 mL) was added HATU (57.9 mg, 0.152 mmol) then DIPEA (89.4 mg, 0.692 mmol) successively. The reaction mixture was stirred for 10 minutes then P4 (80.0 mg, 0.138 mmol) was added. The resulting reaction mixture was stirred at room temperature for 16 hours. The yellow suspension was concentrated in vacuo then filtered through a pad of silica gel. The silica pad was rinsed with EtOAc (˜100 mL) then (1:10) MeOH: DCM. The filtrate was combined then discarded. The silica pad was then rinsed with (10:100:1) MeOH: DCM: NH₄OH (˜200 mL) then the filtrate was concentrated in vacuo to provide C37 (142 mg, >99% yield) as yellow gum. The gum was used directly in the next step without further purification. LCMS m/z (M+H)⁺=710.5. 1H NMR (400 MHz, CDCl₃) δ 8.52 (dd, 1H), 8.34 (d, 1H), 8.19 (d, 1H), 7.79 (s, 1H), 7.24-7.17 (m, 2H), 5.24-5.07 (m, 1H), 4.91-4.57 (m, 1H), 3.81 (br s, 2H), 3.73-3.56 (m, 7H), 3.51-3.45 (m, 2H), 3.26-3.14 (m, 1H), 3.13-3.03 (m, 4H), 2.72 (t, 2H), 2.47-2.30 (m, 8H), 1.87-1.66 (m, 6H), 1.52 (t, 4H), 1.45 (s, 9H), 0.96 (t, 3H), 0.64 (s, 3H).

Step 2. Preparation of 1-(4-(3-(4-amino-2-butyl-1-(3-hydroxy-2-(hydroxymethyl)-2-methylpropyl)-1H-imidazo[4,5-c]quinolin-7-yl)propyl) piperazin-1-yl)-2-(4-hydroxypiperidin-4-yl) ethan-1-one trifluoroacetate (C38)

At 0° C. under N₂ gas, to a stirred solution of C37 (98.0 mg, 0.138 mmol) in DCM (1.5 mL) was added TFA (0.5 mL). The reaction mixture was stirred at room temperature for 1 hour then concentrated in vacuo and lyophilized to provide C38 (0.170 g, >99% yield) as a yellow gum. The gum was used directly in the next step without further purification. LCMS m/z (M+H)⁺=610.8.

Step 3. Preparation of 1-(4-(2-(4-(3-(4-amino-2-butyl-1-(3-hydroxy-2-(hydroxymethyl)-2-methylpropyl)-1H-imidazo[4,5-c]quinolin-7-yl)propyl) piperazin-1-yl)-2-oxoethyl)-4-hydroxypiperidin-1-yl) hexadecan-1-one (5)

At room temperature under N₂ gas, to a stirred solution of C38 (0.133 g, 0.140 mmol) in DMF (1 mL) was added a solution of TEA (113 mg, 1.12 mmol) in DCM (1 mL) dropwise then a solution of palmitoyl chloride (CAS: 112-67-4; 40.3 mg, 0.147 mmol) in DCM (2 mL). The reaction mixture was stirred at room temperature for 1.5 hours then concentrated in vacuo. The light-yellow solid was purified by reverse phase HPLC (Boston Prime C₁₈ 150 mm×30 mm×5 μm, water (0.05% NH₄OH)/(1:1) MeCN: THF, 50 to 80% over 8 minutes, 100% MeCN hold time for 3 minutes, flow rate was 30 (mL/min)) then lyophilized to provide 5 (17.6 mg, 14.9% yield) as a white solid. LCMS m/z (M)⁺=848.6. ¹H NMR (400 MHz, CDCl₃) δ 8.25 (d, 1H), 7.64-7.62 (m, 1H), 7.16 (dd, 1H), 6.16 (br s, 2H), 5.30-5.16 (m, 1H), 4.93-4.77 (m, 1H), 4.71-4.56 (m, 1H), 4.38 (d, 1H), 3.82-3.43 (m, 9H), 3.09-2.99 (m, 3H), 2.78 (t, 2H), 2.48-2.27 (m, 10H), 1.96-1.72 (m, 9H), 1.65-1.56 (m, 2H), 1.52-1.35 (m, 4H), 1.32-1.21 (m, 24H), 0.98 (t, 3H), 0.88 (t, 3H), 0.64 (s, 3H).

### Example 6

### (S)-1-(4-(3-(4-Amino-2-(ethoxymethyl)-1-(3-hydroxy-2-(hydroxymethyl)-2-methylpropyl)-1H-imidazo[4,5-c]quinolin-7-yl)propyl) piperazin-1-yl)-1, 14-dioxo-17-palmitamido-4,7,10-trioxa-13-azaoctadecan-18-oic acid (6)

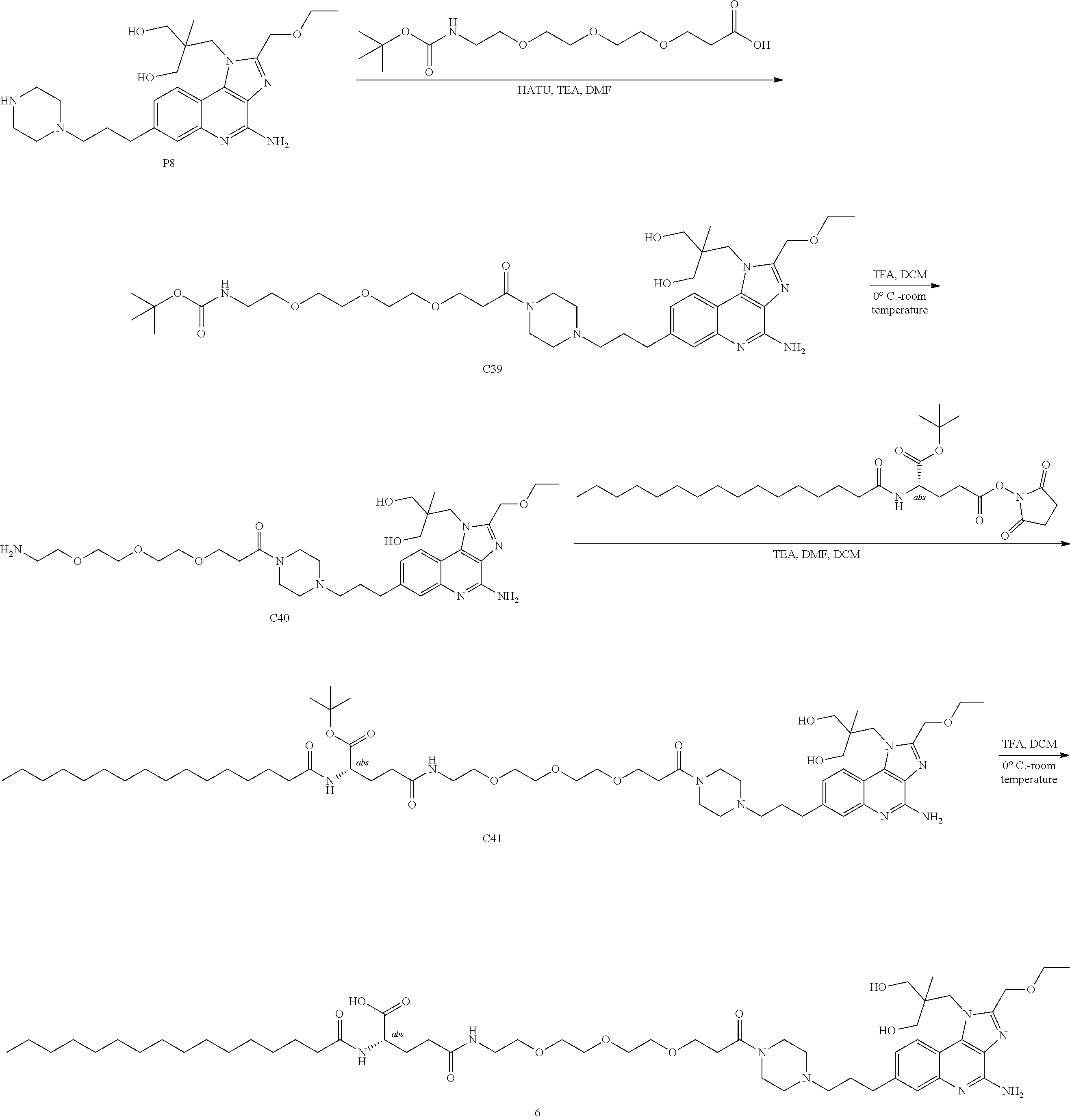

Step 1. Preparation of tert-butyl (2-(2-(2-(3-(4-(3-(4-amino-2-(ethoxymethyl)-1-(3-hydroxy-2-(hydroxymethyl)-2-methylpropyl)-1H-imidazo[4,5-c]quinolin-7-yl)propyl) piperazin-1-yl)-3-oxopropoxy) ethoxy) ethoxy)ethyl) carbamate (C39)

Under N₂ gas, to a stirred solution of P8 (0.200 g, 0.345 mmol) and 2,2-dimethyl-4-oxo-3,8, 11, 14-tetraoxa-5-azaheptadecan-17-oic acid (CAS: 1347750-75-7; 111 mg, 0.345 mmol) in DMF (6 mL) was added HATU (144 mg, 0.379 mmol) then DIPEA (334 mg, 2.59 mmol) successively. The reaction mixture was stirred at room temperature for 16 hours then concentrated in vacuo. The yellow gum was purified by column chromatography (silica gel, (10% NH₄OH in MeOH): DCM, 0-15% of (10% NH₄OH in MeOH)) and lyophilized to provide C39 (0.200 g, 75.0% yield) as a yellow solid. LCMS m/z (M+H)⁺=774.4. ¹H NMR (400 MHz, (CD₃)₂SO) δ 8.70-8.67 (m, 1H), 8.51-8.44 (m, 1H), 7.49-7.42 (m, 1H), 7.20-7.07 (m, 2H), 6.77-6.70 (m, 1H), 5.09-4.95 (m, 2H), 4.87-4.57 (m, 4H), 3.62 (t, 2H), 3.56-3.43 (m, 16H), 3.39-3.35 (m, 4H), 3.01-3.08 (m, 2H), 2.75-2.66 (m, 2H), 2.59-2.53 (m, 2H), 2.43-2.30 (m, 6H), 1.87-1.79 (m, 2H), 1.36 (s, 9H), 1.13 (t, 3H), 0.56 (s, 3H).

Step 2. Preparation of 1-(4-(3-(4-amino-2-(ethoxymethyl)-1-(3-hydroxy-2-(hydroxymethyl)-2-methylpropyl)-1H-imidazo[4,5-c]quinolin-7-yl)propyl) piperazin-1-yl)-3-(2-(2-(2-aminoethoxy)ethoxy)ethoxy)propan-1-one (C40)

At 0-5° C. under N₂ gas, to a stirred solution of C39 (0.200 g, 0.258 mmol) in DCM (3 mL) was added TFA (3 mL) dropwise. The reaction mixture was stirred at room temperature for 1 hour then concentrated in vacuo and lyophilized to provide C40 (0.320 g, >99% yield) as a yellow solid. The solid was used directly in the next step without further purification. LCMS m/z (M+H)⁺=674.4.

Step 3. Preparation of tert-butyl(S)-1-(4-(3-(4-amino-2-(ethoxymethyl)-1-(3-hydroxy-2-(hydroxymethyl)-2-methylpropyl)-1H-imidazo[4,5-c]quinolin-7-yl)propyl) piperazin-1-yl)-1,14-dioxo-17-palmitamido-4,7,10-trioxa-13-azaoctadecan-18-oate trifluoroacetate (C41)

Under N₂ gas, to a stirred solution of C40 (0.320 g, 0.355 mmol) and 1-(tert-butyl) 5-(2,5-dioxopyrrolidin-1-yl) palmitoyl-L-glutamate (CAS: 204521-63-1; 0.127 g, 0.237 mmol) in DCM (4 mL) was added a solution of TEA (0.168 g, 1.66 mmol) in DCM (2 mL) dropwise. The reaction mixture was stirred at room temperature for 10 minutes before DMF (1 mL) was added. The reaction mixture was stirred for an additional 16 hours then concentrated in vacuo. The yellow gum was purified through a pad of silica gel then washed with EtOAc (50 mL). The filtrate was discarded. The pad of silica gel was then washed with (10:1:0.1) DCM: MeOH: NH₄OH (80 mL: 8.0 mL: 0.8 mL). The filtrate was concentrated in vacuo and lyophilized to provide C41 (0.400 g, >99% yield) as a yellow gum. The gum was used directly in the next step without additional purification. LCMS m/z (M)⁺=1097.8.

Step 4. Preparation of(S)-1-(4-(3-(4-amino-2-(ethoxymethyl)-1-(3-hydroxy-2-(hydroxymethyl)-2-methylpropyl)-1H-imidazo[4,5-c]quinolin-7-yl)propyl) piperazin-1-yl)-1, 14-dioxo-17-palmitamido-4.7.10-trioxa-13-azaoctadecan-18-oic acid (6)

At 0-5° C. under N₂ gas, to a stirred solution of C41 (0.400 g, 0.364 mmol) in DCM (6 mL) was added TFA (6 mL) dropwise. The reaction mixture was stirred at room temperature for 1 hour then concentrated in vacuo and lyophilized. The yellow gum was dissolved in (1:1) THF: H₂O (10 mL) then K2CO₃ (0.126 g, 0.911 mmol) was added to pH=9. The suspension was stirred at room temperature for 2 hours then concentrated in vacuo. The yellow gum was purified by reverse phase HPLC (Boston Prime C₁₈ 150 mm×30 mm×5 μm, water (0.05% NH₄OH)/MeCN, 37 to 58% over 10 minutes, 100% MeCN hold time for 2 minutes, flow rate was 25 (mL/min)) then lyophilized to provide 6 (0.100 g, 26.3% yield) as a white solid. LCMS m/z (M+H)⁺=1042.1. ¹H NMR (400 MHz, (CD₃)₂SO) δ 8.47 (d, 1H), 7.90-7.82 (m, 2H), 7.65 (br s, 2H), 7.48-4.67 (m, 1H), 7.18-7.13 (m, 1H), 5.10-4.99 (m, 2H), 4.85-4.74 (m, 1H), 4.69-4.56 (m, 1H), 4.16-4.08 (m, 1H), 3.63-3.58 (m, 4H), 3.49-3.46 (m, 17H), 3.20-3.13 (m, 4H), 2.72 (t, 2H), 2.58-2.53 (m, 4H), 2.40-2.27 (m, 6H), 2.16-2.06 (m, 4H), 2.00-1.90 (m, 1H), 1.86-1.70 (m, 2H), 1.51-1.43 (m, 2H), 1.21 (s, 24H), 1.12 (t, 3H), 0.84 (t, 3H), 0.55 (s, 3H).

The compounds in Table 1 were prepared using general methods or according/analogous to the methods of Schemes 1-6 and Examples 1-6, including modification, as appropriate.

TABLE 1
COMPOUNDS OF EXAMPLES 1-6

| Compound # | |
|---|---|
| and Method | |
| Example | Structure/Compound Name |
| 1 | 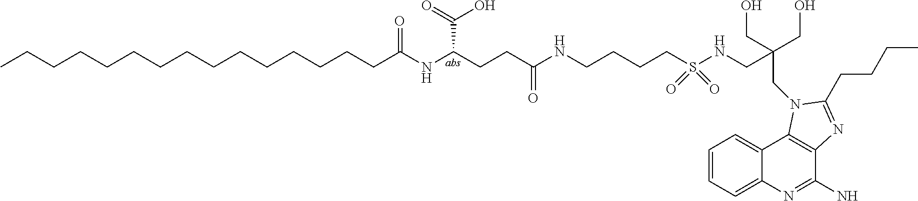 [SMILES: CCCCCCCCCCCCCCCC(=O)N[C@@H](CCC(=O)NCCCCS(=O)(=O)NCC(CO)(CO)Cn1c(CCCC)nc2c(N)nc3ccccc3c21)C(=O)O] N⁵-(4-(N-(3-(4-Amino-2-butyl-1H-imidazo[4,5-c]quinolin-1-yl)-2,2- bis(hydroxymethyl)propyl)sulfamoyl)butyl)-N²-palmitoyl-L-glutamine |
| 2 | 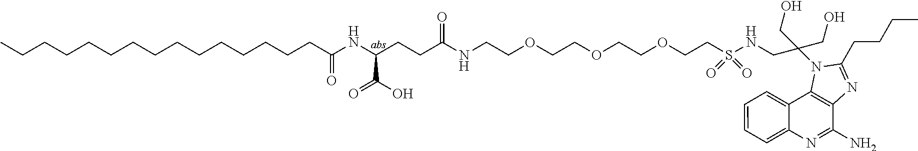 [SMILES: CCCCCCCCCCCCCCCC(=O)N[C@@H](CCC(=O)NCCOCCOCCOCCS(=O)(=O)NCC(CO)(CO)n1c(CCCC)nc2c(N)nc3ccccc3c21)C(=O)O] (S)-1-(N-(3-(4-Amino-2-butyl-1H-imidazo[4,5-c]quinolin-1-yl)-2,2- bis(hydroxymethyl)propyl)sulfamoyl)-13-oxo-16-palmitamido-3,6,9-trioxa-12- azaheptadecan-17-oic acid |
| 3 | 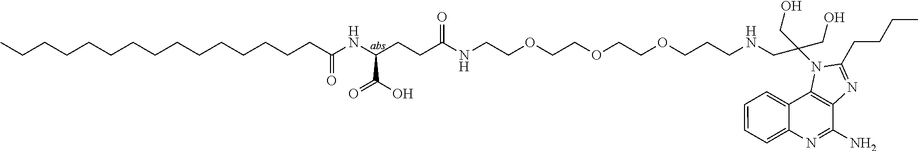 [SMILES: CCCCCCCCCCCCCCCC(=O)N[C@@H](CCC(=O)NCCOCCOCCOCCCNCC(CO)(CO)n1c(CCCC)nc2c(N)nc3ccccc3c21)C(=O)O] (S)-1-(4-Amino-2-butyl-1H-imidazo[4,5-c]quinolin-1-yl)-2,2-bis(hydroxymethyl)-18-oxo-21- palmitamido-8,11,14-trioxa-4,17-diazadocosan-22-oic acid |
| 4 | 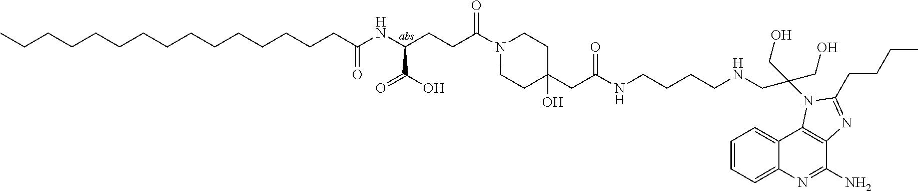 [SMILES: CCCCCCCCCCCCCCCC(=O)N[C@@H](CCC(=O)N1CCC(O)(CC(=O)NCCCCNCC(CO)(CO)n2c(CCCC)nc3c(N)nc4ccccc4c32)CC1)C(=O)O] (S)-5-(4-(2-((4-((3-(4-Amino-2-butyl-1H-imidazo[4,5-c]quinolin-1-yl)-2,2- bis(hydroxymethyl)propyl)amino)butyl)amino)-2-oxoethyl)-4-hydroxypiperidin-1-yl)-5-oxo- 2-palmitamidopentanoic acid |
| 5 | 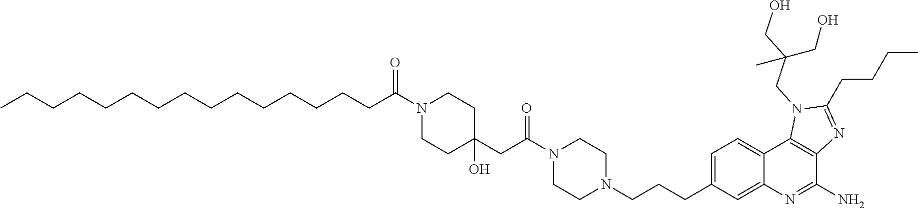 [SMILES: CCCCCCCCCCCCCCCC(=O)N1CCC(O)(CC(=O)N2CCN(CCCc3ccc4c(c3)nc(N)c3nc(CCCC)n(CC(C)(CO)CO)c34)CC2)CC1] 1-(4-(2-(4-(3-(4-Amino-2-butyl-1-(3-hydroxy-2-(hydroxymethyl)-2-methylpropyl)-1H- imidazo[4,5-c]quinolin-7-yl)propyl)piperazin-1-yl)-2-oxoethyl)-4-hydroxypiperidin-1- yl)hexadecan-1-one |
| 6 | 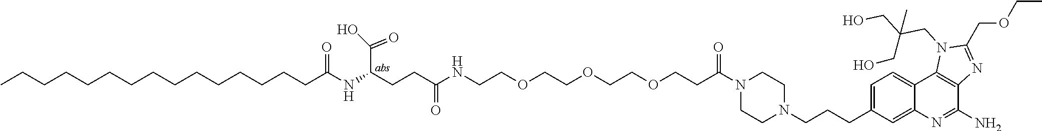 [SMILES: CCCCCCCCCCCCCCCC(=O)N[C@@H](CCC(=O)NCCOCCOCCOCCC(=O)N1CCN(CCCc2ccc3c(c2)nc(N)c2nc(COCC)n(CC(C)(CO)CO)c23)CC1)C(=O)O] (S)-1-(4-(3-(4-Amino-2-(ethoxymethyl)-1-(3-hydroxy-2-(hydroxymethyl)-2-methylpropyl)- 1H-imidazo[4,5-c]quinolin-7-yl)propyl)piperazin-1-yl)-1,14-dioxo-17-palmitamido-4,7,10- trioxa-13-azaoctadecan-18-oic acid |

### Biological Testing

### Example 7

To determine the ability of test compounds to activate the human toll like receptor 7 (hTLR7) or human toll like receptor 8 (hTLR8), cell-based reporter systems were utilized. HEK293 cells stably overexpressing either hTLR7 or hTLR8 along with a reporter gene containing an optimized secreted embryonic alkaline phosphatase gene (SEAP), under the control of the IFN-b minimal promoter fused to five NF-KB and AP-1-binding sites, were obtained from Invivogen (HEK-Blue™ hTLR7, cat #Hkb-htlr7; HEK-Blue™ hTLR8, cat #Hkb-htlr8). Stimulation of hTLR7 or hTLR8 in these cells activates NF-KB and AP-1 and induces the production of SEAP which can be quantified using an alkaline phosphatase detection reagent.

Cells were maintained in DMEM growth media containing heat inactivated (10%), Glutamax (2 mM), Penicillin/Streptomycin, Blasticidin (10 μg/ml), Zeocin (100 μg/ml) and Normocin (100 μg/ml) according to the manufacturer suggestion. On day one of the assay, compounds were prepared using 11-point half-log serial dilutions from a 10 mM DMSO stock solution and 50 nL was spotted into 384-well plates (PerkinElmer, cat #6007480). Positive and negative controls were also spotted within the assay plate and were used to determine percent effect during the analysis process. After resuspension in DMEM assay media containing FBS heat inactivated (10%), Glutamax (2 mM) and Penicillin/Streptomycin, 10,000 cells/20 ul/well were added to previously prepared compound plates. Plates were incubated overnight (16-20 hrs) at 37° C. in a 5% CO₂ environment. Prewetted Microclime lids (Labcyte, LLS-0310) were used to prevent evaporation. On day two of the assay, QUANTI-Blue™ detection reagent was prepared by reconstituting QUANTI-Blue™ powder (InvivoGen, Rep-qb1) with 100 ml of sterile water and allowed to equilibrate to 37° C. for 15 minutes. 20 ul of QUANTI-Blue™ detection reagent was added to each well and plates were incubated at room temperature for 180 min. At the end of the incubation, plates were read on an Envision (Perkin Elmer) plate reader capturing absorbance at 650 nm.

Using Positive (tool compound) and Negative (DMSO) controls, the percent (%) effect was calculated for each sample using the following equation:

$$\% effect = 100 - 100 * ( ( Sample - Positive ) / ( Negative - Positive ) )$$

The % effect at each concentration of compound was calculated utilizing the ABase software suite (IBDS) and was relative to the amount of SEAP produced in the positive and negative control wells contained within each assay plate. The concentrations and % effect values for test compounds were fit using a 4-parameter logistic model in ABase and the concentration of compound that produced 50% response (EC₅₀) was calculated.

TABLE 2

| | hTLR7 | hTLR8 |
|---|---|---|
| | HEK Blue | HEK Blue |
| | EC50 | EC50 |
| Compound/Example Number and Name | (nM) | (nM) |
| 1 | 221 | 316 |
| N⁵-(4-(N-(3-(4-Amino-2-butyl-1H-imidazo[4,5- | | |
| c]quinolin-1-yl)-2,2- | | |
| bis(hydroxymethyl)propyl)sulfamoyl)butyl)-N²- | | |
| palmitoyl-L-glutamine | | |
| 2 | 766 | 2066 |
| (S)-1-(N-(3-(4-Amino-2-butyl-1H-imidazo[4,5- | | |
| c]quinolin-1-yl)-2,2- | | |
| bis(hydroxymethyl)propyl)sulfamoyl)-13-oxo- | | |
| 16-palmitamido-3,6,9-trioxa-12- | | |
| azaheptadecan-17-oic acid | | |
| 3 | 1213 | 2471 |
| (S)-1-(4-Amino-2-butyl-1H-imidazo[4,5- | | |
| c]quinolin-1-yl)-2,2-bis(hydroxymethyl)-18- | | |
| oxo-21-palmitamido-8,11,14-trioxa-4,17- | | |
| diazadocosan-22-oic acid | | |
| 4 | 1390 | 2766 |
| (S)-5-(4-(2-((4-((3-(4-Amino-2-butyl-1H- | | |
| imidazo[4,5-c]quinolin-1-yl)-2,2- | | |
| bis(hydroxymethyl)propyl)amino)butyl)amino)- | | |
| 2-oxoethyl)-4-hydroxypiperidin-1-yl)-5-oxo-2- | | |
| palmitamidopentanoic acid | | |
| 5 | 2938 | 3333 |
| 1-(4-(2-(4-(3-(4-Amino-2-butyl-1-(3-hydroxy-2- | | |
| (hydroxymethyl)-2-methylpropyl)-1H- | | |
| imidazo[4,5-c]quinolin-7-yl)propyl)piperazin-1- | | |
| yl)-2-oxoethyl)-4-hydroxypiperidin-1- | | |
| yl)hexadecan-1-one | | |
| 6 | 1026 | 3449 |
| (S)-1-(4-(3-(4-Amino-2-(ethoxymethyl)-1-(3- | | |
| hydroxy-2-(hydroxymethyl)-2-methylpropyl)- | | |
| 1H-imidazo[4,5-c]quinolin-7- | | |
| yl)propyl)piperazin-1-yl)-1,14-dioxo-17- | | |
| palmitamido-4,7,10-trioxa-13-azaoctadecan- | | |
| 18-oic acid | | |

### Liposomal Formulation

### Example 8

A liposome containing a lipidated TLR 7/8 modulating compound was prepared in order to create a liposomal adjuvant. Specifically, N⁵-(4-(N-(3-(4-Amino-2-butyl-1H-imidazo[4,5-c]quinolin-1-yl)-2,2-bis(hydroxymethyl)propyl)sulfamoyl)butyl)-N²-palmitoyl-L-glutamine (Compound 1 shown in Table 1 above) was incorporated into a liposomal formulation. The manufacturing process included the formulating of aqueous and organic phases, mixing in a microfluidic device, tangential flow filtration (TFF), bioburden reduction filtration (BBR), sterile filtration, and aseptic compounding with QS-21.

### Solution Preparation

The first manufacturing step involved formulating the aqueous and organic phases. The aqueous formulation buffer contained 10 mM phosphate and 150 mM NaCl, the buffer had a pH of 6.2. The aqueous formulation buffer was filtered through a 0.2 μm filter prior to use.

The organic phase composition contained the following components: 0.0688 mg/ml of Compound 1, 28 mg/ml of DMPC, 3.2 mg/ml of DMPG, and 22 mg/ml of cholesterol. The components were then dissolved in ethanol at ˜65° C. to make a clear solution.

### Microfluidic T-Mixing

The liposome was formed by mixing the aqueous formulation buffer with the organic phase through a microfluidic T-mixer (IDEX® U-429). Specifically, 600 mL of aqueous formulation buffer at ambient room temperature was mixed with 200 mL of organic phase at 45-65° C. The volumetric flowrate of aqueous formulation buffer and organic phase were set at 180 mL/min and 60 mL/min, respectively. The first ˜50 mL of effluent was discarded as the ‘leading edge’ to ensure that the liposomes were not collected prior to the system reaching steady state. The resulting liposome preparation was a white homogeneous suspension.

### Tangential Flow Filtration (TFF)

After microfluidic T-mixing, the liposome preparation was processed via TFF to remove ethanol and to concentrate the liposome to approximately 0.1-0.17 g/L of Compound 1 (the target was 0.15 mg/mL). 1 piece of membrane with a molecular weight cutoff (MWCO) at 100 kDa was used for TFF. The membrane is used at <570 g of total lipids per m² of membrane area. The feed pressure was controlled to a target of <28 psi and the transmembrane pressure was maintained within a range of 6-12 psi. The nominal process endpoint was based on achieving a target concentration of 0.15 mg/mL of Compound 1.

### Bioburden Reduction Filtration (BBR) Of Liposome

Following TFF, the liposome preparation underwent filtration to reduce the level of bioburden and to improve subsequent filterability. Specifically, the liposome preparation was collected into a 30 mL sterile luer lock syringe. A Sartoguard™ 0.2 μm nominal capsule filter was connected to the syringe. The liposome preparation was filtered through the membrane into a sterile bottle. The process was repeated until all of the liposome preparation was filtered.

### Sterile Filtration

Next, the liposome preparation was sterilized. Specifically, the liposome preparation was collected into a 30 mL sterile luer lock syringe. A 0.45/0.2 μm capsule filter (Sartopore® 2) was connected to the syringe. The liposome preparation was filtered through the membrane into a sterile bottle. The liposome preparation was stored at 2-8° C.

### Addition of QS-21

The sterilized liposome preparation was aseptically compounded with QS-21 to form the QS-21 containing liposomes. Specifically, the liposome preparation was dispensed into 4 appropriate compounding vessels. The liposome preparation was gently mixed at a slight vortex. 4 stock solutions of QS-21 were prepared with 0.2-0.8 mg/mL QS-21 in buffer with 10 mM phosphate and 150 mM NaCl, pH 6.2. Compounding was performed to achieve concentration targets of 0.0688 mg/mL of Compound 1 and 0.1-0.4 mg/ml of QS-21, respectively. The stock solutions were respectively added to the liposome preparation using a pipette at 1:1 volume ratio with subsequent gentle mixing for 2 hours at room temperature. The QS-21 containing liposome preparations were white homogeneous suspensions after compounding. The final QS-21 containing liposome adjuvant product was stored at 2-8° C.

### Size and PDI Measurement

As shown in Table 3 below, the size (nm) and polydispersity index (PDI) of the liposomes within liposomal preparations with and without QS-21 was measured by Dynamic light scattering (DLS) over a time frame of 3 months. The size and PDI were slightly increased at 3 months when QS-21 was added to the liposomes. The liposomes without QS-21 had a more stable size and PDI over the course of 3 months.

TABLE 3
Size and PDI of Liposomes With and Without QS-21

| | Compound 1 | QS-21 | DMPC | DMPG | Cholesterol | Size (nm) | | | PDI | | |
|---|---|---|---|---|---|---|---|---|---|---|---|
| # | mg/mL | mg/mL | mg/mL | mg/mL | (mg/mL) | T0 | 1 M | 3 M | T0 | 1 M | 3 M |
| 1 | 0.0688 | 0 | 28 | 3.2 | 21.6 | 108 | 108 | 108 | 0.156 | 0.14 | 0.132 |
| 2 | 0.0688 | 0.1 | 28 | 3.2 | 21.6 | 112 | 115 | 116 | 0.129 | 0.155 | 0.168 |
| 3 | 0.0688 | 0.2 | 28 | 3.2 | 21.6 | 116 | 122 | 126 | 0.166 | 0.191 | 0.217 |
| 4 | 0.0688 | 0.3 | 28 | 3.2 | 21.6 | 123 | 133 | 143 | 0.222 | 0.253 | 0.289 |
| 5 | 0.0688 | 0.4 | 28 | 3.2 | 21.6 | 132 | 153 | 156 | 0.295 | 0.337 | 0.41 |

### Hemolytic Assay

The cytotoxicity of liposomes with various concentrations of QS-21 was tested in a hemolytic assay. The formulations tested corresponded to samples 2-5 as shown in Table 3 above. No hemolytic activity was observed for the formulations tested, as shown in Table 4, below.

TABLE 4
Hemolytic Activity of Liposomes with QS-21

| | Compound 1 | QS-21 concentration | Hemolytic |
|---|---|---|---|
| # | (mg/mL) | (mg/mL) | activity |
| 2 | 0.0688 | 0.1 | No |
| 3 | 0.0688 | 0.2 | No |
| 4 | 0.0688 | 0.3 | No |
| 5 | 0.0688 | 0.4 | No |

Accordingly, this example demonstrates the process for formulating a liposome that contains a lipidated TLR 7/8 modulating molecule, QS-21, DMPC, DMPG, and cholesterol. Furthermore, these liposomes maintain an average size below 200 nm over the course of 3 months. Additionally, the liposomes did not demonstrate cytotoxicity as measured by the lack of hemolytic activity.

### Immunogenicity Evaluation of Liposomal Formulation in a Mouse Model

### Example 9

A murine in vivo study was completed to evaluate the ability of a liposomal formulation containing a TLR 7/8 modulating molecule (Compound 1, Table 1) to adjuvant the immune response induced by a pneumococcal serotype 3 (PnC3) antigen conjugated to CRM197. Previous studies have demonstrated that the inclusion of Toll-like receptor (TLR) agonists in vaccine formulations enhances antigen presentation, promotes dendritic cell maturation, and stimulates the activation of B and T lymphocytes, thereby improving vaccine immunogenicity.

The study design is provided in Table 5, below. All groups in the study received the PnC3 antigen at a dose of 0.1 μg.

As shown in Table 5, below, groups 3-8 were administered liposomal formulations containing various amounts of Compound 1 (3.44 μg, 0.86 μg, or 0.17 μg per dose), as well as DMPC, DMPG, and cholesterol (concentrations provided in Table 6). The liposomal formulations administered to groups 6-8 further contained QS-21 at a concentration of 5 μg per dose.

As shown in Table 5, below, Groups 1 and 2 were administered comparator adjuvants. An aluminum phosphate adjuvant was administered to Group 1. A liposomal adjuvant comprising DMPC, DMPG, cholesterol, 3D-PHAD™ (i.e., monophosphoryl 3-Deacyl Lipid A (synthetic) available from Avanti® polar lipids), and QS-21 was administered to Group 2. The detailed composition of the liposomal adjuvants is provided in Table 6, below.

TABLE 5
Study Design

| | | | Compound 1 | Vaccine |
|---|---|---|---|---|
| | | Adjuvant | Concentration | Time |
| Group | Adjuvant | dose | (nmol/50 μL) | Points |
| 1 | AlPO₄ | 100 μg | — | 0, 3 |
| 2 | Liposomal Adjuvant with 3D- | 20 μg | — | 0, 3 |
| | PHAD ™ and QS-21 | 3D-PHAD ™ | | |
| | | and10 μg | | |
| | | QS-21 | | |
| 3 | Liposomal Adjuvant with | 3.44 μg | 4 | 0, 3 |
| | Compound 1 | Compound 1 | | |
| 4 | Liposomal Adjuvant with | 0.86 μg | 1 | 0, 3 |
| | Compound 1 | Compound 1 | | |
| 5 | Liposomal Adjuvant with | 0.17 μg | 0.2 | 0, 3 |
| | Compound 1 | Compound 1 | | |
| 6 | Liposomal Adjuvant with | 3.44 μg | 4 | 0, 3 |
| | Compound 1 and | Compound 1 | | |
| | QS-21 | 5 μg | | |
| | | QS-21 | | |
| 7 | Liposomal Adjuvant with | 1.72 μg | 2 | 0, 3 |
| | Compound 1 and | Compound 1 | | |
| | QS-21 | 5 μg | | |
| | | QS-21 | | |
| 8 | Liposomal Adjuvant with | 0.86 μg | 1 | 0, 3 |
| | Compound 1 and | Compound 1 | | |
| | QS-21 | 5 μg | | |
| | | QS-21 | | |

TABLE 6
Liposomal Adjuvant Composition

| | | Compound 1 | QS-21 | MPLA | DMPC(mg/ | DMPG | Cholesterol |
|---|---|---|---|---|---|---|---|
| # | Adjuvant | (mg/mL) | (mg/mL) | (mg/mL) | mL) | (mg/mL) | (mg/mL) |
| 2 | Liposomal | — | 0.2 | 0.4 | 14 | 1.6 | 10.8 |
| | Adjuvant with | | | | | | |
| | 3D-PHAD ™ | | | | | | |
| | and QS-21 | | | | | | |
| 3 | Liposomal | 0.0688 | — | — | 56 | 6.4 | 43.2 |
| | Adjuvant with | | | | | | |
| | Compound 1 | | | | | | |
| 4 | Liposomal | 0.0172 | — | — | 14 | 1.6 | 10.8 |
| | Adjuvant with | | | | | | |
| | Compound 1 | | | | | | |
| 5 | Liposomal | 0.0034 | — | — | 2.8 | 0.32 | 2.16 |
| | Adjuvant with | | | | | | |
| | Compound 1 | | | | | | |
| 6 | Liposomal | 0.0688 | 0.1 | — | 56 | 6.4 | 43.2 |
| | Adjuvant with | | | | | | |
| | Compound 1 | | | | | | |
| | and | | | | | | |
| | QS-21 | | | | | | |
| 7 | Liposomal | 0.0344 | 0.1 | — | 28 | 3.2 | 21.6 |
| | Adjuvant with | | | | | | |
| | Compound 1 | | | | | | |
| | and | | | | | | |
| | QS-21 | | | | | | |
| 8 | Liposomal | 0.0172 | 0.1 | — | 14 | 1.6 | 10.8 |
| | Adjuvant with | | | | | | |
| | Compound 1 | | | | | | |
| | and | | | | | | |
| | QS-21 | | | | | | |

In brief, the materials and methods for the mouse studies were as follows: Swiss Webster mice (10/group, 6-8 weeks old, TACONIC BIOSCIENCES®) were immunized intramuscularly (IM) at 0 and 3 weeks. Mice received 50 ul of 0.1 μg PnC3 antigen intramuscularly in combination with one of the eight adjuvant formulations provided in Table 5. At 5 weeks post prime vaccination all mice were terminally bled while under anesthesia and the collected sera was used to determine opsonophagocytic assay (OPA) titers.

The OPA geometric mean titers are provided in Table 7, below. In mice vaccinated with the liposomal adjuvant containing Compound 1 but without QS-21, the formulation with the higher Compound 1 concentration (3.44 μg) induced a higher average OPA titer. Conversely, in mice vaccinated with the liposomal adjuvant containing Compound 1 and QS-21, the formulation with the lowest Compound 1 dose (0.86 μg) had the highest average geomean OPA titer.

TABLE 7
OPA Geomean Titers ± 95% Confidence Intervals

| | | Adjuvant | | Lower | Upper |
|---|---|---|---|---|---|
| Group | Adjuvant | dose | GMT | 95% CI | 95% CI |
| 1 | AlPO₄ | 100 μg | 1067 | 215 | 5287 |
| 2 | Liposomal Adjuvant with 3D- | 20 μg | 4473 | 1327 | 15078 |
| | PHAD ™ and QS-21 | 3D- | | | |
| | | PHAD ™ | | | |
| | | and10 μg | | | |
| | | QS-21 | | | |
| 3 | Liposomal Adjuvant with | 3.44 μg | 5227 | 2983 | 9158 |
| | Compound 1 | Compound | | | |
| | | 1 | | | |
| 4 | Liposomal Adjuvant with | 0.86 μg | 4309 | 646 | 28732 |
| | Compound 1 | Compound | | | |
| | | 1 | | | |
| 5 | Liposomal Adjuvant with | 0.17 μg | 665 | 345 | 1283 |
| | Compound 1 | Compound | | | |
| | | 1 | | | |
| 6 | Liposomal Adjuvant with | 3.44 μg | 2096 | 516 | 8512 |
| | Compound 1 and | Compound | | | |
| | QS-21 | 1 | | | |
| | | 5 μg | | | |
| | | QS-21 | | | |
| 7 | Liposomal Adjuvant with | 1.72 μg | 2753 | 282 | 26912 |
| | Compound 1 and | Compound | | | |
| | QS-21 | 1 | | | |
| | | 5 μg | | | |
| | | QS-21 | | | |
| 8 | Liposomal Adjuvant with | 0.86 μg | 7756 | 3349 | 17965 |
| | Compound 1 and | Compound | | | |
| | QS-21 | 1 | | | |
| | | 5 μg | | | |
| | | QS-21 | | | |

### Example 10

A murine in vivo study was completed to evaluate the ability of a liposomal formulation containing a TLR 7/8 modulating molecule (Compound 6, Table 1) to adjuvant the immune response induced by a pneumococcal serotype 3 (PnC3) antigen conjugated to CRM197. Previous studies have demonstrated that the inclusion of Toll-like receptor (TLR) agonists in vaccine formulations enhances antigen presentation, promotes dendritic cell maturation, and stimulates the activation of B and T lymphocytes, thereby improving vaccine immunogenicity.

The study design is provided in Table 8, below. All groups in the study received the PnC3 antigen at a dose of 0.1 μg.

As shown in Table 8, below, groups 3-7 were administered liposomal formulations containing various amounts of Compound 6 (4.164 μg, 2.082 μg, or 1.041 μg per dose), as well as DMPC, DMPG, and cholesterol (concentrations provided in Table 9). The liposomal formulations administered to groups 5-7 further contained QS-21 at a concentration of 5 μg per dose.

As shown in Table 8, below, Groups 1 and 2 were administered comparator adjuvants. An aluminum phosphate adjuvant was administered to Group 1. A liposomal adjuvant comprising DMPC, DMPG, cholesterol, 3D-PHAD™ (i.e., monophosphoryl 3-Deacyl Lipid A (synthetic) available from Avanti® polar lipids), and QS-21 was administered to Group 2. The detailed composition of the liposomal adjuvants is provided in Table 9, below.

TABLE 8
Study Design

| | | | Compound 1 | Vaccine |
|---|---|---|---|---|
| | | Adjuvant | Concentration | Time |
| Group | Adjuvant | dose | (nmol/50 μL) | Points |
| 1 | AlPO₄ | 100 μg | — | 0, 3 |
| 2 | Liposomal Adjuvant with 3D- | 10 μg | — | 0, 3 |
| | PHAD ™ and QS-21 | 3D-PHAD ™ | | |
| | | and 5 μg | | |
| | | QS-21 | | |
| 3 | Liposomal Adjuvant with | 4.164 μg | 4 | 0, 3 |
| | Compound 6 | Compound | | |
| | | 6 | | |
| 4 | Liposomal Adjuvant with | 1.041 μg | 1 | 0, 3 |
| | Compound 6 | Compound | | |
| | | 6 | | |
| 5 | Liposomal Adjuvant with | 4.164 μg | 4 | 0, 3 |
| | Compound 6 and QS-21 | Compound | | |
| | | 6 | | |
| | | 5 μg | | |
| | | QS-21 | | |
| 6 | Liposomal Adjuvant with | 2.082 μg | 2 | 0, 3 |
| | Compound 6 and | Compound | | |
| | QS-21 | 6 | | |
| | | 5 μg | | |
| | | QS-21 | | |
| 7 | Liposomal Adjuvant with | 1.041 μg | 1 | 0, 3 |
| | Compound 6 and | Compound | | |
| | QS-21 | 6 | | |
| | | 5 μg | | |
| | | QS-21 | | |

TABLE 9
Liposomal Adjuvant Composition

| | | TLR7/8 | | | |
|---|---|---|---|---|---|
| | | Conc. | DMPC | DMPG | Cholesterol |
| | | (nmol/ | (nmol/ | (nmol/ | (nmol/ |
| Group | Adjuvant | 50 μL) | 50 μl) | 50 μl) | 50 μl) |
| 1 | AlPO₄ | — | — | — | — |
| 2 | Liposomal | — | 516 | 58 | 698 |
| | Adjuvant with | | | | |
| | 3D-PHAD ™ | | | | |
| | and QS-21 | | | | |
| 3 | Liposomal | 4 | 2065 | 232 | 2793 |
| | Adjuvant with | | | | |
| | Compound 6 | | | | |
| 4 | Liposomal | 1 | 516 | 58 | 698 |
| | Adjuvant with | | | | |
| | Compound 6 | | | | |
| 5 | Liposomal | 4 | 2065 | 232 | 2793 |
| | Adjuvant with | | | | |
| | Compound 6 | | | | |
| | and QS-21 | | | | |
| 6 | Liposomal | 2 | 1032 | 116 | 1396 |
| | Adjuvant with | | | | |
| | Compound 6 | | | | |
| | and | | | | |
| | QS-21 | | | | |
| 7 | Liposomal | 1 | 516 | 58 | 698 |
| | Adjuvant with | | | | |
| | Compound 6 | | | | |
| | and | | | | |
| | QS-21 | | | | |

In brief, the materials and methods for the mouse studies were as follows: Swiss Webster mice (10/group, 6-8 weeks old, TACONIC BIOSCIENCES®) were immunized intramuscularly (IM) at 0 and 3 weeks. Mice received 50 ul of 0.1 μg PnC3 antigen intramuscularly in combination with one of the seven adjuvant formulations provided in Table 8. At 5 weeks post prime vaccination all mice were terminally bled while under anesthesia and the collected sera was used to determine opsonophagocytic assay (OPA) titers.

The OPA geometric mean titers are provided in Table 10, below. The OPA titers elicited by mice vaccinated with Compound 6 at 2 nmol (p=0.0135) and 4 nmol (p=0.0027), and adjuvanted with QS-21, were significantly higher than the AIPO₄ control. In mice vaccinated with Compound 6 with or without QS-21, the higher TLR7/8 concentration (4.164 μg) induced a dose specific response. No adverse events were observed throughout the duration of the study, nor any out-of-range weight loss.

TABLE 10
OPA Geomean Titers ± 95% Confidence Intervals

| | | Adjuvant | | Lower | Upper |
|---|---|---|---|---|---|
| Group | Adjuvant | dose | GMT | 95% CI | 95% CI |
| 1 | AlPO₄ | 100 μg | 197 | 42 | 920 |
| 2 | Liposomal Adjuvant | 10 μg 3D- | 2594 | 1805 | 3729 |
| | with 3D-PHAD ™ | PHAD/5 | | | |
| | and QS-21 | μg QS-21 | | | |
| 3 | Liposomal Adjuvant | 4.164 μg | 1351 | 777 | 2350 |
| | with Compound 6 | Compound | | | |
| | | 6 | | | |
| 4 | Liposomal Adjuvant | 1.041 μg | 257 | 61 | 1078 |
| | with Compound 6 | Compound | | | |
| | | 6 | | | |
| 5 | Liposomal Adjuvant | 4.164 μg | 3611 | 1847 | 7059 |
| | with Compound 6 | Compound | | | |
| | and QS-21 | 6 + 5 μg | | | |
| | | QS-21 | | | |
| 6 | Liposomal Adjuvant | 2.082 μg | 2843 | 1441 | 5611 |
| | with Compound 6 | Compound | | | |
| | and QS-21 | 6 + 5 μg | | | |
| | | QS-21 | | | |
| 7 | Liposomal Adjuvant | 1.041 μg | 1772 | 973 | 3230 |
| | with Compound 6 | Compound | | | |
| | and QS-21 | 6 + 5 μg | | | |
| | | QS-21 | | | |

1. A compound of Formula (I): 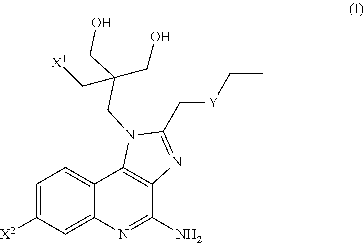
or a pharmaceutically acceptable salt thereof, wherein:
Y is —O— or —CH₂—; and
one of X¹ and X² is H, and the other of X¹ and X² has a formula selected from the group consisting of formula (a), formula (b), and formula (c): 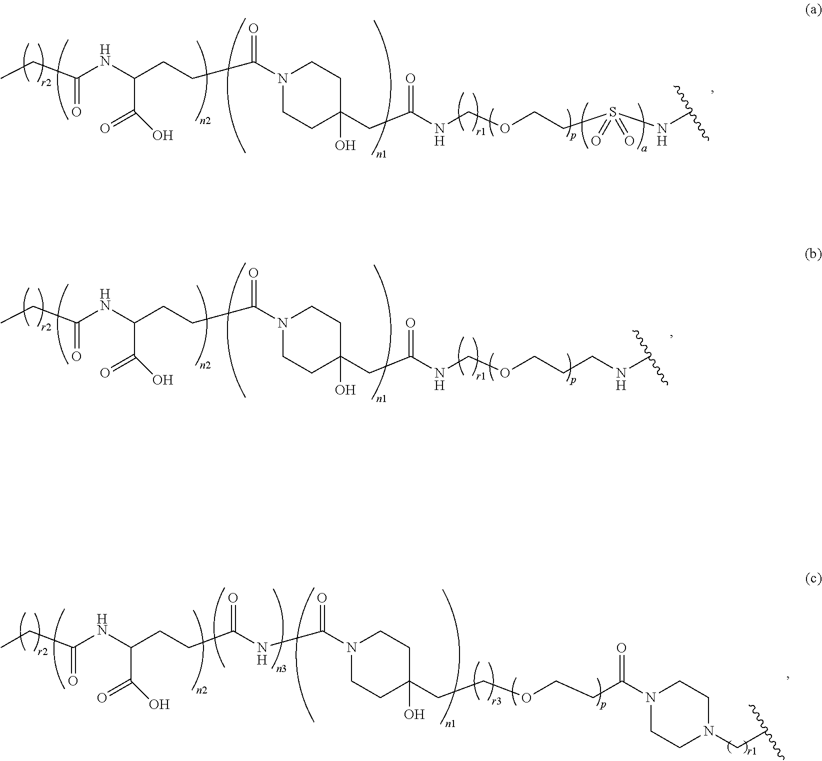
wherein:
a is 0 or 1;
r1 is an integer from 2 to 6;
r2 is an integer from 10 to 20;
r3 is an integer from 0 to 6;
n1 is 0 or 1 and n2 is 0 or 1, wherein at least one of n1 and n2 is 1;
n3 is 0 or 1; and
p is an integer from 0 to 6.

2. The compound of claim 1, wherein X¹ has formula a.

3. The compound of claim 2, wherein a is 1, n1 is 0, n2 is 1, r1 is 4, and p is 0.

4. (canceled)

5. (canceled)

6. The compound of claim 2, wherein a is 1, n1 is 0, n2 is 1, r1 is 2, and p is 3.

7. (canceled)

8. The compound of claim 1, wherein X¹ has formula b.

9. The compound of claim 8, wherein n1 is 0, n2 is 1, r1 is 2, and p is 3.

10. (canceled)

11. The compound of claim 8, wherein n1 is 1, n2 is 1, r1 is 3, and p is 0.

12. (canceled)

13. The compound of claim 1, wherein X² has formula c.

14. The compound of claim 13, wherein n1 is 1, n2 is 0, n3 is 0, r1 is 3, r3 is 0, and p is 0.

15. (canceled)

16. The compound of claim 13, wherein n1 is 0, n2 is 1, n3 is 1, r1 is 3, 13 is 2, and p is 3.

17. (canceled)

18. The compound of claim 1, or a pharmaceutically acceptable salt thereof, wherein r2 is 13, 14, or 15.

19. (canceled)

20. A compound, which is N⁵-(4-(N-(3-(4-Amino-2-butyl-1H-imidazo[4,5-c]quinolin-1-yl)-2,2-bis(hydroxymethyl)propyl)sulfamoyl)butyl)-N²-palmitoyl-L-glutamine, or a pharmaceutically acceptable salt thereof; or
a compound, which is(S)-1-(N-(3-(4-Amino-2-butyl-1H-imidazo[4,5-c]quinolin-1-yl)-2,2-bis(hydroxymethyl)propyl)sulfamoyl)-13-oxo-16-palmitamido-3,6,9-trioxa-12-azaheptadecan-17-oic acid, or a pharmaceutically acceptable salt thereof; or
a compound, which is(S)-1-(4-Amino-2-butyl-1H-imidazo[4,5-c]quinolin-1-yl)-2.2-bis(hydroxymethyl)-18-oxo-21-palmitamido-8,11,14-trioxa-4,17-diazadocosan-22-oic acid, or a pharmaceutically acceptable salt thereof; or
a compound, which is(S)-5-(4-(2-((4-((3-(4-Amino-2-butyl-1H-imidazo[4,5-c]quinolin-1-yl)-2.2-bis(hydroxymethyl)propyl)amino)butyl) amino)-2-oxoethyl)-4-hydroxypiperidin-1-yl)-5-oxo-2-palmitamidopentanoic acid, or a pharmaceutically acceptable salt thereof; or
a compound, which is 1-(4-(2-(4-(3-(4-Amino-2-butyl-1-(3-hydroxy-2-(hydroxymethyl)-2-methylpropyl)-1H-imidazo[4,5-c]quinolin-7-yl)propyl) piperazin-1-yl)-2-oxoethyl)-4-hydroxypiperidin-1-yl) hexadecan-1-one, or a pharmaceutically acceptable salt thereof; or
a compound, which is(S)-1-(4-(3-(4-Amino-2-(ethoxymethyl)-1-(3-hydroxy-2-(hydroxymethyl)-2-methylpropyl)-1H-imidazo[4,5-c]quinolin-7-yl)propyl) piperazin-1-yl)-1,14-dioxo-17-palmitamido-4,7,10-trioxa-13-azaoctadecan-18-oic acid, or a pharmaceutically acceptable salt thereof.

21-25. (canceled)

26. A pharmaceutical composition comprising the compound according to claim 1, or a pharmaceutically acceptable salt thereof, and at least one pharmaceutically acceptable excipient.

27. A crystalline form of the compound according to claim 1 or a pharmaceutically acceptable salt thereof.

28. A method of inducing an immune response to an antigen of interest in a subject, comprising administering to the subject the pharmaceutical composition of claim 26, wherein the composition further comprises the antigen of interest; or
a method for immunizing a subject against a disease or disorder caused by or associated with an antigen of interest, comprising administering to the subject the pharmaceutical composition of claim 26, wherein the composition further comprises the antigen of interest; or
a method for preventing a disease or disorder caused by or associated with an antigen of interest in a subject, comprising administering to the subject the pharmaceutical composition of claim 26, wherein the composition further comprises the antigen of interest; or
a method for treating a disease or disorder caused by or associated with an antigen of interest in a subject, comprising administering to the subject the pharmaceutical composition of claim 26, wherein the composition further comprises the antigen of interest; or
a method for increasing an immune response to an antigen of interest in a subject, comprising administering to the subject the pharmaceutical composition of claim 26, wherein the composition further comprises the antigen of interest.

29-52. (canceled)

53. A liposomal formulation, comprising a plurality of liposomes, wherein the liposomes comprise a compound of Formula (I): 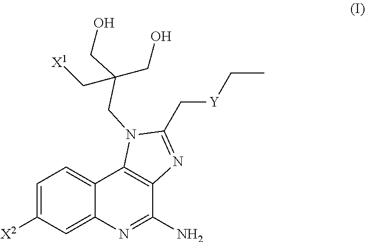
or a pharmaceutically acceptable salt thereof, wherein:
Y is —O— or —CH₂—; and
one of X¹ and X² is H, and the other of X¹ and X² has a formula selected from the group consisting of formula (a), formula (b), and formula (c): 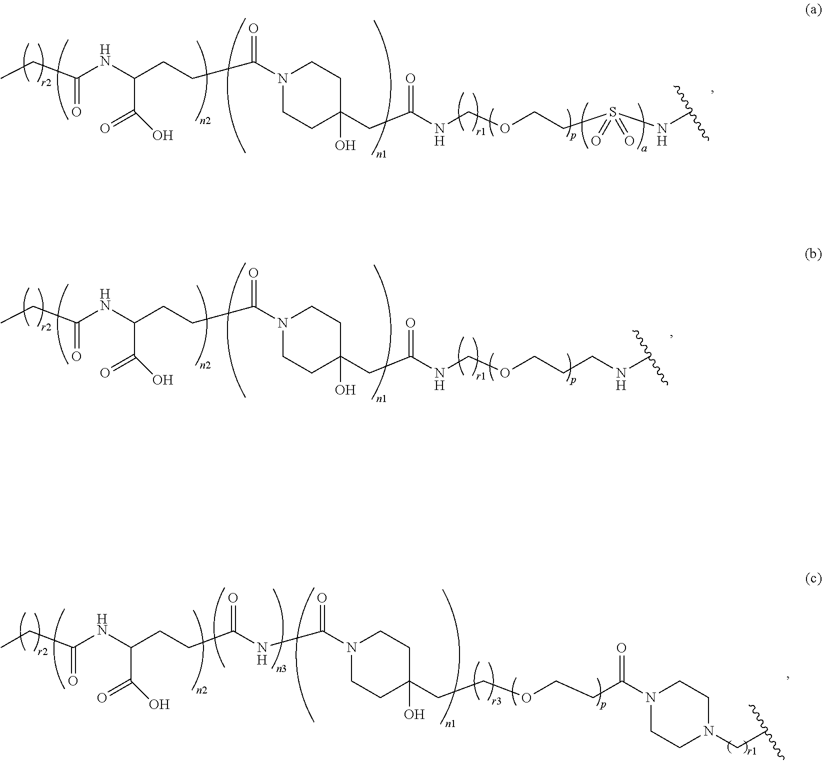
wherein:
a is 0 or 1;
r1 is an integer from 2 to 6;
r2 is an integer from 10 to 20;
r3 is an integer from 0 to 6;
n1 is 0 or 1 and n2 is 0 or 1, wherein at least one of n1 and n2 is 1;
n3 is 0 or 1; and
p is an integer from 0 to 6.

54-77. (canceled)

78. The liposomal formulation of claim 53, wherein the liposomes further comprise:
a) a phospholipid; and
b) cholesterol.

79-109. (canceled)

110. An immunogenic composition comprising the liposomal formulation of claim 78 and an immunogen.

111-115. (canceled)

116. A method of inducing an immune response in a subject against the immunogen, comprising administering to the subject the immunogenic composition of claim 110; or
a method for immunizing a subject against a disease or disorder caused by or associated with the immunogen, comprising administering to the subject the immunogenic composition of claim 110; or
a method for preventing a disease or disorder caused by or associated with the immunogen in a subject, comprising administering to the subject the immunogenic composition of claim 110; or
a method for treating a disease or disorder caused by or associated with the immunogen in a subject, comprising administering to the subject the immunogenic composition of claim 110; or
a method for increasing an immune response to the immunogen in a subject, comprising administering to the subject the immunogenic composition of claim 110.

117-136. (canceled)

137. A method for producing a liposomal formulation, comprising a plurality of liposomes, wherein the liposomes comprise (a) the compound of claim 1, (b) a saponin, (c) a phospholipid or phospholipids selected from the group consisting of dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), and distearyl phosphatidylglycerol (DSPG), and (d) cholesterol, wherein the method comprises the following steps:
(i) dissolving the phospholipid(s), cholesterol, and the compound of claim 1 in an organic solvent to form an organic phase;
(ii) mixing the organic phase of step (i) into an aqueous phase, wherein the aqueous phase comprises a buffer or water, in a microfluidic device to form an intermediate liposome;
(iii) removing the organic solvent of the intermediate liposome of step (ii);
(iv) concentrating the intermediate liposome of step (iii);
(v) filtering the intermediate liposome of step (iv); and
(vi) aseptically mixing the intermediate liposome of step (v) and the saponin.

138-143. (canceled)
